(19) United States
(12) Patent Application Publication
(10) Pub. No.: US 2026/0176593 A1
(43) Pub. Date: Jun. 25, 2026

(54) Methods and Compositions for Preparing Recombinant Adeno Associated Viruses and Uses Thereof

(71) Applicant: National Resilience, Inc., San Diego, CA (US)

(72) Inventors: Eduardo Gabriel Bermudez AGUILAR, San Diego, CA (US); Brandon Samir RAZOOKY, San Diego, CA (US); Michael Paul GAPINSKE, San Diego (CA); Bryan ST. GERMAIN, San Diego, CA (US); John KERWIN, San Diego, CA (US)

(21) Appl. No.: 19/126,974

(22) PCT Filed: Nov. 3, 2023
(86) PCT No.: PCT/US2023/078742

§ 371 Date: May 2, 2025

Related U.S. Application Data
(60) Provisional application No. 63/382,334, filed on Nov. 4, 2022.

(51) Int. Cl. C12N 7/00

(52) U.S. Cl. CPC C12N 7/00; C12N 2750/14143

(57) ABSTRACT

Disclosed herein are nucleic acid molecules and compositions of preparing recombinant adeno-associated viruses (rAAV) and uses thereof. Specifically, two-plasmid systems are provided for efficient rAAV production.

### CROSS REFERENCE

This application claims priority to U.S. Provisional Application No. 63/382,334, filed on Nov. 4, 2022, which is incorporated herein by reference in its entirety.

### BACKGROUND

Adeno associated virus (AAV) can be a single-stranded DNA parvovirus which can integrate into a host genome during the latent phase of infectivity. AAV can be considered a dependovirus since in some cases it can require helper functions from either adenovirus or herpes virus in order to replicate productively. In the absence of either of these helper functions, AAV can infect cells, uncoat in the nucleus, and integrate its genome into the host cell chromosome, but in some cases cannot replicate or produce new viral particles.

The genome of AAV can comprise two terminal repeats, which can serve as origins of DNA replication, and two functional regions. One functional region can provide rep genes which can regulate viral DNA replication and viral gene expression, whereas the second functional region can provide cap genes which can encode the structural capsid proteins VP1, VP2 and VP3. The proteins encoded by both the rep and cap genes can function in trans during productive AAV replication.

AAVs can be ideal for gene therapy due to their low immunogenicity, restricted generation of neutralizing antibodies, and replication defectiveness. AAV production can have cytopathogenic effects, which can occur after co-infection with a helper virusHelper viruses can be difficult to remove and can induce undesired effects such as inflammation in the host.

A need exists for an improved rAAV production platform for efficient transfection of cells and high-yield production of rAAV particles with less impurities.

### SUMMARY

Disclosed herein are isolated nucleic acid molecules comprising (a) a sequence encoding an AAV capsid, wherein isolated nucleic acid molecule does not comprise a sequence encoding AAV rep; and (b) an exogenous gene flanked by inverted terminal repeat (ITR) sequences.

In some embodiments, the AAV capsid comprises a capsid from AAV1, AAV2, AAV2 variant, AAV3A, AAV3B, AAV4, AAV5, AAV6, AAV7, AAV8, AAV9, AAV9 variant, AAV10, AAV11, AAV12, AAV13, AAV-Rh10, AAV-Rh74, AAV-2i8, AAV-DJ, LK03, NP59, or KP1. In some embodiments, the exogeneous gene has a size from about 2 kilobases to about 7 kilobases. In some embodiments, the isolated nucleic acid molecule further comprises a Ef1α promoter 5′ of the sequence coding the AAV capsid. In some embodiments, the isolated nucleic acid molecule further comprises a p40 promoter 5′ of the sequence coding the AAV capsid.

Disclosed herein are isolated nucleic acid molecules comprising a sequence encoding an AAV rep protein, wherein the isolated nucleic acid molecule does not comprise a sequence encoding a capsid.

In some embodiments, the AAV rep protein comprises REP2. In some embodiments, the isolated nuclei acid molecule further comprises one or more helper elements. In some embodiments, the one or more helper elements comprises Ad5-VA, Ad5-E2A(DBP), or Ad5-E4. In some embodiments, the isolated nuclei acid molecule further comprises a p5 promoter 5′ of the sequence encoding the AAV rep protein. In some embodiments, the isolated nucleic acid molecule further comprises a sequence encoding a non-AUG translation start codon 5′ of the sequence encoding the AAV rep protein. In some embodiments, the non-AUG translation start codon is ACG.

Disclosed herein are compositions comprising (a) a first nucleic acid molecule comprising i) a sequence encoding an AAV rep protein or ii) a sequence comprising one or more helper elements; and (b) a second nucleic acid molecule comprising i) sequence encoding an AAV capsid and ii) an exogenous gene flanked by inverted terminal repeat (ITR) sequences, wherein the first nucleic acid and the second nucleic acid are separate nucleic acid molecules.

In some embodiments, the first nucleic acid molecule comprises the sequence encoding the AAV rep protein and the sequence comprising the one or more helper elements. In some embodiments, the one or more helper elements comprises Ad5-VA, Ad5-E2A(DBP), or Ad5-E4. In some embodiments, the AAV rep protein comprises REP2. In some embodiments, the AAV capsid comprises a capsid from AAV1, AAV2, AAV2 variant, AAV3A, AAV3B, AAV4, AAV5, AAV6, AAV7, AAV8, AAV9, AAV9 variant, AAV10, AAV11, AAV12, AAV13, AAV-Rh10, AAV-Rh74, AAV-2i8, AAV-DJ, LK03, NP59, or KP1. In some embodiments, the exogeneous gene has a size from about 2 kilobases to about 7 kilobases. In some embodiments, the first nucleic acid molecule comprises a sequence with at least 70%, 75%, 80%, 85%, 90%, 95%, 96%, 97%, 98%, 99%, or 100% sequence identity of the sequence as set forth in SEQ ID NO: 2 or 3. In some embodiments, the second nucleic acid molecule comprises a sequence with at least 70%, 75%, 80%, 85%, 90%, 95%, 96%, 97%, 98%, 99%, or 100% sequence identity of the sequence as set forth in SEQ ID NO: 4, 5, 15, or 17. In some embodiments, the second nucleic acid molecule comprises a sequence with at least 70%, 75%, 80%, 85%, 90%, 95%, 96%, 97%, 98%, 99%, or 100% sequence identity of the sequence as set forth in SEQ ID NO: 14 or 18. In some embodiments, the first nucleic acid molecule comprises a sequence with at least 70%, 75%, 80%, 85%, 90%, 95%, 96%, 97%, 98%, 99%, or 100% sequence identity of the sequence as set forth in SEQ ID NO: 2, and wherein the second nucleic acid molecule comprises a sequence with at least 70%, 75%, 80%, 85%, 90%, 95%, 96%, 97%, 98%, 99%, or 100% sequence identity of the sequence as set forth in SEQ ID NO: 15. In some embodiments, the first nucleic acid molecule comprises a sequence with at least 70%, 75%, 80%, 85%, 90%, 95%, 96%, 97%, 98%, 99%, or 100% sequence identity of the sequence as set forth in SEQ ID NO: 2, and wherein the second nucleic acid molecule comprises a sequence with at least 70%, 75%, 80%, 85%, 90%, 95%, 96%, 97%, 98%, 99%, or 100% sequence identity of the sequence as set forth in SEQ ID NO: 17.

Disclosed herein are compositions comprising (a) a first isolated nucleic acid molecule comprising i) a sequence encoding an AAV rep protein or a sequence encoding an AAV capsid, and ii) an exogenous gene flanked by inverted terminal repeat (ITR) sequences; and (b) a second isolated nucleic acid molecule comprising (A) one or more helper elements, and (B) a sequence encoding an AAV capsid complementing the AAV rep protein in (a) or a sequence encoding an AAV rep protein complementing the AAV capsid in (a); wherein the first nucleic acid molecule and the second nucleic acid molecule are separate nucleic acid molecules.

In some embodiments, the first nucleic acid molecule comprises the sequence encoding the AAV rep protein. In some embodiments, the AAV rep protein comprises REP2. In some embodiments, the first nucleic acid molecule comprises the sequence encoding the AAV capsid. In some embodiments, the first nucleic acid molecule comprises a sequence with at least 70%, 75%, 80%, 85%, 90%, 95%, 96%, 97%, 98%, 99%, or 100% sequence identity of the sequence as set forth in SEQ ID NO: 14. In some embodiments, the first nucleic acid molecule comprises a sequence with at least 70%, 75%, 80%, 85%, 90%, 95%, 96%, 97%, 98%, 99%, or 100% sequence identity of the sequence as set forth in SEQ ID NO: 18. In some embodiments, the second nucleic acid molecule comprises a sequence with at least 70%, 75%, 80%, 85%, 90%, 95%, 96%, 97%, 98%, 99%, or 100% sequence identity of the sequence as set forth in SEQ ID NO: 2. In some embodiments, the AAV capsid comprises a capsid from AAV1, AAV2, AAV2 variant, AAV3A, AAV3B, AAV4, AAV5, AAV6, AAV7, AAV8, AAV9, AAV9 variant, AAV10, AAV11, AAV12, AAV13, AAV-Rh10, AAV-Rh74, AAV-2i8, AAV-DJ, LK03, NP59, or KP1. In some embodiments, the exogeneous gene has a size from about 2 kilobases to about 7 kilobases. In some embodiments, the first nucleic acid molecule comprises the sequence encoding the AAV rep protein and the sequence encoding the AAV capsid. In some embodiments, the one or more helper elements comprises Ad5-VA, Ad5-E2A(DBP), or Ad5-E4. In some embodiments, the first isolated nucleic acid molecule comprises a sequence with at least 70%, 75%, 80%, 85%, 90%, 95%, 96%, 97%, 98%, 99%, or 100% sequence identity of the sequence as set forth in SEQ ID NO: 4. In some embodiments, the first isolated nucleic acid molecule comprises a sequence with at least 70%, 75%, 80%, 85%, 90%, 95%, 96%, 97%, 98%, 99%, or 100% sequence identity of the sequence as set forth in SEQ ID NO: 4, 5, 15 or 17. In some embodiments, the second isolated nucleic acid molecule comprises a sequence with at least 70%, 75%, 80%, 85%, 90%, 95%, 96%, 97%, 98%, 99%, or 100% sequence identity of the sequence as set forth in SEQ ID NO: 7.

Disclosed herein are cells comprising the isolated nucleic acid molecule or the composition disclosed herein. In some embodiments, the cell comprises a HEK293 cell. In some embodiments, the HEK293 cell comprises a suspension HEK293 cell.

Disclosed herein are methods comprising introducing the isolated nucleic acid molecule or the composition disclosed herein into a cell, thereby generating a cell comprising the isolated nucleic acid molecule or the composition. In some embodiments, the introducing comprises transient transfection. In some embodiments, the cell comprises a HEK293 cell. In some embodiments, the HEK293 cell comprises a suspension HEK293 cell.

Disclosed herein are methods of producing AAVs, the method comprising using the cell disclosed herein to generate the AAVs. In some embodiments, the generated AAVs comprise a full/empty ratio of at least 5%, 10%, 20%, 30%, 40%, or 50% prior to any purification steps. In some embodiments, a production yield of the generated AAVs is at least 1.1-fold, 1.5-fold, 2.0-fold, 2.5-fold, 3.0-fold, 3.5-fold, 4.0-fold, 4.5-fold, or 5.0-fold of a production yield of AAVs generated by a three-plasmid system.

Disclosed herein are compositions of AAVs generated by the method disclosed herein.

Disclosed herein are methods of treating a condition in a subject in need thereof comprising administering the composition of AAVs disclosed herein to the subject, thereby treating the condition.

Disclosed herein is use of the composition of AAVs disclosed herein in manufacture of a medicament for treating a condition in a subject in need thereof.

Disclosed herein are kits comprising the isolated nucleic acid molecule or the composition disclosed herein, and instructions.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-D illustrate a simplified scheme of the two-plasmid systems disclosed herein. FIG. 1A illustrates a simplified scheme of a two-plasmid system where one plasmid contains a nucleic acid sequence encoding rep and cap proteins (RC) and a gene of interest (GOI), while the other plasmid contains a helper element (Help). FIG. 1B illustrates a simplified scheme of a two-plasmid system where one plasmid contains a nucleic acid sequence encoding rep and cap proteins (RC) and a helper element (Help), while the other plasmid contains a gene of interest (GOI). FIG. 1C illustrates a simplified scheme of a two-plasmid system where one plasmid contains a nucleic acid sequence encoding a cap protein (C) and a gene of interest (GOI), while the other plasmid contains a nucleic acid sequence encoding a rep protein (R) and a helper element (Help). FIG. 1D illustrates a simplified scheme of a two-plasmid system where one plasmid contains a nucleic acid sequence encoding a rep protein (R) and a gene of interest (GOI), while the other plasmid contains a nucleic acid sequence encoding a cap protein (C) and a helper element (Help).

FIGS. 2A-F illustrate structures of plasmids used in two-plasmid systems disclosed herein. FIG. 2A provides a structure of Plasmid #11, which contains nucleic acid sequence encoding the rep protein and the cap protein (RC1) and the helper elements (Help). p5: promoter p5; ACG: alternative start codon; REP2: AAV2 rep; CAP2: AAV2 capsid; pA: poly(A); VA: VA gene; E2A(DBP): E2A gene; E4: E4 gene. FIG. 2B provides a structure of Plasmid #12, which contains nucleic acid sequence encoding the rep protein (Alt Start Rep) and nucleic acid sequence comprising the helper elements (Help). p5: promoter p5; ACG: alternative start codon; REP2: AAV2 rep; pA: poly(A); VA: VA gene; E2A(DBP): E2A gene; E4: E4 gene. FIG. 2C provides a structure of Plasmid #13, which contains nucleic acid sequence encoding the rep protein and the cap protein (RC2) and nucleic acid sequence comprising the helper elements (Help). p5: promoter p5; REP2: AAV2 rep; CAP2: AAV2 capsid; pA: poly(A); VA: VA gene; E2A(DBP): E2A gene; E4: E4 gene. FIG. 2D provides a structure of Plasmid #14, which contains nucleic acid sequence encoding the cap protein (Ef1α-Cap2) and nucleic acid sequence comprising the gene of interest (GOI). Ef1α: Ef1α promoter; CAP2: AAV2 capsid; pA: poly(A); ITR: inverted terminal repeats; CMV: CMV promoter; WPRE: Woodchuck Hepatitis Virus (WHP) Posttranscriptional Regulatory Element (WPRE). FIG. 2E provides a structure of Plasmid #15, which contains nucleic acid sequence encoding the rep protein and the cap protein (RC1) and nucleic acid sequence comprising the gene of interest (GOI). p5: promoter p5; ACG: alternative start codon; REP2: AAV2 rep; CAP2: AAV2 capsid; pA: poly(A); ITR: inverted terminal repeats; CMV: CMV promoter; WPRE: Woodchuck Hepatitis Virus (WHP) Posttranscriptional Regulatory Element (WPRE). FIG. 2F provides a structure of Plasmid #16, which contains nucleic acid sequence encoding the rep protein and the cap protein (RC2) and nucleic acid sequence comprising the gene of interest (GOI). p5: promoter p5; REP2: AAV2 rep; CAP2: AAV2 capsid; pA: poly(A); ITR: inverted terminal repeats; CMV: CMV promoter; WPRE: Woodchuck Hepatitis Virus (WHP) Posttranscriptional Regulatory Element (WPRE).

FIGS. 3A-F illustrate plasmid maps. FIG. 3A provides a map of Plasmid #11. FIG. 3B provides a map of Plasmid #12. FIG. 3C provides a map of Plasmid #13. FIG. 3D provides a map of Plasmid #14. FIG. 3E provides a map of Plasmid #15. FIG. 3F provides a map of Plasmid #16.

FIGS. 4A-B illustrates a schematic structure and map of a plasmid comprising helper elements including VA, E2A and E4. FIG. 4A provides a schematic structure of the plasmid comprising helper elements. VA: VA gene; E2A(DBP): E2A gene; E4: E4 gene. FIG. 4B provides a map of the plasmid comprising helper elements. The plasmid lacks an Ad5 Fiber sequence.

FIG. 5 demonstrates rAAV production using two-plasmid systems disclosed herein as compared to a three-plasmid control system (Control).

FIGS. 6A-B illustrate schematic structures of nucleic acid sequences encoding AAV rep protein and capsid. FIG. 6A provides a schematic stricture of a nucleic acid sequence encoding the AAV rep protein and capsid comprising a non-AUG start codon (i.e., ACG, Plasmid #03). FIG. 6B provides a schematic of a nucleic acid sequence encoding the AAV rep protein and capsid with p5 promoter downstream of the sequence encoding the capsid. ATG: start codon; ACG: alternative start codon; REP2: AAV2 rep; CAP: AAV capsid; p5: p5 promoter.

FIG. 7 illustrates a schematic structure of a plasmid comprising GOI without sequence encoding rep, capsid, or helper element (Plasmid #20). ITR: inverted terminal repeats; pA: poly(A); CMV: CMV promoter; WPRE: Woodchuck Hepatitis Virus (WHP) Posttranscriptional Regulatory Element (WPRE).

FIG. 8 provides a list of nucleic acid sequences disclosed herein.

FIG. 9 illustrates the structure of an exemplary plasmid (Plasmid #17) used in the two-plasmid systems disclosed herein. Plasmid #17 contains nucleic acid sequence encoding the cap protein (p40-Cap2) and nucleic acid sequence comprising the gene of interest (GOI). p40: AAV-p40 promoter; CAP2: AAV2 capsid; pA: poly(A); ITR: inverted terminal repeats; CMV: CMV promoter; WPRE: Woodchuck Hepatitis Virus (WHP) Posttranscriptional Regulatory Element (WPRE).

FIGS. 10A-B illustrate the plasmid map of Plasmid #17.

FIG. 11 demonstrates rAAV production using two-plasmid system disclosed herein (Plasmid #12+Plasmid #17) as compared to a three-plasmid control system (Control).

### DETAILED DESCRIPTION

Certain specific details of this description are set forth in order to provide a thorough understanding of various embodiments. However, one skilled in the art will understand that the present disclosure may be practiced without these details. In other instances, well-known structures have not been shown or described in detail to avoid unnecessarily obscuring descriptions of the embodiments.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods, and materials are described below. Further, headings provided herein are for convenience only and do not interpret the scope or meaning of the claimed disclosure. It is contemplated that any embodiment discussed in this specification can be implemented with respect to any method or composition of the present disclosure, and vice versa. Furthermore, compositions of the present disclosure can be used to achieve methods of the present disclosure.

### Definitions

To facilitate an understanding of the present disclosure, a number of terms and phrases are defined below.

As used in this specification and the appended claims, the singular forms “a,” “an,” and “the” include plural referents unless the content clearly dictates otherwise.

It should also be noted that the term “or” is generally employed in its sense including “and/or” unless the content clearly dictates otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, within 5-fold, and more preferably within 2-fold, of a value. Where particular values are described in the application and claims, unless otherwise stated the term “about” meaning within an acceptable error range for the particular value should be assumed.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, “some embodiments,” “an embodiment,” “one embodiment,” “embodiments” or “other embodiments” means that a particular feature, structure, or characteristic described in connection with the embodiments is included in at least some embodiments, but not necessarily all embodiments, of the present disclosures.

The terms “polynucleotide,” “nucleic acid,” and “oligonucleotide” are used interchangeably. They refer to a polymer containing at least two nucleotides (e.g., deoxyribonucleotides or ribonucleotides), e.g., in either single- or double-stranded form, and includes DNA and RNA, hybrids of DNA and RNA, and combinations thereof. The term “nucleic acid” as used herein also refers to a polymer containing at least two chemically modified nucleotides (e.g., deoxyribonucleotides or ribonucleotides), e.g., in either single- or double-stranded form and includes DNA and RNA, hybrids of DNA and RNA, and combinations thereof.

Unless otherwise indicated, a particular nucleic acid sequence encoding a protein or a peptide can also encompass conservatively modified variants thereof (e.g., degenerate codon substitutions), alleles, orthologs, SNPs, and complementary sequences that encode the same protein or peptide. Specifically, degenerate codon substitutions can be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mol. Cell. Probes, 8:91-98 (1994)).

The term “gene” can refer to a polynucleotide containing at least one open reading frame, while the open reading frame encodes a particular protein. Gene can be a group of genes, a cDNA, or a synthetic nucleic acid (e.g., synthetic DNA or RNA).

As used herein, the terms “GOI,” “gene of interest,” and “exogenous gene” are interchangeable. These terms can refer to a gene that is introduced, or is to be introduced, to a particular cell by external means (e.g., expression vectors). A GOI can be a variant (e.g., a mutant) of a gene normally expressed in a particular cell. A GOI can be the same gene that is normally expressed in a particular cell.

As used herein, the term “regulatory element” can refer to a genetic element which controls some aspect of the expression of nucleic acid sequences. For example, a promoter can be a regulatory element which facilitates the initiation of transcription of an operably linked coding region. Other regulatory elements can be splicing signals, polyadenylation signals, termination signals, etc.

The term “control sequences” can refer collectively to regulatory elements such as promoter sequences, polyadenylation signals, transcription termination sequences, upstream regulatory domains, origins of replication, internal ribosome entry sites (“IRES”), enhancers, and the like, which collectively provide for the replication, transcription and translation of a coding sequence in a recipient cell. Not all of these control sequences need always be present so long as the selected coding sequence is capable of being replicated, transcribed and translated in an appropriate recipient cell.

Transcriptional control sequences in eukaryotes can comprise “promoter” and “enhancer” elements. Promoters and enhancers can comprise short arrays of DNA sequences that interact specifically with cellular proteins involved in transcription (Maniatis et al., Science 236:1237 [1987]). Promoter and enhancer elements have been isolated from a variety of eukaryotic sources including genes in yeast, insect and mammalian cells and viruses (analogous control sequences, i.e., promoters, are also found in prokaryotes). The selection of a particular promoter and enhancer depends on what cell type is to be used to express the protein of interest (i.e., Factor VIII). Some eukaryotic promoters and enhancers have a broad host range while others are functional in a limited subset of cell types (See e.g., Voss et al., Trends Biochem. Sci., 11:287 [1986]; and Maniatis et al., supra, for reviews). For example, the SV40 early gene enhancer can be active in a wide variety of cell types from many mammalian species and has been widely used for the expression of proteins in mammalian cells (Dijkema et al., EMBO J. 4:761 [1985]). Two other examples of promoter and enhancer elements active in a broad range of mammalian cell types are those from the human elongation factor lax gene (Uetsuki et al., J. Biol. Chem., 264:5791 [1989]; Kim et al., Gene 91:217 [1990]; and Mizushima and Nagata, Nucl. Acids. Res., 18:5322 [1990]) and the long terminal repeats of the Rous sarcoma virus (Gorman et al., Proc. Natl. Acad. Sci. USA 79:6777 [1982]) and the human cytomegalovirus (Boshart et al., Cell 41:521 [1985]). Promoters and enhances can be found naturally alone or together. For example, the long terminal repeats of retroviruses can contain both promoter and enhancer functions. Moreover, generally promoters and enhances can act independently of the gene being transcribed or translated. Thus, the enhancer and promoter can be “endogenous” or “exogenous” or “heterologous.” An “endogenous” enhancer/promoter can be one which is naturally linked with a given gene in the genome. An “exogenous” or “heterologous” enhancer and promoter can be one which is placed in juxtaposition to a gene by means of genetic manipulation (i.e., molecular biological techniques) such that transcription of that gene is directed by the linked enhancer/promoter.

As used herein, the term “tissue specific” can refer to control sequences, such as a promoter, enhancers, etc., wherein the expression of the nucleic acid sequence is substantially greater in a specific cell type(s) or tissue(s).

“Operably linked” can refer to an arrangement of elements wherein the components so described are configured so as to perform their usual function. Thus, control sequences operably linked to a coding sequence can be capable of effecting the expression of the coding sequence. The control sequences need not be contiguous with the coding sequence, so long as they function to direct the expression thereof. Thus, for example, intervening untranslated yet transcribed sequences can be present between a promoter sequence and the coding sequence and the promoter sequence can still be considered “operably linked” to the coding sequence.

As used herein, the term “vector” can refer to any genetic element, such as a plasmid, phage, transposon, cosmid, chromosome, virus, virion, etc., which is capable of replication when associated with the proper regulatory elements and/or which can transfer nucleic acid sequences between cells. Thus, the term can include cloning and expression vectors, as well as viral vectors.

Vectors can include transcription sequences such as polyadenylation sites, selectable markers or reporter genes, enhancer sequences, and other regulatory elements which allow for the induction of transcription. Such regulatory elements are described more fully below.

The term “expression vector” as used herein can refer to a recombinant DNA molecule containing a desired coding sequence and appropriate nucleic acid sequences for the expression of the operably linked coding sequence in a particular host organism. Nucleic acid sequences for expression in prokaryotes can include a promoter, an operator (optional), and a ribosome binding site, as well as other sequences. Eukaryotic cells can utilize promoters (constitutive, inducible or tissue specific), enhancers, and termination and polyadenylation signals, although some elements can be deleted and other elements added without sacrificing the expression.

As used herein, the terms “host” can refer to organisms and/or cells which harbor an exogenous DNA sequence (e.g., via transfection), an expression vector or vehicle, as well as organisms and/or cells that are suitable for use in expressing a recombinant gene or protein.

As used herein, the terms “protein,” “polypeptide,” and “peptide” are used interchangeably and can refer to a polymer of amino acid residues linked via peptide bonds and which can be composed of two or more polypeptide chains. The terms “polypeptide,” “protein,” and “peptide” can refer to a polymer of at least two amino acid monomers joined together through amide bonds. An amino acid can be the L-optical isomer or the D-optical isomer. The terms “polypeptide,” “protein,” and “peptide” can refer to a molecule composed of two or more amino acids in a specific order; for example, the order as determined by the base sequence of nucleotides in the gene or RNA coding for the protein. Examples are hormones, enzymes, antibodies, and any fragments thereof. A protein can be a portion of the protein, for example, a domain, a subdomain, or a motif of the protein. A protein can be a variant (or mutation) of the protein, wherein one or more amino acid residues are inserted into, deleted from, and/or substituted into the naturally occurring (or at least a known) amino acid sequence of the protein. A protein or a variant thereof can be naturally occurring or recombinant. Methods for detection and/or measurement of polypeptides in biological material can include Western-blotting, flow cytometry, ELISAs, RIAs, and various proteomics techniques. An exemplary method to measure or detect a polypeptide is an immunoassay, such as an ELISA. This type of protein quantitation can be based on an antibody capable of capturing a specific antigen, and a second antibody capable of detecting the captured antigen. Exemplary assays for detection and/or measurement of polypeptides are described in Harlow, E. and Lane, D. Antibodies: A Laboratory Manual, (1988), Cold Spring Harbor Laboratory Press.

The term “sequence identity,” as used herein, can refer to the amount of nucleotide or amino acid which match exactly between two different sequences. Sequence identity can be determined by standard alignment algorithm programs used with default gap penalties established by each supplier. For example, the BLAST function of the National Center for Biotechnology Information database can be used to determine identity.

A “subject” in need thereof, can refer to an individual who has a disease, a symptom of the disease, or a predisposition toward the disease, with the purpose to cure, heal, alleviate, relieve, alter, remedy, ameliorate, improve, or affect the disease, the symptom of the disease, or the predisposition toward the disease.

The terms “treat,” “treating,” or “treatment,” and its grammatical equivalents as used herein, can include alleviating, abating, or ameliorating at least one symptom of a disease or a condition, preventing additional symptoms, inhibiting the disease or the condition, e.g., delaying, decreasing, suppressing, attenuating, diminishing, arresting, or stabilizing the development or progression of a disease or the condition, relieving the disease or the condition, causing regression of the disease or the condition, relieving a condition caused by the disease or the condition, reducing disease severity, or stopping the symptoms of the disease or the condition either prophylactically and/or therapeutically. “Treating” can also include lessening the frequency of occurrence or recurrence, or the severity, of any symptoms or other ill effects related to a disease or condition and/or the side effects associated with the disease or condition. “Treating” does not necessarily require curative results. It is appreciated that, although not precluded, treating a disorder or condition also does not require that the disorder, condition, or symptoms associated therewith be completely eliminated. The term “treating” encompasses the concept of “managing” which refers to reducing the severity of a particular disease or disorder in a patient or delaying its recurrence, e.g., lengthening the period of remission in a patient who had suffered from the disease. “Treating” can refer to the application or administration or a composition to a subject after the onset, or suspected onset, of a disease or condition.

The term “treating” further encompasses the concept of “prevent,” “preventing,” and “prevention.” The terms “prevent,” “preventing,” and “prevention,” as used herein, can refer to a decrease in the occurrence of pathology of a condition in a subject, who does not have, but is at risk of or susceptible to developing a disease or condition. The prevention can be complete, e.g., the total absence of pathology of a condition in a subject. The prevention can also be partial, such that the occurrence of pathology of a condition in a subject is less than that which would have occurred without the present disclosure.

“Administering” and its grammatical equivalents as used herein can refer to providing pharmaceutical compositions described herein to a subject or a patient. Different methods can be used to administer the composition to the subject, depending upon the type of disease to be treated or the site of the disease. For example, the composition can be administered, e.g., orally, parenterally, by inhalation spray, topically, rectally, nasally, buccally, vaginally, via an implanted reservoir, or via infusion. One or more such routes can be employed.

The terms “pharmaceutical composition” and its grammatical equivalents as used herein can refer to a mixture or solution comprising a therapeutically effective amount of an active pharmaceutical ingredient together with one or more pharmaceutically acceptable excipients, carriers, and/or a therapeutic agent to be administered to a subject, e.g., a human in need thereof.

The term “pharmaceutically acceptable” and its grammatical equivalents as used herein can refer to an attribute of a material which is useful in preparing a pharmaceutical composition that is generally safe, non-toxic, and neither biologically nor otherwise undesirable and is acceptable for veterinary as well as human pharmaceutical use. “Pharmaceutically acceptable” can refer a material, such as a carrier or diluent, which does not abrogate the biological activity or properties of the compound, and is relatively nontoxic, i.e., the material can be administered to a subject without causing undesirable biological effects or interacting in a deleterious manner with any of the components of the pharmaceutical composition in which it is contained.

A “pharmaceutically acceptable excipient, carrier, or diluent” can refer to an excipient, carrier, or diluent that can be administered to a subject, together with an agent, and which does not destroy the pharmacological activity thereof and is nontoxic when administered in doses sufficient to deliver a therapeutic amount of the agent.

The term “therapeutic agent” can refer to any agent that, when administered to a subject, has a therapeutic, diagnostic, and/or prophylactic effect and/or elicits a desired biological and/or pharmacological effect.

A “therapeutically effective amount” as used herein can refer to the amount of each composition of the present disclosure that confers therapeutic effect on the subject, either alone or in combination with one or more other therapeutic agents. Hence, as used herein, the term “therapeutically effective amount” can mean an amount of an agent to be delivered (e.g., nucleic acid, composition, therapeutic agent, prophylactic agent, etc.) that is sufficient, when administered to a subject suffering from or susceptible to a disease, disorder, and/or condition, to treat, improve symptoms of, diagnose, prevent, and/or delay the onset of the disease, disorder, and/or condition. In terms of treatment, a “therapeutically effective amount” can be an amount that is sufficient to palliate, ameliorate, stabilize, reverse or slow the progression of a disease or a condition, e.g., an atherosclerotic vascular disease, hypertriglyceridemia, or diabetes. A “therapeutically effective amount” can vary, as recognized by those skilled in the art, depending on the particular condition being treated, the severity of the condition, the individual subject parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. In some cases, a maximum dose of the individual components or combinations thereof be used, that is, the highest safe dose according to sound medical judgment. In some cases, a subject may insist upon a lower dose or tolerable dose for medical reasons, psychological reasons or for virtually any other reasons. Additionally, other medication the patient may be receiving can affect the determination of the therapeutically effective amount of the therapeutic agent to administer. Empirical considerations, such as the half-life, generally can contribute to the determination of the dosage. A “therapeutically effective amount” can be of any of the compositions of the disclosure used alone or in conjunction with one or more agents used to treat a condition. A therapeutically effective amount can be administered in one or more administrations.

As used herein, the term “AAV vector” can refer to a vector having functional or partly functional ITR sequences. The ITR sequences can be an ITR sequence from an adeno-associated virus serotype including, but not limited to, AAV serotype 1 (AAV1), AAV serotype 2 (AAV2), AAV2 variants, AAV serotype 3 (AA. V3, including serotypes 3A and 3B), AAV serotype 4 (AAV4), AAV serotype 5 (AAV5), AAV serotype 6 (AAV6), AAV serotype 7 (AAV7), AAV serotype 8 (AAV8), AAV serotype 9 (AAV9), AAV serotype 10 (AAV10), AAV serotype 11 (AAV11), AAV serotype 12 (AAV12), AAV serotype 13 (AAV13), AAV-Rh10, AAV-Rh74, AAV-2i8 or any other AAVs. The ITRs, however, need not be the wild-type nucleotide sequences, and can be altered (e.g., by the insertion, deletion or substitution of nucleotides), so long as the sequences retain function provide for functional rescue, replication and packaging. AAV vectors can have one or more of the AAV wild-type genes deleted in whole or part, e.g., the rep and/or cap genes but retain functional flanking ITR sequences. Functional ITR sequences can be used for the rescue, replication and packaging of the AAV particles.

As used herein, the term “ITR” refers to inverted terminal repeats. The terms “adeno-associated virus inverted terminal repeats” or “AAV ITRs” can refer to the palindromic regions found at each end of the AAV genome which can function together in cis as origins of DNA replication and as packaging signals for the virus. For example, flanking AAV ITRs can be positioned 5′ and 3′ of one or more heterologous nucleotide sequences (e.g., a GOI).

As used herein, the term “AAV rep protein” or “rep protein” can refer to the AAV replication proteins of Rep 78, Rep 68, Rep 52 and Rep 40. These Rep proteins can have many functions, including recognition, binding and nicking of the AAV origin of DNA replication, DNA helicase activity and modulation of transcription from AAV (or other heterologous) promoters. The Rep proteins can be used collectively for replicating the AAV genome. Muzyczka (Muzyczka, Curr. Top. Microbiol. Immunol., 158:97-129 [1992]) and Kotin (Kotin, Hum. Gene Ther., 5:793-801 [1994]) provide additional descriptions of the coding region for AAV rep proteins. Suitable AAV proteins can be from AAV2 or AAV5.

As used herein, the term “AAV cap protein,” “AAV capsid,” or “capsid” can refer to the AAV capsid proteins VP1, VP2, and VP3, or functional homologues thereof. These cap proteins can supply the packaging functions which can be collectively use for packaging the viral genome.

As used herein, the terms “helper functions” and “helper factors” can refer to functions and factors that are used by AAV for replication, but are not provided by the AAV particle (or rAAV particle) itself. Thus, these helper functions and factors can be provided by the host cell, a virus (e.g., adenovirus (Ad), herpes simplex virus (HSV), or human papilloma virus (HPV)), or another expression vector that is co-expressed in the same cell. The E1, E2A, E4 and VA coding regions of adenovirus can be used to supply the helper function for AAV replication and packaging (Matsushita et al., Gene Therapy 5:938 [1998]).

As used herein, the term “helper element” can refer to the coding regions encoding a helper factor having the helper functions. For example, the E1, E2A, E4 and VA coding regions of adenovirus. In some cases, the helper element is one from Ad2 or Ad5.

As used herein, the term “wild type” (“wt”) can refer to a gene or gene product which has the characteristics of that gene or gene product when isolated from a naturally occurring source. A wild-type gene is that which is most frequently observed in a population and is thus arbitrarily designed the “normal” or “wild-type” form of the gene. In contrast, the term “modified” or “mutant” can refer to a gene or gene product which displays modifications in sequence and or functional properties (i.e., altered characteristics) when compared to the wild-type gene or gene product. It is noted that naturally-occurring mutants can be isolated; these are identified by the fact that they have altered characteristics when compared to the wild-type gene or gene product.

As used herein, the term “AAV” or “AAV particle” can refer to a complete virus particle, such as a “wild-type” (wt) AAV virus particle (comprising a linear, single-stranded AAV nucleic acid genome associated with an AAV capsid protein coat). In this regard, single-stranded AAV nucleic acid molecules of either complementary sense (e.g., “sense” or “antisense” strands), can be packaged into any one AAV particle and both strands are equally infectious.

As used herein, the terms “recombinant AAV particle,” and “rAAV particle” can refer to an infectious viral particle containing a GOI (e.g., human growth hormone (hGH) sequence) which is flanked on both sides by AAV ITRs. In some cases, an rAAV particle is produced in a suitable host cell which contains an AAV vector and AAV helper functions introduced therein. In this manner, the host cell can be rendered capable of encoding AAV polypeptides for packaging the AAV vector containing a recombinant nucleotide sequence of interest, such as at least a portion of hGH, into recombinant particles for subsequent gene delivery.

An “AAV variant” as used herein refers to a viral particle comprising a variant AAV capsid. A “variant AAV capsid” refers to a capsid with at least one amino acid difference (e.g., amino acid substitution, amino acid insertion, amino acid deletion) relative to a corresponding parental AAV capsid. The variant capsid protein can confer the same or different infectivity of a host cell compared to the corresponding parental AAV capsid. The corresponding parental AAV capsid can be a naturally occurring AAV capsid.

The term “heterologous” can relate to nucleic acid sequences such as coding sequences and regulatory elements that are not normally joined together, and/or are not normally associated with a particular cell. Thus, a “heterologous” region of a nucleic acid construct or a vector can be a segment of nucleic acid within or attached to another nucleic acid molecule that is not found in association with the other molecule in nature. For example, a heterologous region of a nucleic acid construct can include a coding sequence flanked by sequences not found in association with the coding sequence in nature. Another example of a heterologous coding sequence is a construct where the coding sequence itself is not found in nature (e.g., synthetic sequences having codons different from the native gene). Similarly, a cell transfected with a construct which is not normally present in the cell can be considered heterologous for purposes of this disclosure.

As used herein, “coding sequence” or a sequence which “encodes” a particular protein or peptide can be a nucleic acid sequence which is transcribed (in the case of DNA) and translated (in the case of mRNA) into a polypeptide in vitro or in vivo, when placed under the control of appropriate regulatory elements. The boundaries of the coding sequence can be determined by a translation start codon at the 5′ (amino) terminus and a translation stop codon at the 3′ (carboxy) terminus. A coding sequence can include cDNA from prokaryotic or eukaryotic mRNA, genomic DNA sequences from prokaryotic or eukaryotic DNA, and even synthetic DNA sequences. A transcription termination sequence can be located 3′ to the coding sequence.

As used herein, the term “serotype” can refer to a distinguishable strain of a microorganism. A serotype can be a group of organisms that have the same type and number of surface antigens. Serotypes may or may not differ from strains, which can be isolates of a single culture. Serotypes may or may not differ from genotypes which have different sets of genes.

As used herein, the term “full/empty ratio” can refer to the ration between the number of the full rAAV particles and the number of the empty rAAV particles. The term “full rAAV particle,” as used herein, can refer to an rAAV particle containing the exogenous gene. The term “empty rAAV particle,” as used herein, can refer to an rAAV particle lacking the exogenous gene. The full/empty ratio can be related to impurity measurement of the rAAV production. The higher the full/empty ratio is, the better the purity of the rAAV product is. The full/empty ratio can be measured by multiple methods, including, but not limited to, single measurement techniques, Mass Photometry, Charge Detection Mass Spectroscopy (CD-MS), and Analytical Ultracentrifugation (AUC), and a Capsid Titer to Genome Titer ratio: Capsid Titer Measurement (measured by ELISA, Multi-Angle Light Scattering (MALS)) over the genome titer concentration (measured by PCR).

rAAV Production Systems

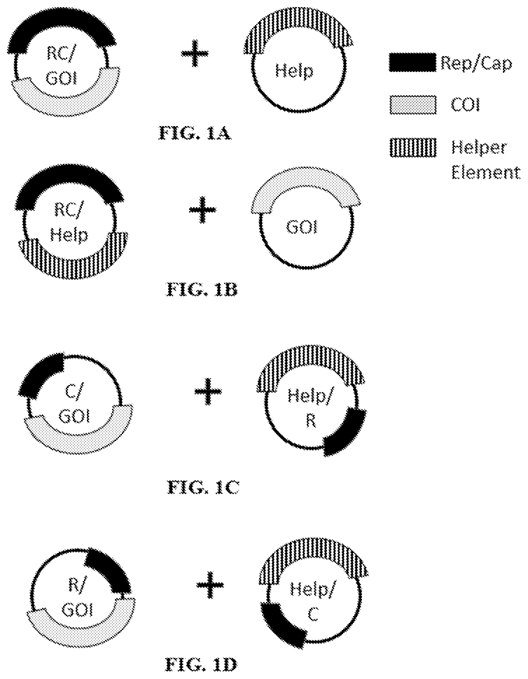

Disclosed herein are vector systems, e.g., two-vector (e.g., two-plasmid) systems for rAAV packaging. FIGS. 1A-D, show examples of four different two-plasmid systems for the improved rAAV production platform. One of the two-plasmid systems is illustrated in FIG. 1A, comprising (A) a first vector comprising (i) a first nucleic acid sequence comprising a sequence encoding an AAV rep protein and a sequence encoding an AAV capsid (RC) and (ii) a second nucleic acid sequence comprising an exogenous gene of interest (GOI), and (B) a second vector comprising one or more helper element (Help). The terms “exogenous gene of interest,” “exogenous gene,” “gene of interest,” and “GOI” are used interchangeably herein. Another one of the two-plasmid systems is illustrated in FIG. 1B, comprising (A) a first vector comprising (i) a first nucleic acid sequence comprising a sequence encoding an AAV rep protein and a sequence encoding an AAV capsid (RC) and (ii) a second nucleic acid sequence comprising one or more helper elements (Help), and (B) a second vector comprising a nucleic acid sequence comprising an exogenous gene of interest (GOI). Another two-plasmid system is illustrated in FIG. 1C, comprising (A) a first vector comprising (i) a first nucleic acid sequence encoding an AAV capsid (C) and (ii) a second nucleic acid sequence comprising an exogenous gene of interest (GOI), and (B) a second vector comprising (i) a first nucleic acid sequence encoding an AAV rep protein (R) and (ii) a second nucleic acid sequence comprising one or more helper elements (Help). Another two-plasmid system is illustrated in FIG. 1D, comprising (A) a first vector comprising (i) a first nucleic acid sequence encoding an AAV rep protein (R) and (ii) a second nucleic acid sequence comprising an exogenous gene of interest (GOI), and (B) a second vector comprising (i) a first nucleic acid sequence encoding an AAV capsid (C) and (ii) a second nucleic acid sequence comprising one or more helper elements (Help). By using the two-plasmid system, rAAV production can be achieved more efficiently by reducing the number of manufactured plasmid DNA components. The two-plasmid system also provides an improved transfection efficiency of genetic components by combining elements to improve productivity of a production platform of full AAV particles, and higher full/empty ratio of AAV particles produced from cell culture (i.e., less impurities).

rAAV Vectors

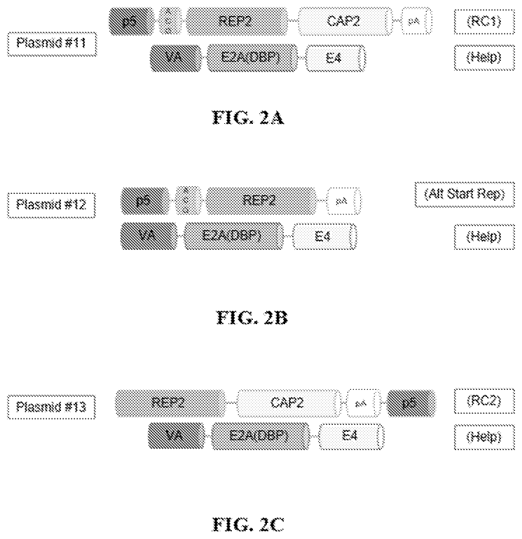

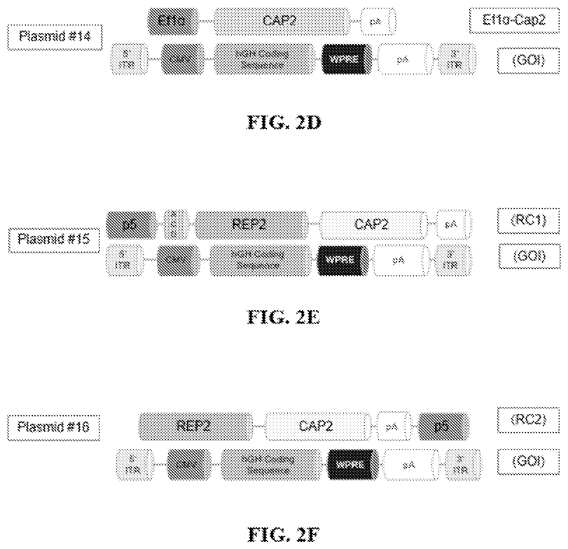

In some cases, a rAAV vector provided herein comprises a) a sequence encoding an adeno-associated virus (AAV) rep protein or a sequence encoding an AAV capsid, without a p5 promoter upstream of the sequence encoding the AAV rep protein or the sequence encoding an AAV capsid; and; and b) an exogenous gene flanked by inverted terminal repeat (ITR) sequences (e.g., Plasmid #16; see e.g., FIG. 2F). In some cases, the sequence encoding the AAV rep protein or the sequence the AAV capsid comprises a promoter downstream of the sequence encoding the AAV rep protein or the sequence encoding the AAV capsid. In some cases, the sequence encoding the AAV rep protein or the sequence the AAV capsid comprises a poly(A) sequence that is operably linked to the 3′ end of the sequence encoding the AAV rep protein or the sequence encoding the AAV capsid. In some cases, the promoter downstream of the sequence encoding the AAV rep protein or the sequence encoding the AAV capsid is operably linked to the 3′ end of the poly(A) sequence. In some cases, the promoter downstream of the sequence encoding the AAV rep protein or the sequence encoding the capsid is a p5 promoter. In some cases, the promoter is a p19 promoter. In some cases, the AAV rep protein is any AAV rep protein disclosed herein, e.g., AAV2 rep protein. In some cases, the AAV rep protein is encoded by REP2 gene which expresses Rep78/68 and Rep52/40. In some cases, the AAV capsid is any AAV capsid disclosed herein, e.g., is a capsid protein from AAV serotype 1 (AAV1), AAV serotype 2 (AAV2), AAV2 variants, AAV serotype 3 (AAV3, including serotypes 3A and 3B), AAV serotype 4 (AAV4), AAV serotype 5 (AAV5), AAV serotype 6 (AAV6), AAV serotype 7 (AAV7), AAV serotype 8 (AAV8), AAV serotype 9 (AAV9), AAV9 variants, AAV serotype 10 (AAV10), AAV serotype 11 (AAV11), AAV serotype 12 (AAV12), AAV serotype 13 (AAV13), AAV-Rh10, AAV-Rh74, AAV-2i8, AAV-DJ, LK03, NP59, or KP1, or any other AAVs. In some cases, the rAAV vector comprises, as operatively linked components, a first nucleic acid sequence comprising an AAV rep protein coding sequence, a AAV capsid coding sequence, a poly(A) sequence, and a promoter, wherein the first nucleic acid sequence lacks a p5 promoter at the 5′ end of the rep protein coding sequence; and a second nucleic acid sequence comprising a 5′ ITR, a promoter, a GOI coding sequence, a WPRE, a poly(A) sequence, and a 3′ ITR. In some cases, the promoter in the second nucleic acid sequence is a CMV promoter. In some cases, the rAAV vector comprises a nucleic acid sequence with about 70%, about 75%, about 80%, about 85%, about 90%, about 95%, about 96%, about 97%, about 98%, about 99%, about 99.5%, or about 99.9% sequence identity to SEQ ID NO: 5. In some cases, the rAAV vector comprises a nucleic acid sequence with at least 70%, at least 75%, at least 80%, at least 85%, at least 90%, at least 95%, at least 96%, at least 97%, at least 98%, at least 99%, at least 99.5%, or at least 99.9% sequence identity to SEQ ID NO: 5. In some cases, the rAAV vector comprises a sequence of SEQ ID NO: 5. In some cases, the rAAV vector consists of a nucleic acid sequence with about 70%, about 75%, about 80%, about 85%, about 90%, about 95%, about 96%, about 97%, about 98%, about 99%, about 99.5%, or about 99.9% sequence identity to SEQ ID NO: 5. In some cases, the rAAV vector consists of a nucleic acid sequence with at least 70%, at least t 75%, at least 80%, at least 85%, at least 90%, at least 95%, at least 96%, at least 97%, at least 98%, at least 99%, at least 99.5%, or at least 99.9% sequence identity to SEQ ID NO: 5. In some cases, the rAAV vector consists of a sequence of SEQ ID NO: 5.

In some cases, a rAAV vector provided herein comprises a) a sequence encoding an AAV rep protein or a sequence encoding an AAV capsid, without a p5 promoter downstream of the sequence encoding the AAV rep protein or the sequence encoding an AAV capsid; and b) an exogenous gene flanked by inverted terminal repeat (ITR) sequences (e.g., Plasmid #15; see e.g., FIG. 2E). In some cases, the rAAV vector comprises the sequence encoding the AAV rep protein. In some cases, the rAAV vector further comprises a sequence encoding a non-AUG translation start codon 5′ of the sequence encoding the AAV rep protein and the sequence encoding the AAV capsid. In some cases, the non-AUG translation start codon is CUG, GUG, UUG, ACG, AUC, AUU, AAG, AUA, or AGG. In some cases, the non-AUG translation start codon is ACG. In some cases, the non-AUC translation start codon is upstream of the sequence encoding the AAV rep protein and the sequence encoding the AAV capsid. In some cases, the non-AUC start codon is at 5′ end of the rep protein coding sequence and the capsid coding sequence. In some cases, the sequence encoding the AAV rep protein or the sequence encoding the AAV capsid comprises a promoter upstream of the sequence encoding the rep protein. In some cases, the promoter is p5 promoter. In some cases, the sequence encoding the AAV rep protein or the sequence encoding the AAV capsid comprises a poly(A) sequence. The poly(A) sequence can be located at the 3′ end of the capsid coding sequence. The AAV rep protein can be any rep protein disclosed herein, e.g., AAV2 rep protein. The AAV capsid can be any capsid disclosed herein, e.g., AAV serotype 1 (AAV1), AAV serotype 2 (AAV2), AAV2 variants, AAV serotype 3 (AAV3, including serotypes 3A and 3B), AAV serotype 4 (AAV4), AAV serotype 5 (AAV5), AAV serotype 6 (AAV6), AAV serotype 7 (AAV7), AAV serotype 8 (AAV8), AAV serotype 9 (AAV9), AAV9 variants, AAV serotype 10 (AAV10), AAV serotype 11 (AAV11), AAV serotype 12 (AAV12), AAV serotype 13 (AAV13), AAV-Rh10, AAV-Rh74, AAV-2i8, AAV-DJ, LK03, NP59, or KP1, or any other AAVs capsid. In some cases, the exogenous gene further comprises a promoter, a post-transcriptional regulatory element, or a poly(A). In some cases, the promoter is a CMV promoter. In some cases, the post-transcriptional regulatory element is WPRE. In some cases, the rAAV vectors comprise, as operatively linked components, a first nucleic acid sequence comprising a p5 promoter, a non-AUG start codon, a rep protein coding sequence, a capsid coding sequence, and a poly(A) sequence; and a second nucleic acid sequence comprising a 5′ ITR, a promoter, a GOI coding sequence, a WPRE, a poly(A) sequence, and a 3′ ITR. In some cases, the promoter in the second nucleic acid sequence is a CMV promoter. In some cases, the non-AUG start codon is ACG.

In some cases, the rAAV vector comprises a nucleic acid sequence with about 70%, about 75%, about 80%, about 85%, about 90%, about 95%, about 96%, about 97%, about 98%, about 99%, about 99.5%, or about 99.9% sequence identity to SEQ ID NO: 4. In some cases, the rAAV vector comprises a nucleic acid sequence with at least 70%, at least 75%, at least 80%, at least 85%, 90%, at least 95%, at least 96%, at least 97%, at least 98%, at least 99%, at least 99.5%, or at least 99.9% sequence identity to SEQ ID NO: 4. In some cases, the rAAV vector comprises a sequence of SEQ ID NO: 4. In some cases, the rAAV vector consists of a nucleic acid sequence with about 70%, about 75%, about 80%, about 85%, about 90%, about 95%, about 96%, about 97%, about 98%, about 99%, about 99.5%, or about 99.9% sequence identity to SEQ ID NO: 4. In some cases, the rAAV vector consists of a nucleic acid sequence with at least 70%, at least 75%, at least 80%, at least 85%, at least 90%, at least 95%, at least 96%, at least 97%, at least 98%, at least 99%, at least 99.5%, or at least 99.9% sequence identity to SEQ ID NO: 4. In some cases, the rAAV vector consists of a sequence of SEQ ID NO: 4.

In some cases, the rAAV vectors comprise a) a sequence encoding an AAV capsid, wherein isolated nucleic acid molecule does not comprise a sequence encoding AAV rep; and b) an exogenous gene flanked by inverted terminal repeat (ITR) sequences (e.g., Plasmid #14; see e.g., FIG. 2D). In some cases, the sequence encoding the AAV capsid comprises a promoter upstream of the sequence encoding the AAV capsid. The promoter can be any strong constitutive promoter. In some cases, the promoter is Ef1α promoter. The promoter can be operably linked to the capsid coding sequence. The AAV capsid can be any capsid disclosed herein, e.g., AAV1, AAV2, AAV2 variants, AAV3 (including AAV3A and AAV3B), AAV4, AAV5, AAV6, AAV7, AAV8, AAV9, AAV9 variants, AAV10, AAV11, AAV12, AAV13, AAV-Rh10, AAV-Rh74, AAV-2i8, AAV-DJ, LK03, NP59, or KP1, or any other AAVs. In some cases, the AAV capsid is AAV2, AAV6, AAV7, AAV8, or AAV9 capsid. In some cases, the exogenous gene further comprises a promoter, a post-transcriptional regulatory element, or a poly(A) sequence. In some cases, the promoter is a CMV promoter. The promoter can be operably linked to the exogenous gene. In some cases, the post-transcriptional regulatory element is WPRE. The post-transcriptional regulatory element can be operably linked to the exogenous gene. In some cases, the rAAV vectors comprise, as operatively linked components, a first nucleic acid sequence comprising promoter, a capsid coding sequence, and a poly(A) sequence; and a second nucleic acid sequence comprising a 5′ ITR, a promoter, a GOI coding sequence, a WPRE, a poly(A) sequence, and a 3′ ITR. In some cases, the promoter in the first nucleic acid sequence is a Ef1α promoter. In some cases, the promoter in the second nucleic acid sequence is a CMV promoter.

In some cases, the rAAV vector comprises a nucleic acid sequence with about 70%, about 75%, about 80%, about 85%, about 90%, about 95%, about 96%, about 97%, about 98%, about 99%, about 99.5%, or about 99.9% sequence identity to SEQ ID NO: 15 In some cases, the rAAV vector comprises a nucleic acid sequence with at least 70%, at least 75%, at least 80%, at least 85%, at least 90%, at least 95%, at least 96%, at least 97%, at least 98%, at least 99%, at least 99.5%, or at least 99.9% sequence identity to SEQ ID NO: 15. In some cases, the rAAV vector comprises a sequence of SEQ ID NO: 15. In some cases, the rAAV vector consists of a nucleic acid sequence with about 70%, about 75%, about 80%, about 85%, about 90%, about 95%, about 96%, about 97%, about 98%, about 99%, about 99.5%, or about 99.9% sequence identity to SEQ ID NO: 15. In some cases, the rAAV vector consists of a nucleic acid sequence with at least 70%, at least 75%, at least 80%, at least 85%, at least 90%, at least 95%, at least 96%, at least 97%, at least 98%, at least 99%, at least 99.5%, or at least 99.9% sequence identity to SEQ ID NO: 15. In some cases, the rAAV vector consists of a sequence of SEQ ID NO: 15.

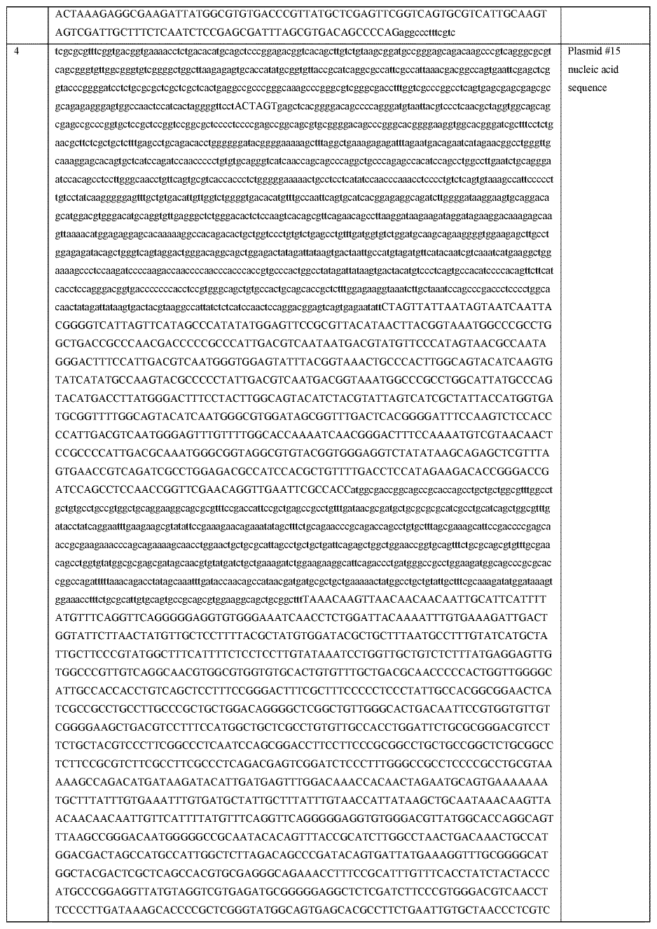

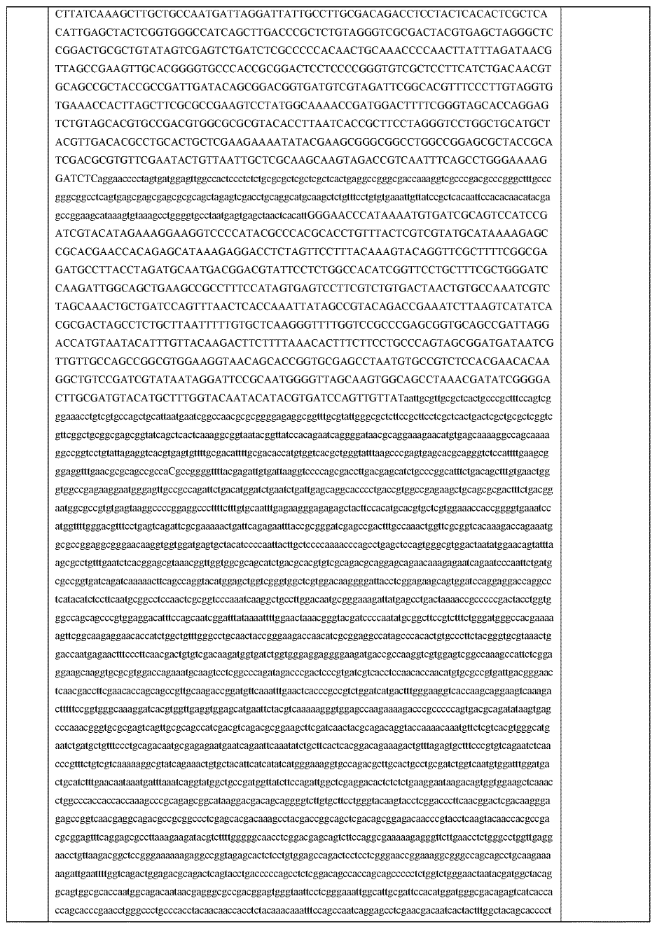

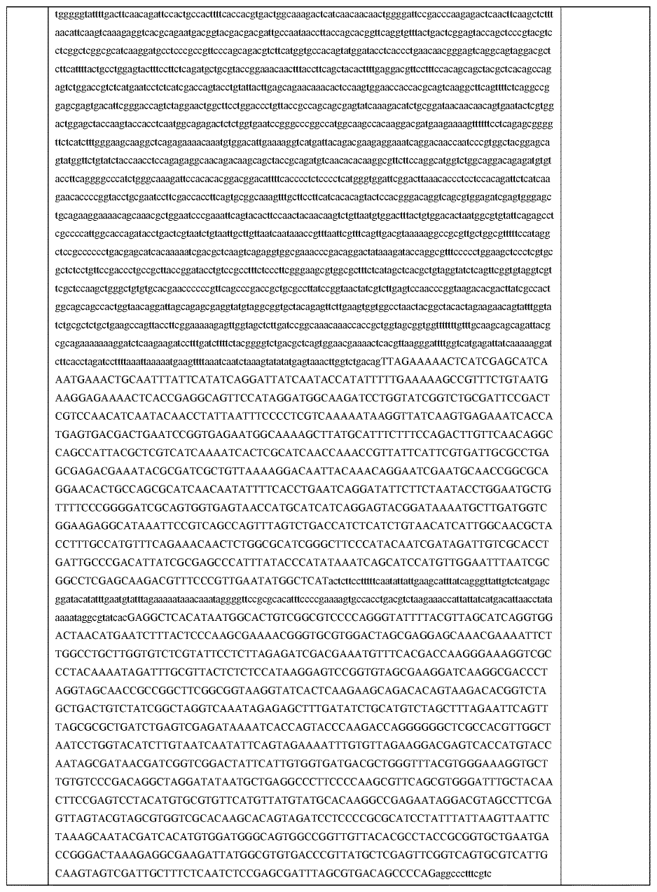

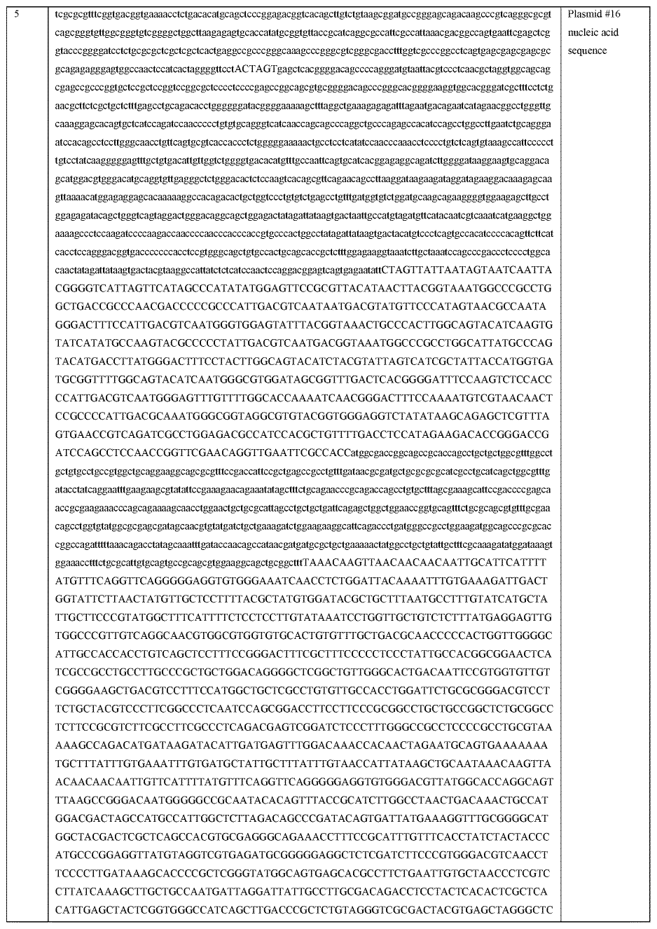

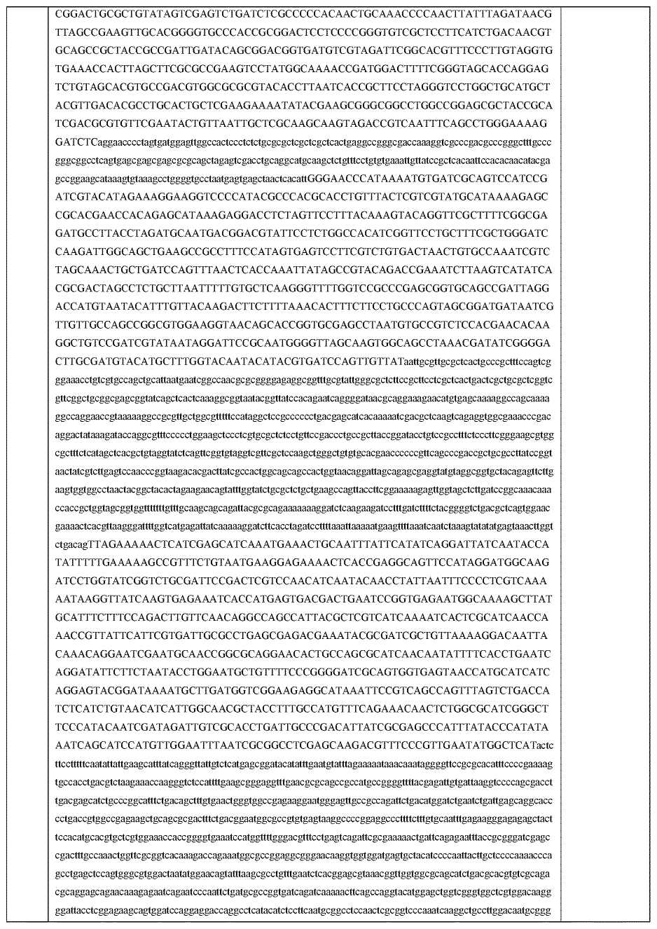

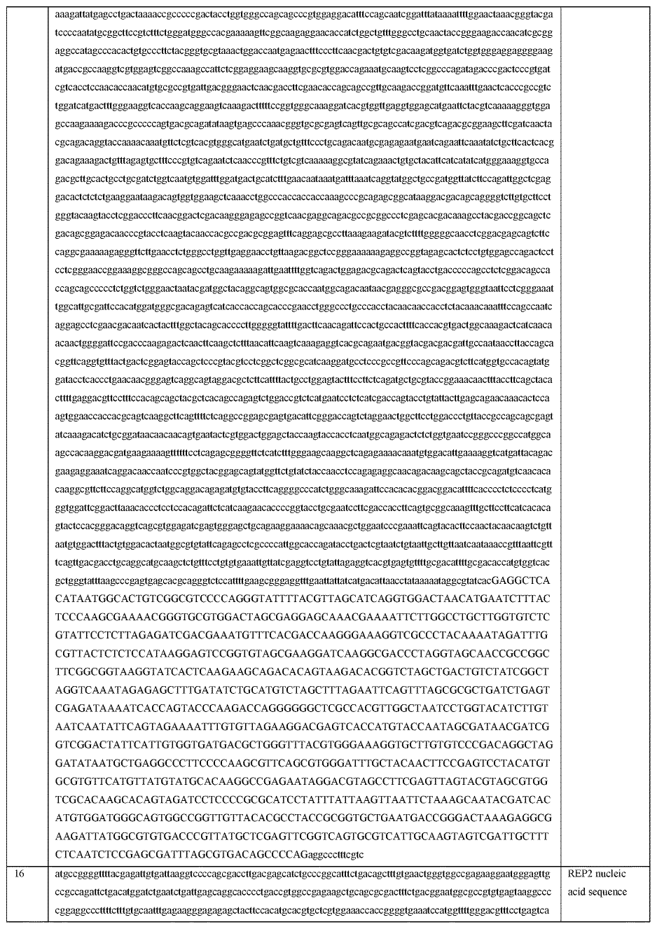

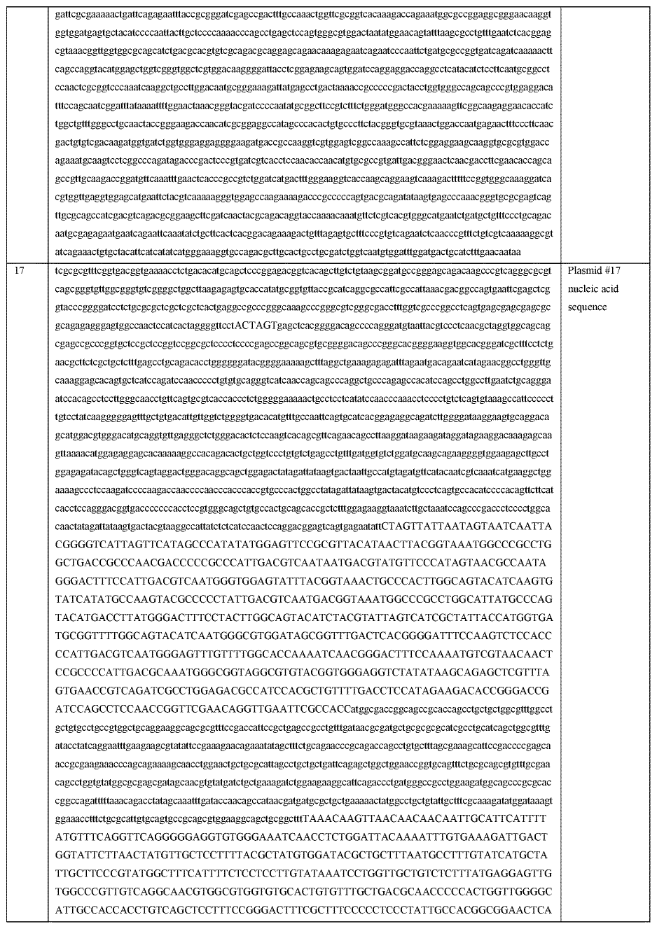

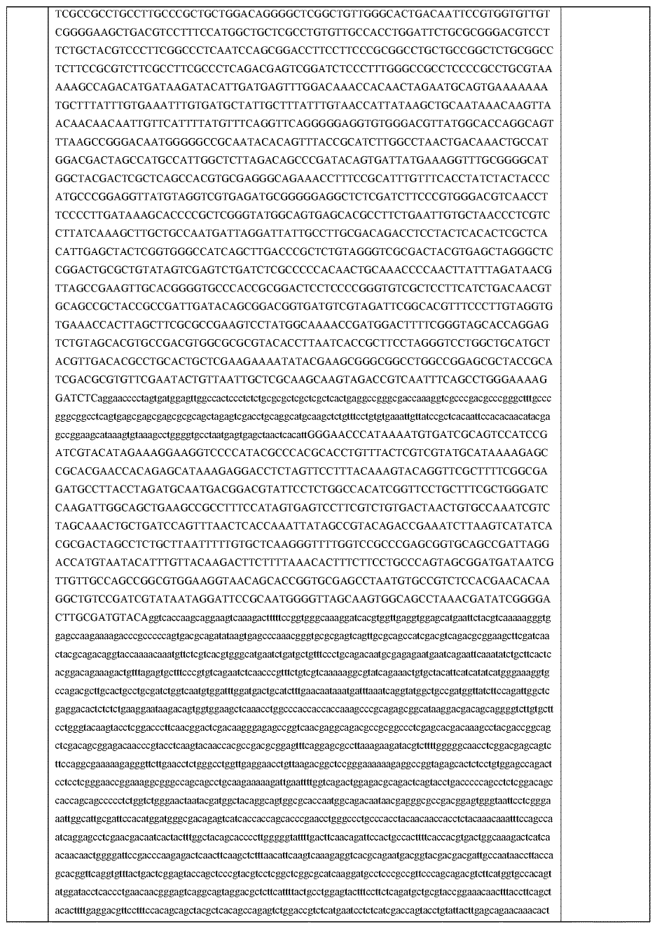

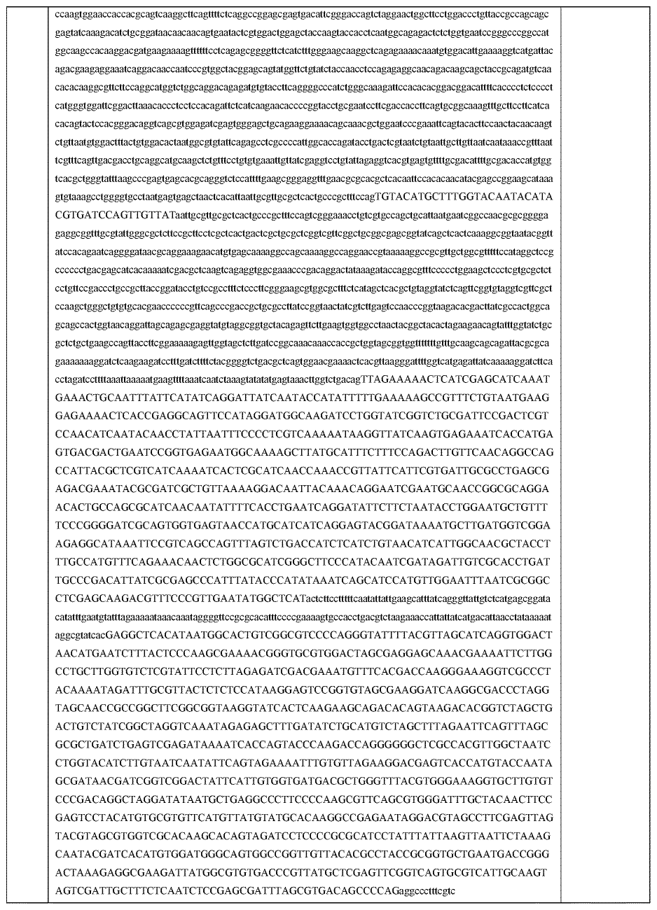

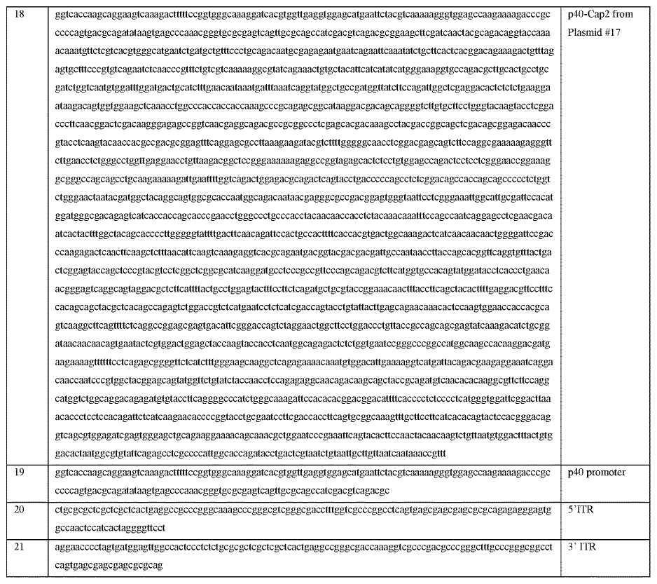

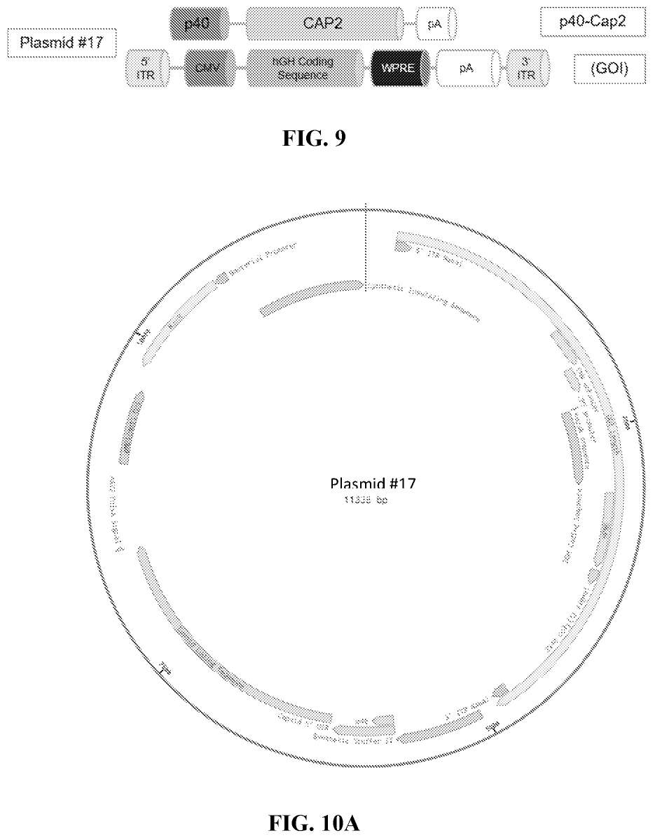

In some cases, the rAAV vectors comprise a) a sequence encoding an AAV capsid, wherein isolated nucleic acid molecule does not comprise a sequence encoding AAV rep; and b) an exogenous gene flanked by inverted terminal repeat (ITR) sequences (e.g., Plasmid #17; see e.g., FIG. 9). In some cases, the sequence encoding the AAV capsid comprises a promoter upstream of the sequence encoding the AAV capsid. The promoter can be any strong constitutive promoter. In some cases, the promoter is p40 promoter. The promoter can be operably linked to the capsid coding sequence. The AAV capsid can be any capsid disclosed herein, e.g., AAV1, AAV2, AAV2 variants, AAV3 (including AAV3A and AAV3B), AAV4, AAV5, AAV6, AAV7, AAV8, AAV9, AAV9 variants, AAV10, AAV11, AAV12, AAV13, AAV-Rh10, AAV-Rh74, AAV-2i8, AAV-DJ, LK03, NP59, or KP1, or any other AAVs. In some cases, the AAV capsid is AAV2, AAV6, AAV7, AAV8, or AAV9 capsid. In some cases, the exogenous gene further comprises a promoter, a post-transcriptional regulatory element, or a poly(A) sequence. In some cases, the promoter is a CMV promoter. The promoter can be operably linked to the exogenous gene. In some cases, the post-transcriptional regulatory element is WPRE. The post-transcriptional regulatory element can be operably linked to the exogenous gene. In some cases, the rAAV vectors comprise, as operatively linked components, a first nucleic acid sequence comprising promoter, a capsid coding sequence, and a poly(A) sequence; and a second nucleic acid sequence comprising a 5′ ITR, a promoter, a GOI coding sequence, a WPRE, a poly(A) sequence, and a 3′ ITR. In some cases, the promoter in the first nucleic acid sequence is a p40 promoter. In some cases, the promoter in the second nucleic acid sequence is a CMV promoter.

In some cases, the rAAV vector comprises a nucleic acid sequence with about 70%, about 75%, about 80%, about 85%, about 90%, about 95%, about 96%, about 97%, about 98%, about 99%, about 99.5%, or about 99.9% sequence identity to SEQ ID NO: 17. In some cases, the rAAV vector comprises a nucleic acid sequence with at least 70%, at least 75%, at least 80%, at least 85%, at least 90%, at least 95%, at least 96%, at least 97%, at least 98%, at least 99%, at least 99.5%, or at least 99.9% sequence identity to SEQ ID NO: 17. In some cases, the rAAV vector comprises a sequence of SEQ ID NO: 17. In some cases, the rAAV vector consists of a nucleic acid sequence with about 70%, about 75%, about 80%, about 85%, about 90%, about 95%, about 96%, about 97%, about 98%, about 99%, about 99.5%, or about 99.9% sequence identity to SEQ ID NO: 17. In some cases, the rAAV vector consists of a nucleic acid sequence with at least 70%, at least 75%, at least 80%, at least 85%, at least 90%, at least 95%, at least 96%, at least 97%, at least 98%, at least 99%, at least 99.5%, or at least 99.9% sequence identity to SEQ ID NO: 17. In some cases, the rAAV vector consists of a sequence of SEQ ID NO: 17.

In some cases, the rAAV vectors comprise a) sequence encoding an AAV rep protein or a sequence encoding an AAV capsid, and a sequence encoding a non-AUG translation start codon 5′ of the sequence encoding an AAV rep protein or a sequence encoding an AAV capsid; and b) one or more helper elements (e.g., Plasmid #11, see e.g., FIG. 2A). In some cases, the rAAV vector comprises the sequence encoding the AAV rep protein. In some cases, the non-AUG translation start codon is ACG. The AAV rep protein can be any rep protein disclosed herein, e.g., AAV2 rep protein. The AAV capsid can be any capsid disclosed herein, e.g., AAV1, AAV2, AAV2 variants, AAV3 (including AAV3A and AAV3B), AAV4, AAV5, AAV6, AAV7, AAV8, AAV9, AAV9 variants, AAV10, AAV11, AAV12, AAV13, AAV-Rh10, AAV-Rh74, AAV-2i8, AAV-DJ, LK03, NP59, or KP1, or any other AAVs. In some cases, the AAV capsid is AAV2, AAV6, AAV7, AAV8, or AAV9 capsid. In some cases, the non-AUC start codon is upstream of the sequence encoding an AAV rep protein. The non-AUC start codon can be at the 5′ end of the sequence encoding an AAV rep protein. In some cases, the first nucleic acid sequence comprises a promoter upstream of the sequence encoding the rep protein. The promoter can be operably linked to the 5′ end of the sequence encoding the rep protein. In some cases, the promoter is a p5 promoter. In some cases, the sequence encoding the AAV rep protein or the sequence encoding the AAV capsid comprises a poly(A) sequence. The poly(A) sequence can be operably linked to the 3′ end of the rep protein coding sequence or the 3′ end of the capsid coding sequence. The one or more helper element can be any element having the helper function for rAAV packaging. In some cases, the one or more helper element is VA gene, E2A(DBP) gene, or E4 gene. In some cases, the one or more helper element is from Ad1, Ad2, Ad3, Ad4, Ad5, Ad6, Ad7, Ad8, Ad9, Ad10, Ad11, Ad12, Ad13, Ad14, Ad15, Ad16, Ad17, Ad18, Ad19, Ad20, Ad21, Ad22, Ad23, Ad24, Ad25, Ad26, Ad27, Ad28, Ad29, Ad30, Ad31, Ad32, Ad33, Ad34, Ad35, Ad36, Ad37, Ad38, Ad39, Ad40, Ad41, Ad42, Ad43, Ad44, Ad45, Ad46, Ad47, Ad48, Ad49, Ad50, Ad51 or Ad52. In some cases, the one or more helper element is from Ad1, Ad2, Ad5, or Ad6. In some cases, the one or more helper element is from Ad2 or Ad5. In some cases, the one or more helper element is from Ad5. In some cases, the rAAV vectors comprises, as operatively linked components, a first nucleic acid sequence comprising a p5 promoter, a non-AUG start codon, a rep protein coding sequence, a capsid coding sequence, a poly(A) sequence; and a second nucleic acid sequence comprising VA gene, E2A(DBP) gene, and E4 gene. In some cases, the non-AUG start codon is ACG.

In some cases, the rAAV vector comprises a nucleic acid sequence with about 70%, about 75%, about 80%, about 85%, about 90%, about 95%, about 96%, about 97%, about 98%, about 99%, about 99.5%, or about 99.9% sequence identity to SEQ ID NO: 3. In some cases, the rAAV vector comprises a nucleic acid sequence with at least 70%, at least 75%, at least 80%, at least 85%, at least 90%, at least 95%, at least 96%, at least 97%, at least 98%, at least 99%, at least 99.5%, or at least 99.9% sequence identity to SEQ ID NO: 3. In some cases, the rAAV vector comprises a sequence of SEQ ID NO: 3. In some cases, the rAAV vector consists of a nucleic acid sequence with about 70%, about 75%, about 80%, about 85%, about 90%, about 95%, about 96%, about 97%, about 98%, about 99%, about 99.5%, or about 99.9% sequence identity to SEQ ID NO: 3. In some cases, the rAAV vector consists of a nucleic acid sequence with at least 70%, at least 75%, at least 80%, at least 85%, at least 90%, at least 95%, at least 96%, at least 97%, at least 98%, at least 99%, at least 99.5%, or at least 99.9% sequence identity to SEQ ID NO: 3. In some cases, the rAAV vector consists of a sequence of SEQ ID NO: 3.

In some cases, the rAAV vectors comprise a sequence encoding an AAV rep protein, wherein the isolated nucleic acid molecule does not comprise a sequence encoding a capsid (e.g., Plasmid #12; see e.g., FIG. 2B). The rep protein can be any rep protein disclosed herein, e.g., AAV2 rep protein. In some cases, the rAAV vector comprises one or more helper element. The one or more helper element can be any element having the helper function for rAAV packaging. In some cases, the one or more helper element is VA gene, E2A(DBP) gene, or E4 gene. In some cases, the one or more helper element is from Ad2 or Ad5. In some cases, the rAAV comprises a p5 promoter 5′ of the sequence encoding the AAV rep protein. In some cases, the rAAV vector further comprises a sequence encoding a non-AUG translation start codon 5′ of the sequence encoding the AAV rep protein. In some cases, the non-AUG translation start codon is ACG. In some cases, the rAAV vectors comprises, as operatively linked components, a first nucleic acid sequence comprising a p5 promoter, a non-AUG start codon, a rep protein coding sequence, a poly(A) sequence; and a second nucleic acid sequence comprising VA gene, E2A(DBP) gene, and E4 gene.

In some cases, the rAAV vector comprises a nucleic acid sequence with about 70%, about 75%, about 80%, about 85%, about 90%, about 95%, about 96%, about 97%, about 98%, about 99%, about 99.5%, or about 99.9% sequence identity to SEQ ID NO: 2. In some cases, the rAAV vector comprises a nucleic acid sequence with at least 70%, at least 75%, at least 80%, at least 85%, at least 90%, at least 95%, at least 96%, at least 97%, at least 98%, at least 99%, at least 99.5%, or at least 99.9% sequence identity to SEQ ID NO: 2. In some cases, the rAAV vector comprises a sequence of SEQ ID NO: 2. In some cases, the rAAV vector consists of a nucleic acid sequence with about 70%, about 75%, about 80%, about 85%, about 90%, about 95%, about 96%, about 97%, about 98%, about 99%, about 99.5%, or about 99.9% sequence identity to SEQ ID NO: 2. In some cases, the rAAV vector consists of a nucleic acid sequence with at least 70%, at least 75%, at least 80%, at least 85%, at least 90%, at least 95%, at least 96%, at least 97%, at least 98%, at least 99%, at least 99.5%, or at least 99.9% sequence identity to SEQ ID NO: 2. In some cases, the rAAV vector consists of a sequence of SEQ ID NO: 2.

In some cases, the rAAV vectors comprise a) a sequence encoding an AAV rep protein or a sequence encoding an AAV capsid, wherein a promoter is 3′ downstream of the sequence encoding the AAV rep protein or sequence encoding the AAV capsid; and b) one or more helper element (e.g., Plasmid #13; see e.g., FIG. 2C). The AAV rep protein can be any rep protein disclosed herein, e.g., AAV2 rep protein. The AAV capsid can be any capsid disclosed herein, e.g., AAV1, AAV2, AAV2 variants, AAV3 (including AAV3A and AAV3B), AAV4, AAV5, AAV6, AAV7, AAV8, AAV9, AAV9 variants, AAV10, AAV11, AAV12, AAV13, AAV-Rh10, AAV-Rh74, AAV-2i8, AAV-DJ, LK03, NP59, or KP1, or any other AAVs. In some cases, the AAV capsid is AAV2, AAV6, AAV7, AAV8, or AAV9 capsid. In some cases, the sequence encoding the AAV rep protein or the sequence encoding the AAV capsid comprises a promoter downstream of the sequence encoding the capsid or rep protein. The promoter can be operably linked to the 3′ end of the sequence encoding the capsid. In some cases, the sequence encoding the AAV rep protein or the sequence encoding the AAV capsid comprises a poly(A) sequence at the 3′ end of the sequence encoding the capsid or the rep protein. In some cases, the promoter is operably linked to the 3′ end of the poly(A) sequence. In some cases, the promoter is a p5 promoter. The one or more helper element can be any element having the helper function for rAAV packaging. In some cases, the one or more helper element is VA gene, E2A(DBP) gene, or E4 gene. In some cases, the one or more helper element is from Ad2 or Ad5. In some cases, the rAAV vectors comprises, as operatively linked components, a first nucleic acid sequence comprising an AAV rep protein coding sequence, an AAV capsid coding sequence, a poly(A) sequence, a p5 promoter; and a second nucleic acid sequence comprising VA gene, E2A(DBP) gene, and E4 gene.

In some cases, the rAAV vectors comprise a) a sequence encoding an AAV rep protein or a sequence encoding an AAV capsid, wherein a p5 promoter is not 5′ of the sequence encoding the AAV rep protein or sequence encoding the AAV capsid; and b) one or more helper elements (see e.g., Plasmid #13; see e.g., FIG. 2C). In some cases, the rAAV vector comprises the sequence encoding the AAV rep protein. The AAV rep protein can be any rep protein disclosed herein, e.g., AAV2 rep protein. In some cases, the rAAV vector comprises the sequence encoding the AAV capsid. The AAV capsid can be any capsid disclosed herein, e.g., AAV1, AAV2, AAV2 variants, AAV3 (including AAV3A and AAV3B), AAV4, AAV5, AAV6, AAV7, AAV8, AAV9, AAV9 variants, AAV10, AAV11, AAV12, AAV13, AAV-Rh10, AAV-Rh74, AAV-2i8, AAV-DJ, LK03, NP59, or KP1, or any other AAVs. In some cases, the AAV capsid is AAV2, AAV6, AAV7, AAV8, or AAV9 capsid. In some cases, the one or more helper element is VA gene, E2A(DBP) gene, or E4 gene. In some cases, the one or more helper element is from Ad2 or Ad5. In some cases, the rAAV vector comprises the sequence encoding the AAV rep protein and the AAV capsid. In some cases, the rAAV vector further comprises a promoter 3′ of the sequence encoding the AAV rep protein and the sequence encoding the AAV capsid.

In some cases, the rAAV vector comprises a nucleic acid sequence with about 70%, about 75%, about 80%, about 85%, about 90%, about 95%, about 96%, about 97%, about 98%, about 99%, about 99.5%, or about 99.9% sequence identity to SEQ ID NO: 1. In some cases, the rAAV vector comprises a nucleic acid sequence with at least 70%, at least 75%, at least 80%, at least 85%, at least 90%, at least 95%, at least 96%, at least 97%, at least 98%, at least 99%, at least 99.5%, or at least 99.9% sequence identity to SEQ ID NO: 1. In some cases, the rAAV vector comprises a sequence of SEQ ID NO: 1. In some cases, the rAAV vector consists of a nucleic acid sequence with about 70%, about 75%, about 80%, about 85%, about 90%, about 95%, about 96%, about 97%, about 98%, about 99%, about 99.5%, or about 99.9% sequence identity to SEQ ID NO: 1. In some cases, the rAAV vector consists of a nucleic acid sequence with at least 70%, at least 75%, at least 80%, at least 85%, at least 90%, at least 95%, at least 96%, at least 97%, at least 98%, at least 99%, at least 99.5%, or at least 99.9% sequence identity to SEQ ID NO: 1. In some cases, the rAAV vector consists of a sequence of SEQ ID NO: 1.

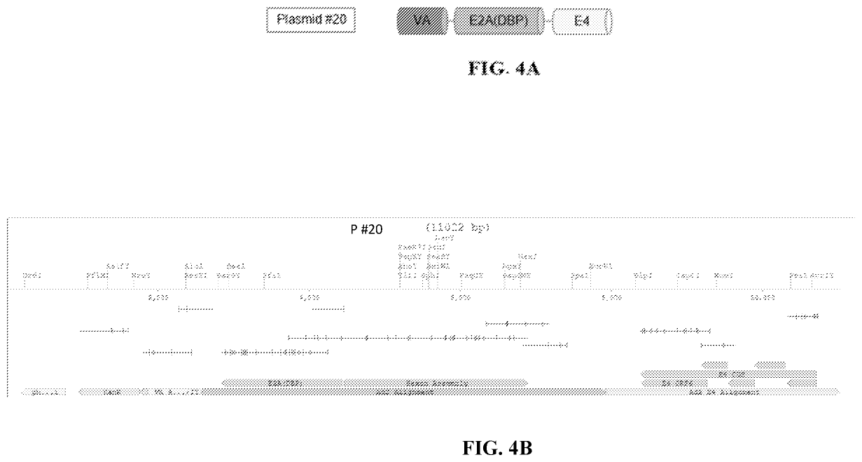

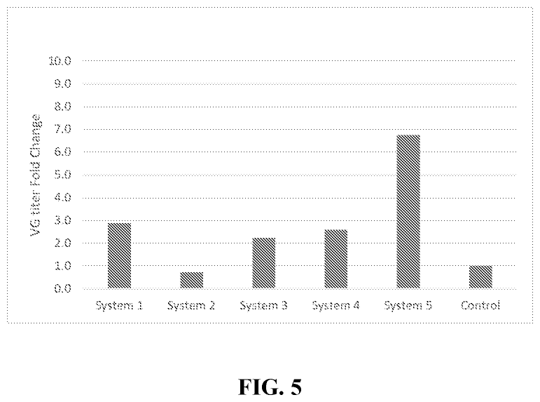

In some cases, the rAAV vector comprises one or more helper elements from an adenovirus, wherein i) the rAAV vector does not comprise an Ad5 Fiber sequence, or ii) the rAAV vector is less than 16.5 kilobases (e.g., Plasmid #20, FIG. 4A). In some cases, the rAAV vector is less than 16 kilobases, less than 15.5 kilobases, less than 15 kilobases, less than 14.5 kilobases, less than 14 kilobases, less than 13.5 kilobases, less than 13 kilobases, less than 12.5 kilobases, less than 12 kilobases, less than 11.9 kilobases, less than 11.8 kilobases, less than 11.7 kilobases, less than 11.6 kilobases, less than 11.5 kilobases, less than 11.4 kilobases less than 11.3 kilobases, less than 11.2 kilobases, less than 11.1 kilobases, less than 11 kilobases, less than 10.9 kilobases, less than 10.8 kilobases, less than 10.7 kilobases, less than 10.6 kilobases, less than 10.5 kilobases, less than 10.4 kilobases, less than 10.3 kilobases, less than 10.2 kilobases, less than 10.1 kilobases, or less than 10 kilobases. In some cases, the rAAV vector is less than 16.5 kilobases. In some cases, the rAAV vector is less than 15.5 kilobases. In some cases, the rAAV vector is less than 14.5 kilobases. In some cases, the rAAV vector is less than 13.5 kilobases. In some cases, the rAAV vector is less than 12.5 kilobases. In some cases, the rAAV vector is less than 11.5 kilobases. In some cases, the rAAV vector is less than 11.3 kilobases. In some cases, the rAAV vector is from about 16.5 to about 7.5 kilobases. In some cases, the rAAV vector is from about 16 to about 8 kilobases. In some cases, the rAAV vector is from about 15.5 to about 8.5 kilobases. In some cases, the rAAV vector is from about 15 to about 9 kilobases. In some cases, the rAAV vector is from about 14.5 to about 9.5 kilobases. In some cases, the rAAV vector is from about 14 to about 10 kilobases. In some cases, the rAAV vector is from about 13.5 to about 10.5 kilobases. In some cases, the rAAV vector is from about 13 to about 11 kilobases. In some cases, the rAAV vector is from about 12.5 to about 11.5 kilobases. In some cases, the rAAV vector is about 11.5 kilobases. In some cases, the rAAV vector is about 11.4 kilobases. In some cases, the rAAV vector is about 11.3 kilobases. In some cases, the rAAV vector is about 11.2 kilobases. In some cases, the rAAV vector is about 11.1 kilobases. In some cases, the rAAV vector is about 11 kilobases. In some cases, the adenovirus is Adenovirus 5 (Ad5) or Adenovirus 2 (Ad2). In some cases, each of the one or more helper elements is from the same adenovirus. In some cases, the one or more helper elements are from different adenovirus. In some cases, the one or more helper elements comprises Ad5-VA, Ad5-E2A(DBP), or Ad5-E4. In some cases, the one or more helper elements comprises Ad2-VA, Ad2-E2A(DBP), or Ad2-E4. In some cases, the rAAV vector does not comprise an Ad5 Fiber sequence. In some cases, the rAAV vector is less than 16.5 kilobases. In some cases, the rAAV vector does not comprise an Ad5 Fiber sequence, and the rAAV vector is less than 16.5 kilobases. In some cases, the rAAV vector does not comprise an Ad5 Fiber sequence, and the rAAV vector is less than 16 kilobases, less than 15.5 kilobases, less than 15 kilobases, less than 14.5 kilobases, less than 14 kilobases, less than 13.5 kilobases, less than 13 kilobases, less than 12.5 kilobases, less than 12 kilobases, less than 11.9 kilobases, less than 11.8 kilobases, less than 11.7 kilobases, less than 11.6 kilobases, less than 11.5 kilobases, less than 11.4 kilobases less than 11.3 kilobases, less than 11.2 kilobases, less than 11.1 kilobases, less than 11 kilobases, less than 10.9 kilobases, less than 10.8 kilobases, less than 10.7 kilobases, less than 10.6 kilobases, less than 10.5 kilobases, less than 10.4 kilobases, less than 10.3 kilobases, less than 10.2 kilobases, less than 10.1 kilobases, or less than 10 kilobases. In some cases, the rAAV vector does not comprise an Ad5 Fiber sequence, and the rAAV vector is less than 16.5 kilobases. In some cases, the rAAV vector does not comprise an Ad5 Fiber sequence, and the rAAV vector is less than 15.5 kilobases. In some cases, the rAAV vector does not comprise an Ad5 Fiber sequence, and the rAAV vector is less than 14.5 kilobases. In some cases, the rAAV vector does not comprise an Ad5 Fiber sequence, and the rAAV vector is less than 13.5 kilobases. In some cases, the rAAV vector does not comprise an Ad5 Fiber sequence, and the rAAV vector is less than 12.5 kilobases. In some cases, the rAAV vector does not comprise an Ad5 Fiber sequence, and the rAAV vector is less than 11.5 kilobases. In some cases, the rAAV vector does not comprise an Ad5 Fiber sequence, and the rAAV vector is less than 11.3 kilobases. In some cases, the rAAV vector does not comprise an Ad5 Fiber sequence, and the rAAV vector is from about 16.5 to about 7.5 kilobases. In some cases, the rAAV vector does not comprise an Ad5 Fiber sequence, and the rAAV vector is from about 16 to about 8 kilobases. In some cases, the rAAV vector does not comprise an Ad5 Fiber sequence, and the rAAV vector is from about 15.5 to about 8.5 kilobases. In some cases, the rAAV vector does not comprise an Ad5 Fiber sequence, and the rAAV vector is from about 15 to about 9 kilobases. In some cases, the rAAV vector does not comprise an Ad5 Fiber sequence, and the rAAV vector is from about 14.5 to about 9.5 kilobases. In some cases, the rAAV vector does not comprise an Ad5 Fiber sequence, and the rAAV vector is from about 14 to about 10 kilobases. In some cases, the rAAV vector does not comprise an Ad5 Fiber sequence, and the rAAV vector is from about 13.5 to about 10.5 kilobases. In some cases, the rAAV vector does not comprise an Ad5 Fiber sequence, and the rAAV vector is from about 13 to about 11 kilobases. In some cases, the rAAV vector does not comprise an Ad5 Fiber sequence, and the rAAV vector is from about 12.5 to about 11.5 kilobases. In some cases, the rAAV vector does not comprise an Ad5 Fiber sequence, and the rAAV vector is about 11.5 kilobases. In some cases, the rAAV vector does not comprise an Ad5 Fiber sequence, and the rAAV vector is about 11.4 kilobases. In some cases, the rAAV vector does not comprise an Ad5 Fiber sequence, and the rAAV vector is about 11.3 kilobases. In some cases, the rAAV vector does not comprise an Ad5 Fiber sequence, and the rAAV vector is about 11.2 kilobases. In some cases, the rAAV vector does not comprise an Ad5 Fiber sequence, and the rAAV vector is about 11.1 kilobases. In some cases, the rAAV vector does not comprise an Ad5 Fiber sequence, and the rAAV vector is about 11 kilobases.

In some cases, the rAAV vector comprises a nucleic acid sequence with about 70%, about 75%, about 80%, about 85%, about 90%, about 95%, about 96%, about 97%, about 98%, about 99%, about 99.5%, or about 99.9% sequence identity to SEQ ID NO: 7. In some cases, the rAAV vector comprises a nucleic acid sequence with at least 70%, at least 75%, at least 80%, at least 85%, at least 90%, at least 95%, at least 96%, at least 97%, at least 98%, at least 99%, at least 99.5%, or at least 99.9% sequence identity to SEQ ID NO: 7. In some cases, the rAAV vector comprises a sequence of SEQ ID NO: 7.

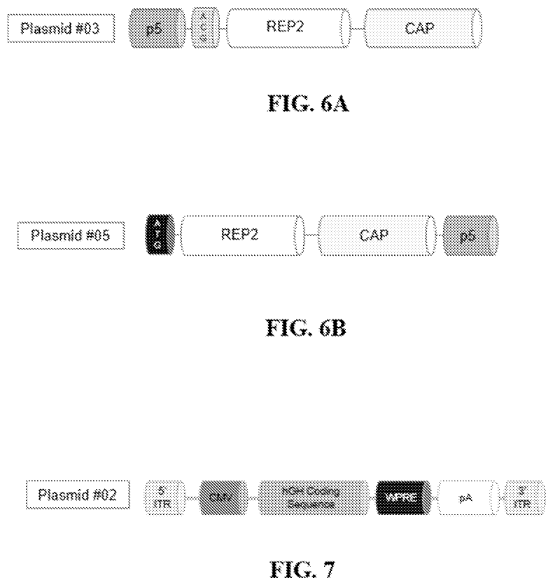

In some cases, the rAAV vector comprises a nucleic acid sequence encoding an AAV rep protein and/or an AAV capsid (e.g., Plasmid #03, FIG. 6A). In some cases, the rAAV vector comprises a nucleic acid sequence with about 70%, about 75%, about 80%, about 85%, about 90%, about 95%, about 96%, about 97%, about 98%, about 99%, about 99.5%, or about 99.9% sequence identity to SEQ ID NO: 11. In some cases, the rAAV vector comprises a nucleic acid sequence with at least 70%, at least 75%, at least 80%, at least 85%, at least 90%, at least 95%, at least 96%, at least 97%, at least 98%, at least 99%, at least 99.5%, or at least 99.9% sequence identity to SEQ ID NO: 11. In some cases, the rAAV vector comprises a sequence of SEQ ID NO: 11.

In some cases, the rAAV vector comprises a promoter downstream of a sequence encoding an AAV rep protein or sequence encoding an AAV capsid and lacking a p5 promoter upstream of the sequence encoding the AAV rep protein or sequence encoding the AAV capsid (e.g., Plasmid #05, FIG. 6B). In some cases, the rAAV vector comprises the sequence encoding the AAV rep protein and the sequence encoding the AAV capsid. The AAV rep protein can be any rep protein disclosed herein, e.g., AAV2 rep protein. The AAV capsid can be any capsid disclosed herein, e.g., AAV2, AAV6, AAV7, AAV8, or AAV9 capsid. In some cases, the promoter downstream of the sequence encoding the AAV rep protein or sequence encoding the AAV capsid comprises a p5 promoter.

In some cases, the rAAV vector comprises a nucleic acid sequence with about 70%, about 75%, about 80%, about 85%, about 90%, about 95%, about 96%, about 97%, about 98%, about 99%, about 99.5%, or about 99.9% sequence identity to SEQ ID NO: 12. In some cases, the rAAV vector comprises a nucleic acid sequence with at least 70%, at least 75%, at least 80%, at least 85%, at least 90%, at least 95%, at least 96%, at least 97%, at least 98%, at least 99%, at least 99.5%, or at least 99.9% sequence identity to SEQ ID NO: 12. In some cases, the rAAV vector comprises a sequence of SEQ ID NO: 12.

In some cases, the rAAV vector comprises a nucleic acid sequence encoding an AAV rep protein without a sequence encoding an AAV capsid. In some cases, the rAAV vector comprises a nucleic acid sequence with about 70%, about 75%, about 80%, about 85%, about 90%, about 95%, about 96%, about 97%, about 98%, about 99%, about 99.5%, or about 99.9% sequence identity to SEQ ID NO: 13. In some cases, the rAAV vector comprises a nucleic acid sequence with at least 70%, at least 75%, at least 80%, at least 85%, at least 90%, at least 95%, at least 96%, at least 97%, at least 98%, at least 99%, at least 99.5%, or at least 99.9% sequence identity to SEQ ID NO13. In some cases, the rAAV vector comprises a sequence of SEQ ID NO: 13.

In some cases, the rAAV vector comprises a nucleic acid sequence encoding an AAV capsid without a sequence encoding an AAV rep protein. In some cases, the rAAV vector comprises a nucleic acid sequence with about 70%, about 75%, about 80%, about 85%, about 90%, about 95%, about 96%, about 97%, about 98%, about 99%, about 99.5%, or about 99.9% sequence identity to SEQ ID NO: 14 or 18. In some cases, the rAAV vector comprises a nucleic acid sequence with at least 70%, at least 75%, at least 80%, at least 85%, at least 90%, at least 95%, at least 96%, at least 97%, at least 98%, at least 99%, at least 99.5%, or at least 99.9% sequence identity to SEQ ID NO: 14 or 18. In some cases, the rAAV vector comprises a sequence of SEQ ID NO: 14 or 18.

In some cases, the rAAV vector comprises an exogenous gene. The exogenous gene can be flanked by ITRs at 5′ and 3′. In some cases, the rAAV vector lacks a nucleic acid sequence encoding an AAV rep protein. In some cases, the rAAV vector lacks a nucleic acid sequence encoding an AAV capsid. In some cases, the rAAV vector lacks nucleic acid sequences encoding AAV capsid and rep protein. In some cases, the rAAV vector lacks a helper element. In some cases, the rAAV lacks a nucleic acid sequence encoding an AAV rep protein and a helper element. In some cases, the rAAV lacks a nucleic acid sequence encoding an AAV capsid and a helper element. In some cases, the rAAV lacks (i) a nucleic acid sequence encoding an AAV capsid, (ii) a nucleic acid sequence encoding an AAV rep protein, and (iii) a helper element. In some cases, the exogenous gene is hGH (e.g., Plasmid #02, FIG. 7). In some cases, the rAAV vector comprises a nucleic acid sequence with about 70%, about 75%, about 80%, about 85%, about 90%, about 95%, about 96%, about 97%, about 98%, about 99%, about 99.5%, or about 99.9% sequence identity to SEQ ID NO: 6. In some cases, the rAAV vector comprises a nucleic acid sequence with at least 70%, at least 75%, at least 80%, at least 85%, at least 90%, at least 95%, at least 96%, at least 97%, at least 98%, at least 99%, at least 99.5%, or at least 99.9% sequence identity to SEQ ID NO: 6. In some cases, the rAAV vector comprises a sequence of SEQ ID NO: 6.

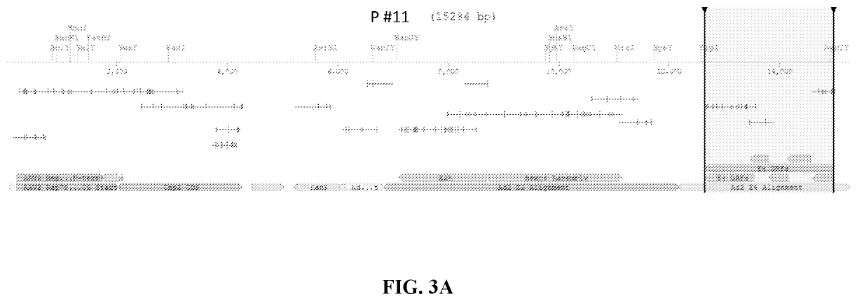

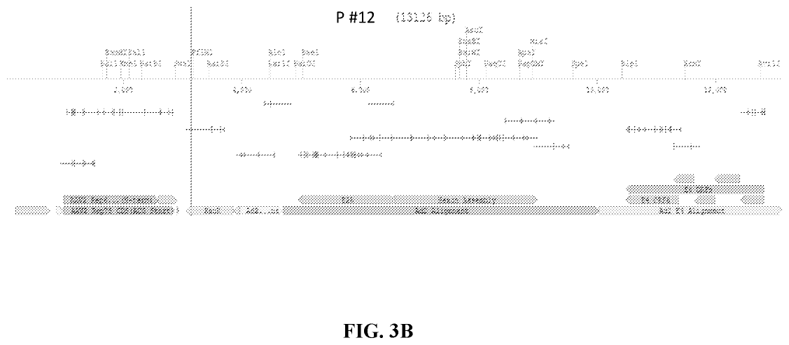

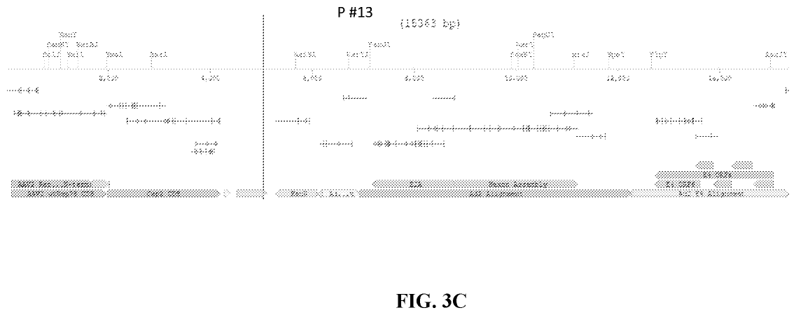

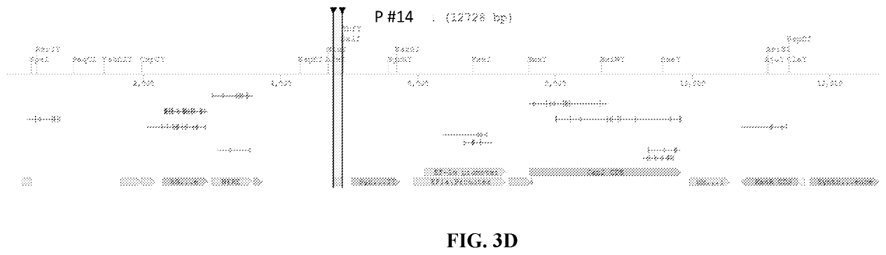

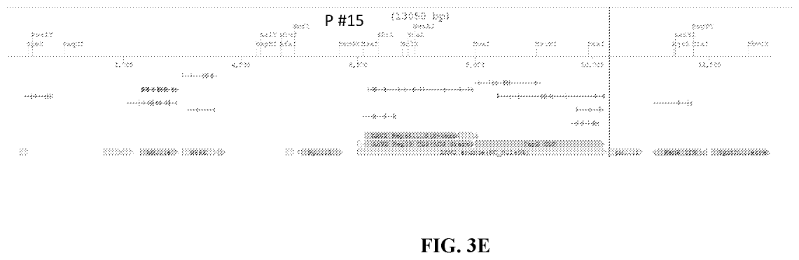

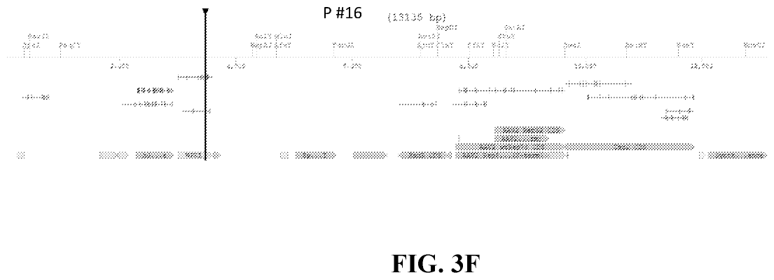

In some cases, the rAAV vector comprises a plasmid map shown in FIG. 3A. In some cases, the rAAV vector comprises a plasmid map shown in FIG. 3B. In some cases, the rAAV vector comprises a plasmid map shown in FIG. 3C. In some cases, the rAAV vector comprises a plasmid map shown in FIG. 3D. In some cases, the rAAV vector comprises a plasmid map shown in FIG. 3E. In some cases, the rAAV vector comprises a plasmid map shown in FIG. 3F.

### Helper Elements

Provided herein include nucleic acid sequence comprising one or more helper elements. The one or more helper elements comprise nucleotide sequences encoding proteins that provide helper functions. The one or more helper elements can be from helper viruses such as Adenoviruses (Ad), herpes simplex virus (HSV), or human papillomavirus (HPV). In some cases, the one or more helper elements can be from different serotypes of adenovirus (Ad), including, but not are limited, Ad2 and Ad5. In some cases, the nucleic acid sequence comprises one helper elements. In some cases, the nucleic acid sequence comprises two helper elements. In some cases, the nucleic acid sequence comprises three helper elements. In some cases, the nucleic acid sequence comprises four helper elements. In some cases, the nucleic acid sequence comprises five helper elements. In some cases, the nucleic acid sequence comprises at least two helper elements. In some cases, the nucleic acid sequence comprises at least three helper elements. In some cases, the nucleic acid sequence comprises at least four helper elements. In some cases, the nucleic acid sequence comprises at least five helper elements. In some cases, the nucleic acid sequence comprising the one or more helper elements includes E1A, E1B, VA, E2A DNA-binding protein (DBP) or E4 region from an adenovirus. In some cases, the helper element is E1A, E1B, VA, E2A DNA-binding protein (DBP) or E4 region from an adenovirus. In some cases, the nucleic acid sequence comprising the one or more helper elements includes E4, VA, and E2A(DBP) regions from an adenovirus. In some cases, the nucleic acid sequence comprising one or more helper element does not include a nucleic acid sequence that are dispensable in AAV production, for example, Fiber and Hexon Assembly from adenovirus. In some cases, the nucleic acid sequence comprising one or more helper element lacks Fiber or Hexon Assembly from adenovirus. In some cases, the nucleic acid sequence comprising one or more helper element lacks both Fiber and Hexon Assembly from adenovirus.

In some cases, the nucleic acid sequence comprising one or more helper element has at least about 70%, about 75%, about 80%, about 85%, about 90%, about 95%, about 99% sequence identity to SEQ ID NO: 8. In some cases, the nucleic acid sequence comprising one or more helper element comprises the sequence of SEQ ID NO: 8. In some cases, the nucleic acid sequence comprising one or more helper element has at least about 70%, about 75%, about 80%, about 85%, about 90%, about 95%, about 99% sequence identity to SEQ ID NO: 9. In some cases, the nucleic acid sequence comprising one or more helper element comprises the sequence of SEQ ID NO: 9. In some cases, the nucleic acid sequence comprising one or more helper element has at least about 70%, about 75%, about 80%, about 85%, about 90%, about 95%, about 99% sequence identity to SEQ ID NO: 10. In some cases, the nucleic acid sequence comprising one or more helper element comprises the sequence of SEQ ID NO: 10.

Also disclosed herein are vectors comprising the nucleic acid sequence comprising one or more helper element. In some cases, the vector is a plasmid. The vector comprising one or more helper element disclosed herein can have a small size which allows efficient transfection.

In some cases, the vector has a size of less than 21 kbp, less than 20.5 kbp, less than 20 kbp, less than 19.5 kbp, less than 19 kbp, less than 18.5 kbp, less than 18 kbp, less than 17.5 kbp, less than 17 kbp, less than 16.5 kbp, less than 16 kbp, less than 15.5 kbp, less than 15 kbp, less than 14.5 kbp, less than 14 kbp, less than 13.5 kbp, less than 13 kbp, less than 12.5 kbp, less than 12 kbp, less than 11.5 kbp, less than 11 kbp, less than 10.5 kbp, or less than 10 kbp. In some cases, the vector has a size of less than 16 kilobases, less than 15.5 kilobases, less than 15 kilobases, less than 14.5 kilobases, less than 14 kilobases, less than 13.5 kilobases, less than 13 kilobases, less than 12.5 kilobases, less than 12 kilobases, less than 11.9 kilobases, less than 11.8 kilobases, less than 11.7 kilobases, less than 11.6 kilobases, less than 11.5 kilobases, less than 11.4 kilobases less than 11.3 kilobases, less than 11.2 kilobases, less than 11.1 kilobases, less than 11 kilobases, less than 10.9 kilobases, less than 10.8 kilobases, less than 10.7 kilobases, less than 10.6 kilobases, less than 10.5 kilobases, less than 10.4 kilobases, less than 10.3 kilobases, less than 10.2 kilobases, less than 10.1 kilobases, or less than 10 kilobases. In some cases, the vector has a size less than 16.5 kilobases. In some cases, the vector has a size less than 15.5 kilobases. In some cases, the vector has a size less than 14.5 kilobases. In some cases, the vector has a size less than 13.5 kilobases. In some cases, the vector has a size less than 12.5 kilobases. In some cases, the vector has a size less than 11.5 kilobases. In some cases, the vector has a size less than 11.3 kilobases.

In some cases, the vector has a size from about 22 kbp to about 11 kbp, from about 21.5 kbp to about 11.5 kbp, from about 21 kbp to about 12 kbp, from about 20.5 kbp to about 12.5 kbp, from about 20 kbp to about 13 kbp, from about 19.5 kbp to about 13.5 kbp, from about 19 kbp to about 14 kbp, from about 18.5 kbp to about 14.5 kbp, from about 18 kbp to about 15 kbp, from about 17.5 kbp to about 15 kbp, from about 17 kbp to about 15.5 kbp, or from about 16.5 kbp to about 16 kbp. In some cases, the vector has a size from about 9 kbp to about 13 kbp, from about 9.5 kbp to about 12.5 kbp, or from about 10 kbp to about 12 kbp. In some cases, the vector has a size from about 16.5 to about 7.5 kilobases. In some cases, the vector has a size from about 16 to about 8 kilobases. In some cases, the vector has a size from about 15.5 to about 8.5 kilobases. In some cases, the vector has a size from about 15 to about 9 kilobases. In some cases, the vector has a size from about 14.5 to about 9.5 kilobases. In some cases, the vector has a size from about 14 to about 10 kilobases. In some cases, the vector has a size from about 13.5 to about 10.5 kilobases. In some cases, the vector has a size from about 13 to about 11 kilobases. In some cases, the vector has a size from about 12.5 to about 11.5 kilobases.

In some cases, the vector has a size of about 11 kbp, about 11.5 kbp, about 12 kbp, about 12.5 kbp, about 13 kbp, about 13.5 kbp, about 14 kbp, about 14.5 kbp, about 15 kbp, about 15.5 kbp, about 16 kbp, about 16.5 kbp, about 17 kbp, about 17.5 kbp, about 18 kbp, about 18.5 kbp, about 19 kbp, about 19.5 kbp, or about 20 kbp. In some cases, the vector has a size of about 16.5 kbp. In some cases, the vector has a size of about 11.5 kilobases. In some cases, the vector has a size of about 11.4 kilobases. In some cases, the vector has a size of about 11.3 kilobases. In some cases, the vector has a size of about 11.2 kilobases. In some cases, the vector has a size of about 11.1 kilobases. In some cases, the vector has a size of about 11 kilobases.

In some cases, the vector comprises a nucleic acid sequence having at least about 70%, about 75%, about 80%, about 85%, about 90%, about 95%, about 99% of SEQ ID NO: 8. In some cases, the vector comprises a sequence of SEQ ID NO: 8. In some cases, the vector comprises a nucleic acid sequence having at least about 70%, about 75%, about 80%, about 85%, about 90%, about 95%, about 99% of SEQ ID NO: 9. In some cases, the vector comprises a sequence of SEQ ID NO: 9. In some cases, the vector comprises a nucleic acid sequence having at least about 70%, about 75%, about 80%, about 85%, about 90%, about 95%, about 99% of SEQ ID NO: 10. In some cases, the vector comprises a sequence of SEQ ID NO: 10. In some cases, the vector comprises a nucleic acid sequence having at least about 70%, about 75%, about 80%, about 85%, about 90%, about 95%, about 99% of SEQ ID NO: 7. In some cases, the vector comprises a sequence of SEQ ID NO: 7. In some cases, the vector consists of a sequence of SEQ ID NO: 7. In some cases, the vector comprising the structure of FIG. 4.

### Rep Protein

Disclosed herein are isolated nucleic acid sequences encoding one or more rep proteins. The nucleic acid sequences can comprise a rep gene region from an AAV. For example, the rep gene region can be from AAV serotype 2 (AAV2). The one or more rep proteins can be Rep78, Rep68, Rep52 or Rep40. The isolated nucleic acid sequences encoding one or more rep proteins can further comprise one or more transcriptional and posttranscriptional regulatory elements, including one or more promoters (e.g., p5) or an artificial translation start site (e.g., a non-AUG start codon, e.g., ACG). In some cases, the rep protein can be any rAAV vector used for replication and packaging of rAAV viral particles. The rep protein can comprise rep 78, rep 68, rep 52 and rep 40. In some cases, the rep protein needs not to include all of rep 78, rep 68, rep 52, and rep 40, as long as it can allow the rAAV vector to replicate and package into rAAV virus particles. In some cases, the rep protein comprises any three of rep 78, rep 68, rep 52 and rep 40. In some cases, the rep protein comprises any two of rep 78, rep 68, rep 52 and rep 40. In some cases, the rep protein comprises any one of rep 78, rep 68, rep 52 and rep 40. In some cases, the rep protein comprises rep 78 and rep 52. In some cases, the rep protein comprises rep 78 and rep 40. In some cases, the rep protein comprises rep 68 and rep 52. In some cases, the rep protein includes rep 68 and rep 40.

The rep 78, rep 68, rep 52 and the rep 40 can be from any AAV serotype. In some cases, the rep 78 can be from AAV serotype 1 (AAV1), AAV serotype 2 (AAV2), AAV2 variants, AAV serotype 3 (AAV3, including serotypes 3A and 3B), AAV serotype 4 (AAV4), AAV serotype 5 (AAV5), AAV serotype 6 (AAV6), AAV serotype 7 (AAV7), AAV serotype 8 (AAV8), AAV serotype 9 (AAV9), AAV9 variants, AAV serotype 10 (AAV10), AAV serotype 11 (AAV11), AAV serotype 12 (AAV12), AAV serotype 13 (AAV13), AAV-Rh10, AAV-Rh74, AAV-2i8, AAV-DJ, LK03, NP59, or KP1, or any other AAV. In some cases, the rep 78 can be from AAV2 or AAV5. In some cases, the rep 78 can be from AAV2. In some cases, the rep 78 is from AAV1, AAV2, AAV2 variants, AAV3, (including AAV 3A and 3B), AAV4, AAV5, AAV6, AAV7, AAV8, AAV9, AAV9 variants, AAV10, AAV11, AAV12, AAV13, AAV-Rh10, AAV-Rh74, AAV-2i8, AAV-DJ, LK03, NP59, or KP1, with at least about 75%, about 80%, about 85%, about 90%, about 95% or about 99% sequence identity to a wild type rep 78 of AAV1, AAV2, AAV2 variants, AAV3, (including AAV 3A and 3B), AAV4, AAV5, AAV6, AAV7, AAV8, AAV9, AAV9 variants, AAV10, AAV11, AAV12, AAV13, AAV-Rh10, AAV-Rh74, AAV-2i8, AAV-DJ, LK03, NP59, or KP1, respectively. In some cases, the rep 78 is from AAV2 or AAV5 with at least about 75%, about 80%, about 85%, about 90%, about 95% or about 99% sequence identity to a wild type rep 78 from AAV2 or AAV5, respectively. In some cases, the rep 78 is from AAV2 or AAV5 with at least about 75%, about 80%, about 85%, about 90%, about 95% or about 99% sequence identity to a wild type rep 78 from AAV2. In some cases, the rep 78 comprises one or more substituted amino acids, deletions and/or additions as compared a wildtype rep 78 from AAV1, AAV2, AAV2 variants, AAV3 (including AAV 3A and 3B), AAV4, AAV5, AAV6, AAV7, AAV8, AAV9, AAV9 variants, AAV10, AAV11, AAV12, AAV13, AAV-Rh10, AAV-Rh74, AAV-2i8, AAV-DJ, LK03, NP59, or KP1. In some cases, the rep 78 comprises one or more amino acid substitutions, deletions and/or additions as compared to a wild type rep 78 from AAV2 or AAV5. In some cases, the rep 78 comprises one or more amino acid substitutions, deletions and/or additions as compared to a wild type rep 78 from AAV2.

In some cases, the rep 68 is from AAV1, AAV2, AAV2 variants, AAV3 (including AAV 3A and 3B), AAV4, AAV5, AAV6, AAV7, AAV8, AAV9, AAV9 variants, AAV10, AAV11, AAV12, AAV13, AAV-Rh10, AAV-Rh74, AAV-2i8, AAV-DJ, LK03, NP59, or KP1, or any other AAV. In some cases, the rep 68 is from AAV2 or AAV5. In some cases, the rep 68 is from AAV2. In some cases, the rep 68 is from AAV1, AAV2, AAV2 variants, AAV3 (including AAV 3A and 3B), AAV4, AAV5, AAV6, AAV7, AAV8, AAV9, AAV9 variants, AAV10, AAV11, AAV12, AAV13, AAV-Rh10, AAV-Rh74, AAV-2i8, AAV-DJ, LK03, NP59, or KP1 with at least about 75%, about 80%, about 85%, about 90%, about 95% or about 99% sequence identity to a wild type rep 68 from AAV1, AAV2, AAV2 variants, AAV3 (including AAV 3A and 3B), AAV4, AAV5, AAV6, AAV7, AAV8, AAV9, AAV9 variants, AAV10, AAV11, AAV12, AAV13, AAV-Rh10, AAV-Rh74, AAV-2i8, AAV-DJ, LK03, NP59, or KP1, respectively. In some embodiments, the rep 68 is from AAV2 or AAV5 with at least about 75%, about 80%, about 85%, about 90%, about 95% or about 99% sequence identity to a wild type rep 68 from AAV2 or AAV5, respectively. In some embodiments, the rep 68 is from AAV2 or AAV5 with at least about 75%, about 80%, about 85%, about 90%, about 95% or about 99% sequence identity to a wild type rep 68 from AAV2. In some cases, the rep 68 has one or more amino acid substitutions, deletions and/or additions as compared to a wild-type rep 68 from AAV1, AAV2, AAV2 variants, AAV3, (including AAV3A and 3B), AAV4, AAV5, AAV6, AAV7, AAV8, AAV9, AAV9 variants, AAV10, AAV11, AAV12, AAV13, AAV-Rh10, AAV-Rh74, AAV-2i8, AAV-DJ, LK03, NP59, or KP1. In some cases, the rep 68 has a one or more amino acid substitutions, deletions and/or additions as compared to a wild type rep 68 from AAV2 or the AAV5. In some cases, the rep 68 has a one or more amino acid substitutions, deletions and/or additions as compared to a wild type rep 68 from AAV2.

In some cases, the rep 52 is from AAV1, AAV2, AAV2 variants, AAV3 (including AAV 3A and 3B), AAV4, AAV5, AAV6, AAV7, AAV8, AAV9, AAV9 variants, AAV10, AAV11, AAV12, AAV13, AAV-Rh10, AAV-Rh74, AAV-2i8, AAV-DJ, LK03, NP59, or KP1, or any other AAV. In some cases, the rep 52 is from AAV2 or AAV5. In some cases, the rep 52 is from AAV2. In some cases, the rep 52 is from AAV1, AAV2, AAV2 variants, AAV3 (including AAV 3A and 3B), AAV4, AAV5, AAV6, AAV7, AAV8, AAV9, AAV9 variants, AAV10, AAV11, AAV12, AAV13, AAV-Rh10, AAV-Rh74, AAV-2i8, AAV-DJ, LK03, NP59, or KP1 with at least about 75%, about 80%, about 85%, about 90%, about 95% or 99% sequence identity to a wild type rep 52 from AAV1, AAV2, AAV2 variants, AAV3 (including AAV 3A and 3B), AAV4, AAV5, AAV6, AAV7, AAV8, AAV9, AAV9 variants, AAV10, AAV11, AAV12, AAV13, AAV-Rh10, AAV-Rh74 or AAV-2i8, AAV-DJ, LK03, NP59, or KP1, respectively. In some cases, the rep 52 is from AAV2 or the AAV5 with at least about 75%, about 80%, about 85%, about 90%, about 95% or about 99% sequence identity to a wild type rep 52 from AAV2 or AAV5, respectively. In some cases, the rep 52 is from AAV2 or the AAV5 with at least about 75%, about 80%, about 85%, about 90%, about 95% or about 99% sequence identity to a wild type rep 52 from AAV2. In some cases, the rep 52 has one or more amino acid substitutions, deletions and/or additions as compared to a wild type rep 52 from AAV1, AAV2, AAV2 variants, AAV3 (including the AAV 3A and 3B), AAV4, AAV5, AAV6, AAV7, AAV8, AAV9, AAV9 variants, AAV10, AAV11, AAV12, AAV13, AAV-Rh10, AAV-Rh74, AAV-2i8, AAV-DJ, LK03, NP59, or KP1. In some cases, the rep 52 has one or more amino acid substitutions, deletions and/or additions as compared to a wild type rep 52 from AAV2 or AAV5. In some cases, the rep 52 has one or more amino acid substitutions, deletions and/or additions as compared to a wild type rep 52 from AAV2.

In some cases, the rep 40 is from AAV1, AAV2, AAV2 variants, AAV3 (including AAV 3A and 3B), AAV4, AAV5, AAV6, AAV7, AAV8, AAV9, AAV9 variants, AAV10, AAV11, AAV12, AAV13, AAV-Rh10, AAV-Rh74, AAV-2i8, AAV-DJ, LK03, NP59, or KP1, or any other AAV. In some cases, the rep 40 is from AAV2 or AAV5. In some cases, the rep 40 is from AAV2. In some cases, the rep 40 is from AAV1, AAV2, AAV2 variants, AAV3 (including AAV 3A and 3B), AAV4, AAV5, AAV6, AAV7, AAV8, AAV9, AAV9 variants, AAV10, AAV11, AAV12, AAV13, AAV-Rh10, AAV-Rh74, AAV-2i8, AAV-DJ, LK03, NP59, or KP1 with at least about 75%, about 80%, about 85%, about 90%, about 95% or about 99% sequence identity to a wild type rep 40 from AAV1, AAV2, AAV2 variants, AAV3 (including AAV 3A and 3B), AAV4, AAV5, AAV6, AAV7, AAV8, AAV9, AAV9 variants, AAV10, AAV11, AAV12, AAV13, AAV-Rh10, AAV-Rh74 or AAV-2i8, AAV-DJ, LK03, NP59, or KP1, respectively. In some cases, the rep 40 is from AAV2 or AAV5 with at least about 75%, about 80%, about 85%, about 90%, about 95% or about 99% sequence identity to a wild type rep 40 from AAV2 or AAV5. In some cases, the rep 40 is from AAV2 or AAV5 with at least about 75%, about 80%, about 85%, about 90%, about 95% or about 99% sequence identity to a wild type rep 40 from AAV2. In some cases, the rep 40 has one or more amino acid substitutions, deletions and/or additions as compared to a wild type rep 40 from AAV1, AAV2, AAV2 variants, AAV3 (including AAV 3A and 3B), AAV4, AAV5, AAV6, AAV7, AAV8, AAV9, AAV9 variants, AAV10, AAV11, AAV12, AAV13, AAV-Rh10, AAV-Rh74, AAV-2i8, AAV-DJ, LK03, NP59, or KP1. In some cases, the rep 40 has a one or more substituted amino acids, deletions and/or additions as compared to a wild type rep 40 from AAV2 or AAV5. In some cases, the rep 40 has a one or more substituted amino acids, deletions and/or additions as compared to a wild type rep 40 from AAV2.

In some cases, the rep protein comprises components from the same serotype AAV, for example, rep 78, rep 68, rep 52 and/or rep 40 from the same serotype AAV. In some cases, the rep protein comprises components from AAV2, for example, rep 78, rep 68, rep 52 and/or rep 40 from AAV2. In some cases, the rep protein comprises components rep 78 and/or rep 52 from AAV2. In some cases, the rep comprises components from AAV5, for example, rep 78, rep 68, rep 52 and/or rep 40 from AAV5. In some cases, the rep protein comprises rep 78 and/or rep 52 from AAV5. In some cases, the rep protein comprises components from different serotypes of AAVs, for example, rep 78, rep 68, rep 52 and/or rep 40 from different serotypes of AAVs. For example, the rep can comprise components from any of rep 78, rep 68, rep 52 and/or rep 40 from AAV1, AAV2, AAV2 variants, AAV3 (including AAV3A and 3B), AAV4, AAV5, AAV6, AAV7, AAV8, AAV9, AAV9 variants, AAV10, AAV11, AV12, AAV13, AAV-Rh10, AAV-Rh74, AAV-2i8, AAV-DJ, LK03, NP59, and/or KP1.

In some cases, the coding sequence of the rep protein comprises a sequence with about 70%, about 75%, about 80%, about 85%, about 90%, about 95%, or about 99% sequence identity to a sequence of SEQ ID NO: 16. In some cases, the coding sequence of the rep protein comprises a sequence with at least 70%, at least 75%, at least 80%, at least 85%, at least 90%, at least 95%, or at least 99% sequence identity to a sequence of SEQ ID NO: 16. In some cases, the coding sequence of the rep protein comprises a sequence of SEQ ID NO: 16. In some cases, the rep protein coding sequence is operably linked to a promoter. The promoter can be any suitable promoter that can drive the expression of the rep protein. In some cases, the promoter is a tissue-specific promoter, a constitutive promoter, or a regulatable promoter. The promoter can be a viral promoter, plant promoter and a mammalian promoter. In some cases, the promoter is a p5 promoter.

### Capsid (Cap) Protein

Disclosed herein are nucleic acid sequences encoding one or more capsid proteins. The nucleic acid sequences can be from a capsid gene region from an AAV. In some cases, the nucleic acid sequences further comprise a promoter operably linked to the capsid coding sequence. The nucleic acid sequences can further comprise a poly(A) sequence operably linked to the 3′ end of the capsid coding sequence. In some cases, the promoter is a Ef1α promoter.

The AAV cap protein can be any structural protein that can form a functional AAV capsid (i.e., packaging DNA and infecting target cells). In some cases, the cap protein includes VP1, VP2, and VP3. In some cases, the cap protein does not need to comprise all of VP1, VP2, and VP3, as long as it can produce a functional AAV capsid. In some cases, the cap protein comprises VP1 and VP2. In some cases, the cap protein comprises VP1 and VP3. In some cases, the cap protein comprises VP2 and VP3. In some cases, the cap protein comprises VP1. In some cases, the cap protein comprises VP2. In some cases, the cap protein comprises VP3.

VP1, VP2, or VP3 can be from any AAV serotype. In some cases, the VP1 is from AAV serotype 1 (AAV1), AAV serotype 2 (AAV2), AAV2 variants, AAV serotype 3 (AAV3, including serotypes 3A and 3B), AAV serotype 4 (AAV4), AAV serotype 5 (AAV5), AAV serotype 6 (AAV6), AAV serotype 7 (AAV7), AAV serotype 8 (AAV8), AAV serotype 9 (AAV9), AAV9 variants, AAV serotype 10 (AAV10), AAV serotype 11 (AAV11), AAV serotype 12 (AAV12), AAV serotype 13 (AAV13), AAV-Rh10, AAV-Rh74, AAV-2i8, AAV-DJ, LK03, NP59, or KP1, or any other AAVs. In some cases, the VP1 is from AAV1, AAV2, AAV2 variants, AAV3, (including AAV3A and 3B), AAV4, AAV5, AAV6, AAV7, AAV8, AAV9, AAV9 variants, AAV10, AAV11, AAV12, AAV13, AAV-Rh10, AAV-Rh74, AAV2i8, AAV-DJ, LK03, NP59, or KP1, with about 75%, about 80%, about 85%, about 90%, about 95%, or about 99% sequence identity to a wildtype VP1 from AAV1, AAV2, AAV2 variants, AAV3, (including AAV3A and 3B), AAV4, AAV5, AAV6, AAV7, AAV8, AAV9, AAV9 variants, AAV10, AAV11, AAV12, AAV13, AAV-Rh10, AAV-Rh74, AAV2i8, AAV-DJ, LK03, NP59, or KP1, respectively. In some cases, the VP1 has one or more amino acid substitutions, deletions, additions, or any combination thereof compared to a wildtype VP1 from AAV1, AAV2, AAV2 variants, AAV3 (including AAV3A and 3B), AAV4, AAV5, AAV6, AAV7, AAV8, of AAV9, AAV9 variants, AAV10, AAV11, AAV12, AAV13, AAV-Rh10, AAV-Rh74, AAV-2i8, AAV-DJ, LK03, NP59, or KP1.

In some cases, the VP2 is from AAV1, AAV2, AAV2 variants, AAV3 (including AAV3A and AAV3B), AAV4, AAV5, AAV6, AAV7, AAV8, AAV9, AAV9 variants, AAV10, AAV11, AAV12, AAV13, AAV-Rh10, AAV-Rh74, AAV-2i8, AAV-DJ, LK03, NP59, or KP1, or any other AAVs. In some cases, the VP2 is from AAV1, AAV2, AAV2 variants, AAV3 (including AAV3A and 3B), AAV4, AAV5, AAV6, AAV7, AAV8, AAV9, AAV9 variants, AAV10, AAV11, AAV12, AAV13, AAV-Rh10, AAV-Rh74, AAV2i8, AAV-DJ, LK03, NP59, or KP1, with about 75%, about 80%, about 85%, about 90%, about 95%, or about 99% sequence identity to a wildtype VP2 from AAV1, AAV2, AAV2 variants, AAV3 (including AAV3A and 3B), AAV4, AAV5, AAV6, AAV7, AAV8, AAV9, AAV9 variants, AAV10, AAV11, AAV12, AAV13, AAV-Rh10, AAV-Rh74, AAV2i8, AAV-DJ, LK03, NP59, or KP1, respectively. In some cases, the VP2 has one or more amino acid substitutions, deletions, additions, or any combination thereof compared to a wildtype VP2 from AAV1, AAV2, AAV2 variants, AAV3 (including AAV3A and 3B), AAV4, AAV5, AAV6, AAV7, AAV8, of AAV9, AAV9 variants, AAV10, AAV11, AAV12, AAV13, AAV-Rh10, AAV-Rh74, AAV-2i8, AAV-DJ, LK03, NP59, or KP1.

The VP3 can be from AAV1, AAV2, AAV2 variants, AAV3 (including AAV3A and AAV3B), AAV4, AAV5, AAV6, AAV7, AAV8, AAV9, AAV9 variants, AAV10, AAV11, AAV12, AAV13, AAV-Rh10, AAV-Rh74, AAV-2i8, AAV-DJ, LK03, NP59, or KP1, or any other AAVs. In some cases, the VP3 is from AAV1, AAV2, AAV2 variants, AAV3, (including AAV3A and 3B), AAV4, AAV5, AAV6, AAV7, AAV8, AAV9, AAV9 variants, AAV10, AAV11, AAV12, AAV13, AAV-Rh10, AAV-Rh74, AAV2i8, AAV-DJ, LK03, NP59, or KP1 with about 75%, about 80%, about 85%, about 90%, about 95%, or about 99% sequence identity to a wildtype VP3 from AAV1, AAV2, AAV2 variants, AAV3, (including AAV3A and 3B), AAV4, AAV5, AAV6, AAV7, AAV8, AAV9, AAV9 variants, AAV10, AAV11, AAV12, AAV13, AAV-Rh10, AAV-Rh74, AAV2i8, AAV-DJ, LK03, NP59, or KP1 respectively. In some cases, the VP3 has one or more amino acid substitutions, deletions, additions, or any combination thereof compared to a wildtype VP3 from AAV1, AAV2, AAV2 variants, AAV3 (including AAV3A and 3B), AAV4, AAV5, AAV6, AAV7, AAV8, AAV9, AAV9 variants, AAV10, AAV11, AAV12, AAV13, AAV-Rh10, AAV-Rh74, AAV-2i8, AAV-DJ, LK03, NP59, or KP1.

In some cases, the cap protein comprises VP1, VP2, VP3, or any combinations thereof from AAV of the same serotype; for example, the cap protein can comprise VP1, VP2, VP3, or any combinations thereof from the same serotype such as AAV1, AAV2, AAV2 variants, AAV3, (including AAV 3A and 3B), AAV4, AAV5, AAV6, AAV7, AAV8, AAV9, AAV9 variants, AAV10, AAV11, AAV12, AAV13, AAV-Rh10, AAV-Rh74, AAV-2i8, AAV-DJ, LK03, NP59, or KP1. In some cases, the cap protein comprises VP1, VP2, VP3, or any combinations thereof from different serotypes of AAV; for example, the cap protein can comprise one or more of VP1, VP2, VP3, or any combination thereof of AAV1, AAV2, AAV2 variants, AAV3 (including AAV3A and 3B), AAV4, AAV5, AAV6, AAV7, AAV8, AAV9, AAV9 variants, AAV10, AAV11, AAV12, AAV13, AAV-Rh10, AAV-Rh74, AAV-2i8, AAV-DJ, LK03, NP59, or KP1, wherein VP1, VP2, VP3 are not from the same serotype.

In some cases, the coding sequence of the capsid comprises the nucleic acid coding sequence of CAP2 or a CAP2 variant. In some cases, the coding sequence of the capsid consists of the nucleic acid coding sequence of CAP2 or a CAP2 variant.

In some embodiments, the cap protein coding sequence is operably linked to a promoter. The promoter can be any suitable promoter that can drive the expression of the cap protein. In some cases, the promoter is a tissue-specific promoter, a constitutive promoter, or a regulatable promoter. The promoter can be a viral promoter, a plant promoter, or a mammalian promoter. In some cases, the promoter is a Ef1α promoter. In some cases, the promoter is a p5 promoter. In some cases, the promoter is a p40 promoter.

In some cases, the capsid and the rep protein are from the same AAV serotype. For example, the capsid and the rep protein can be from the same serotype of AAV1, AAV2, AAV2 variants, AAV3 (including AAV 3A and 3B), AAV4, AAV5, AAV6, AAV7, AAV8, AAV9, AAV9 variants, AAV10, AAV11, AAV12, AAV13, AAV-Rh10, AAV-Rh74, AAV-2i8, AAV-DJ, LK03, NP59, or KP1, or any other AAV. In some cases, the capsid and the rep protein are from the same serotype of AAV2.

In some cases, the capsid and the rep protein are from different AAV serotypes, e.g., the capsid and the rep protein can be respectively from AAV1, AAV2, AAV2 variants, AAV3 (including AAV 3A and 3B), AAV4, AAV5, AAV6, AAV7, AAV8, AAV9, AAV9 variants, AAV10, AAV11, AAV12, AAV13, AAV-Rh 10, AAV-Rh74, AAV-2i8, AAV-DJ, LK03, NP59, or KP1, or any other AAV. For example, in some cases, the rep protein is from AAV2 and the capsid is from AAV5.

In some cases, the AAV particle comprises a capsid from AAV2 and a rep protein from AAV2. In some cases, the AAV particle comprises a capsid from AAV2 and a rep protein from AAV6. In some cases, the AAV particle comprises a capsid from AAV2 and a rep protein from AAV7. In some cases, the AAV particle comprises a capsid from AAV2 and a rep protein from AAV8. In some cases, the AAV particle comprises a capsid from AAV2 and a rep protein from AAV9.

### GOI/Exogenous Gene

A gene of interest (GOI) can be any gene that can be flanked by two ITRs. The GOI flanked by the two ITRs can be referred to as an exogenous gene, a transgene, or a heterologous gene. Disclosed herein are nucleic acid sequences comprising one or more exogenous genes flanked by ITRs. In some cases, the exogenous gene flanked by ITRs comprise one or more genes encoding one or more polypeptides, including one or more polypeptides for therapeutic use. In some cases, the one or more polypeptides for therapeutic use include a protein, an antibody, an intrabody, a contractile protein, an enzyme, a hormonal protein, a structural protein, a storage protein, a small molecule, a protein channel, or a transport protein. Non-limiting examples of enzymes include hydrolase, isomerases, nucleases, ligases, transferases, and oxidoreductases. In some cases, the one or more exogenous genes comprise a gene encoding a peptide or protein that would result in amelioration of symptoms associated with a disease or disorder. Some examples of applicable diseases include Duchenne muscular dystrophy, Spinal Muscular Atrophy, AIDS, Pompe disease, Parkinson's disease, Huntington disease, Alzheimer disease, Gaucher's disease, Hurler's disease, adenosine deaminase (ADA) deficiency, emphysema, progeria, ALS, epilepsy, stroke, hypercholestemia, insulin disorders (e.g., diabetes), growth disorders, various blood disorders (e.g., anemias, thalassemias, hemophilia), genetic defects (e.g., cystic fibrosis), cancer (especially solid tumors), and the like.

The one or more exogenous genes can be any suitable gene for gene therapy, particularly for rAAV gene therapy. In some cases, the one or more exogenous genes flanked by ITRs encodes a human growth hormone (hGH) gene, a RPE65 gene, a LPL gene, a CNGA3 gene, a CNGB3 gene, a mitochondrial DNA, a XLRS gene, a FVIII gene, a FIX gene, an AAT gene, a SMN gene, a MicroDMD gene, an α-Sarcoglycan gene, a Follistatin gene, a dysferlin gene, an NF-κB gene, an IFN-β gene, an ARSA gene, a NGF gene, a Neurturin gene, an AADC gene, a TPP1 gene, a SGSH gene, a SUMF1 gene, and/or an OTC gene.

In some cases, the one or more exogenous genes encode one or more nucleic acid sequences, including one or more modulatory nucleic acid sequences. In some cases, the one or more modulatory nucleic acid sequences can be of therapeutic use, including anti-sense oligonucleotides, siRNA and miRNA.

In some cases, the nucleic acid molecules comprising one or more exogenous genes flanked by ITRs further comprise transcriptional and posttranscriptional regulatory elements. In some cases, the transcriptional and posttranscriptional regulatory elements can modulate (enhance or attenuate) expression of genes delivered by viral vectors. In some cases, the transcriptional and posttranscriptional regulatory elements include a 5′UTR, a promoter (e.g., a CMV promoter), a 3′ UTR, and a poly(A) encoding sequence. In some cases, the transcriptional and posttranscriptional regulatory elements further comprise a stuff DNA (Stuffer), and a Woodchuck Hepatitis Virus posttranscriptional regulatory element (WPRE).

Some nucleotide sequences of AAV ITR regions are known (See e.g., Kotin, Hum. Gene Ther., 5:793-801 [1994]; Berns, “Parvoviridae and Their Replication” in Fields and Knipe (eds), Fundamental Virology, 2nd Edition, for the AAV-2 sequence). AAV ITRs used in the nucleic acid sequences or the vectors of the present disclosure need not have a wild-type nucleotide sequence, and can be altered or modified (e.g., by the insertion, deletion or substitution of nucleotides). Additionally, AAV ITRs can be from any AAV serotypes, including AAV1, AAV2, AAV2 variants, AAV3, (including AAV 3A and 3B), AAV4, AAV5, AAV6, AAV7, AAV8, AAV9, AAV9 variants, AAV10, AAV11, AAV12, AAV13, AAV-Rh10, AAV-Rh74, AAV-2i8, AAV-DJ, LK03, NP59, KP1, and any other AAV serotype. Furthermore, 5′ and 3′ ITRs which flank a selected nucleotide sequence in an AAV vector need not necessarily be identical or from the same AAV serotype or isolate, so long as they function as intended.

In some cases, the exogenous gene has a size between about 1 kb to about 7 kb. In some cases, the exogenous gene has a size between about 2 kb to about 6 kb. In some cases, the exogenous gene has a size between about 2.5 kb to about 5.5 kb. In some cases, the exogenous gene has a size between about 3 kb to about 5 kb. In some cases, the exogenous gene has a size between about 3.5 kb to about 4.5 kb. In some cases, the exogenous gene has a size about 1.5 kb. In some cases, the exogenous gene has a size about 2 kb. In some cases, the exogenous gene has a size about 2.5 kb. In some cases, the exogenous gene has a size about 3 kb. In some cases, the exogenous gene has a size about 3.5 kb. In some cases, the exogenous gene has a size about 4 kb. In some cases, the exogenous gene has a size about 4.5 kb. In some cases, the exogenous gene has a size about 5 kb. In some cases, the exogenous gene has a size about 5.5 kb. In some cases, the exogenous gene has a size about 6 kb. In some cases, the exogenous gene has a size about 2.3 kb. In some cases, the exogenous gene has a size about 4.7 kb. The one or more exogenous gene can have a size from about 1 kilobases to about 10 kilobases, from about 1 kilobases to about 9 kilobases, from about 1 kilobases to about 8 kilobases, from about 2 kilobases to about 7 kilobases, from about 3 kilobases to about 6 kilobases, from about 4 kilobases to about 5 kilobases, from about 1 kilobases to about 5 kilobases, from about 2 kilobases to about 4 kilobases, or from about 2 kilobases to about 5 kilobases. In some cases, the one or more exogenous gene has a size of about 1 kilobases, about 2 kilobases, about 3 kilobases, about 4 kilobases, about 5 kilobases, about 6 kilobases, about 7 kilobases, about 8 kilobases, about 9 kilobases, or about 10 kilobases. In some cases, the one or more exogenous gene has a size larger than 1 kilobases, 2 kilobases, 3 kilobases, 4 kilobases, 5 kilobases, 6 kilobases, 7 kilobases, or 8 kilobases.

### Regulatory Elements

Regulatory elements used in the AAV vectors disclosed herein comprises control sequences. The control sequences can comprise a promoter, such as a CMV promoter, EF1α promoter, or p5 promoter.

A “promoter” can be a DNA sequence located adjacent to a heterologous polynucleotide sequence encoding a target product, which is usually operably linked to an adjacent sequence, such as a coding sequence of a polynucleotide. Compared to the amount expressed in the absence of a promoter, a promoter generally increases the amount of the polynucleotide expression. In some cases, the promoter is adjacent to an exogenous gene. In some cases, the promoter is adjacent to a rep protein coding sequence. In some cases, the promoter is adjacent to a capsid coding sequence. The promoter can be located upstream or downstream of the coding sequence of the polynucleotide. In some cases, the promoter is located upstream of the coding sequence of the polynucleotide, for example, the coding sequence of an exogenous gene, the coding sequence of an AAV rep protein, or the coding sequence of an AAV capsid. In some cases, the promoter is located downstream of the coding sequence of the polynucleotide, for example, the coding sequence of an exogenous gene, the coding sequence of an AAV rep protein, or the coding sequence of an AAV capsid.

Exemplary promoters include AAV p5 promoter, AAV p40 promoter, the phosphoglycerate kinase (PKG) promoter, CAG (composite of the CMV enhancer the chicken beta actin promoter (CBA) and the rabbit beta globin intron.), NSE (neuronal specific enolase), synapsin or NeuN promoters, the SV40 early promoter, mouse mammary tumor virus LTR promoter; adenovirus major late promoter (Ad MLP); a herpes simplex virus (HSV) promoter, a cytomegalovirus (CMV) promoter such as the CMV immediate early promoter region (CMVIE), SFFV promoter, rous sarcoma virus (RSV) promoter, synthetic promoters, hybrid promoters, and the like. Other promoters can be of human origin or from other species, including from mice. Common promoters include, e.g., the human cytomegalovirus (CMV) immediate early gene promoter, the SV40 early promoter, the Rous sarcoma virus long terminal repeat, [beta]-actin, rat insulin promoter, the phosphoglycerate kinase promoter, the human alpha-1 antitrypsin (AAT) promoter, the transthyretin promoter (TTR), the TBG promoter and other liver-specific promoters, the desmin promoter and similar muscle-specific promoters, the EF1-alpha promoter, the CAG promoter and other constitutive promoters, hybrid promoters with multi-tissue specificity, promoters specific for neurons like synapsin and glyceraldehyde-3-phosphate dehydrogenase promoter, at least some of which are promoters well known and readily available to those of skill in the art, can be used to obtain high-level expression of the coding sequence of interest. In addition, sequences from non-viral genes, such as the murine metallothionein gene, can also be used herein. Such promoter sequences are commercially available from, e.g., Stratagene (San Diego, Calif.). Some promoters, such as HLP, LP1, HCR-hAAT, ApoE-hAAT, and LSP, are described in more detail in the following references: HLP: Mcintosh J. et al, Blood 2013 Apr. 25, 121 (17): 3335-44; LP1: Nathwani et al, Blood. 2006 Apr. 1, 107 (7): 2653-2661; HCR-hAAT: Miao et al, Mol Ther. 2000; 1:522-532; ApoE-hAAT: Okuyama et al, Human Gene Therapy, 7, 637-645 (1996); and LSP: Wang et al, Proc Natl Acad Sci USA. 1999 Mar. 30, 96 (7): 3906-3910. Promoter is also described in WO 2011/005968. In some cases, the promoter is a liver specific promoter.

In some cases, the promoter is a promoter suitable for expression in insect cells. In some cases, the suitable promoters for expressing a polynucleotide in an insect cell include a polh promoter, a p10 start promoter, an alkaline promoter, an inducible promoter, an E1 promoter or a Delta E1 promoter.

An “enhancer” is a sequence that enhances the activity of a promoter. Different from the promoter, an enhancer does not have the promoter activity, and may generally depend on its location relative to the promoter (i.e., upstream or downstream of the promoter). Non-limiting examples of enhancer elements (or portions thereof) that can be used in the present disclosure include CMV enhancers, synthetic enhancers, liver-specific enhancers, vascular-specific enhancers, brain-specific enhancers, nerve cell-specific enhancers, lung-specific enhancers, muscle-specific enhancers, kidney-specific enhancers, and pancreas-specific enhancers. Baculovirus enhancers and enhancer elements found in insect cells can also be used herein.

A “stuffer sequence” refers to a nucleotide sequence of a larger nucleic acid molecule (such as, but not limited, to a vector), and is usually to create a desired gap or separation between two nucleic acid features (such as, but not limited, between a promoter and a coding sequence) or to extend the nucleic acid molecule a desired length. The stuffer sequence does not contain protein coding information and may have unknown or synthetic origin, not related to other nucleic acid sequences within the larger nucleic acid molecule, or any combination thereof.

As used herein, the term “intron” generally refers to a DNA molecule that can be isolated or identified from a gene and can be defined generally as a region spliced out during messenger RNA (mRNA) processing prior to translation. Alternately, an intron can be a synthetically produced or manipulated DNA element. An intron can contain enhancer elements that effect the transcription of operably linked genes. An intron can be used as a regulatory element for modulating expression of an operably linked transcribable DNA molecule. A construct can comprise an intron, and the intron may or may not be heterologous with respect to the transcribable DNA molecule.

In some cases, tissue-specific expression may be desirable. Thus, by coupling the coding sequence for a GOI with heterologous control sequences derived from genes that are specifically transcribed by a selected tissue type, a tissue-specific expression of the GOI can be achieved. A number of tissue-specific promoters that enable directed expression in selected tissue types have been described herein.

In some cases, the AAV vector comprises transcriptional and/or posttranscriptional regulatory elements. The transcriptional and/or posttranscriptional regulatory elements can modulate (enhance or attenuate) expression of genes delivered by viral vectors. In some cases, the transcriptional and/or posttranscriptional regulatory elements include a 5′UTR, a promoter (e.g., a CMV promoter), a 3′ UTR, and a poly(A) encoding sequence. In some cases, the transcriptional and/or posttranscriptional regulatory elements further comprise a stuff DNA (Stuffer), and a Woodchuck Hepatitis Virus posttranscriptional regulatory element (WPRE).

In some cases, the 3′ end of a coding polynucleotide sequence, such as, for example, the cap protein coding sequence, the rep protein coding sequence, or the coding sequence of the GOI, further comprises a polyadenylation sequence or “poly A sequence.” In some cases, the polyadenylation sequences or “poly A sequences” can range from about 1 to about 500 base pairs (bp). In some cases, the polyadenylation sequence or “poly A sequence” can be to, 1, 2, 3, 4, 5, 6, 7, 8, 9, 10, 20, 30, 50, 100, 200, or 500 nucleotides.

### Compositions/Two-Vector Systems

Provided herein are compositions used for the two-vector systems as disclosed herein. The composition can comprise two vectors providing elements that can be used for rAAV packaging and production. For example, the composition comprises two vectors encoding (i) an AAV rep protein, (ii) an AAV capsid, (iii) at least one helper factor, and (iv) an exogenous gene (GOI). In some cases, there is no additional vector in the composition other than the two vectors. In some cases, the vector is a plasmid.

In some cases, the composition comprises two plasmids, including a first plasmid comprising a first nucleic acid sequence comprising a sequence encoding a rep protein and a sequence encoding a capsid, and a second nucleic acid sequence comprising an exogenous gene, and a second plasmid comprising at least one helper element.

In some cases, the composition comprises two plasmids, including a first plasmid comprising a first nucleic acid sequences comprising a sequence encoding a rep protein and a sequence encoding a capsid, and a second nucleic acid sequence comprising at least one helper element, and a second plasmid comprising an exogenous gene.

In some cases, the composition comprises two plasmids, including a first plasmid comprises a first nucleic acid sequence comprising a sequence encoding a capsid and a second nucleic acid sequence comprising an exogenous gene, and a second plasmid comprising a first nucleic acid sequence comprising a sequence encoding a rep protein and a second nucleic acid sequence comprising at least one helper element; wherein the second plasmid does not comprise a sequence encoding a capsid. In some cases, the first plasmid does not comprise a sequence encoding a rep protein.

In some cases, the composition comprises two plasmids, including a first plasmid comprising a first nucleic acid sequence comprising a sequence encoding a rep protein and a second nucleic acid sequence comprising an exogenous gene; and a second plasmid comprising a first nucleic acid sequence comprising a sequence encoding a capsid and a second nucleic acid sequence comprising at least one helper element; wherein the second plasmid does not comprise a sequence encoding a rep protein. In some cases, the first plasmid does not comprise a sequence encoding a capsid.

In some cases, the composition comprises two plasmids, including a first plasmid comprising (i) a sequence encoding an AAV rep protein and an AAV capsid and (ii) at least one helper element, and a second plasmid comprising an exogenous gene (GOI). In some cases, the composition comprises two plasmids, including a first plasmid comprising (i) a sequence encoding an AAV rep protein and an AAV capsid and (ii) an exogenous gene (GOI), and a second plasmid comprising at least one helper element. In some cases, the composition comprises two plasmids, including a first plasmid comprising (i) a sequencing encoding an AAV capsid and (ii) an exogenous gene (GOI), and a second plasmid comprising (i) a sequence encoding an AAV rep protein and (ii) at least one helper element. In some cases, the composition comprises two plasmids, including a first plasmid comprising (i) a sequence encoding an AAV rep protein and (ii) an exogenous gene (GOI), and a second plasmid comprising (i) a sequence encoding an AAV capsid and (ii) at least one helper element.

In some cases, the composition comprises two plasmids, including a first plasmid comprising a sequence with about 70%, about 75%, about 80%, about 85%, about 90%, about 95%, or about 99% sequence identity to SEQ ID NO: 7 and a second plasmid comprising an exogenous gene. In some cases, the composition comprises two plasmids, including a first plasmid comprises a sequence with at least 70%, at least 75%, at least 80%, at least 85%, at least 90%, at least 95%, or at least 99% sequence identity to SEQ ID NO: 7 and a second plasmid comprising an exogenous gene. In some cases, the composition comprises two plasmids, including a first plasmid comprising a sequence of SEQ ID NO: 7 and a second plasmid comprising an exogenous gene. In some cases, the first plasmid further comprises a sequence encoding an AAV capsid. In some cases, the first plasmid further comprises a sequence encoding an AAV rep protein. In some cases, the rep protein is from AAV2.

In some cases, the composition comprises two plasmids, including a first plasmid comprising a sequence with about 70%, about 75%, about 80%, about 85%, about 90%, about 95%, or about 99% sequence identity to SEQ ID NO: 12 and a second plasmid comprising an exogenous gene. In some cases, the composition comprises two plasmids, including a first plasmid comprising a sequence with at least 70%, at least 75%, at least 80%, at least 85%, at least 90%, at least 95%, or at least 99% sequence identity to SEQ ID NO: 12 and a second plasmid comprising an exogenous gene. In some cases, the composition comprises two plasmids, including a first plasmid comprising a sequence of SEQ ID NO: 12 and a second plasmid comprising an exogenous gene. In some cases, the first plasmid further comprises at least one helper element. In some cases, the at least one helper element is E4, E2A, or VA. In some cases, the at least one helper element comprises or consists of E4, E2A, and VA. In some cases, the at least one helper element is from Ad5.

In some cases, the composition comprises two plasmids, including a first plasmid comprising a sequence with about 70%, about 75%, about 80%, about 85%, about 90%, about 95%, or about 99% sequence identity to SEQ ID NO: 11 and a second plasmid comprising an exogenous gene. In some cases, the composition comprises two plasmids, including a first plasmid comprising a sequence with at least 70%, at least 75%, at least 80%, at least 85%, at least 90%, at least 95%, or at least 99% sequence identity to SEQ ID NO: 11 and a second plasmid comprising an exogenous gene. In some cases, the composition comprises two plasmids, including a first plasmid comprising a sequence of SEQ ID NO: 11 and a second plasmid comprising an exogenous gene. In some cases, the first plasmid further comprises at least one helper element. In some cases, the at least one helper element is E4, E2A, or VA. In some cases, the at least one helper element comprises or consists of E4, E2A, and VA. In some cases, the at least one helper element is from Ad5.

In some cases, the composition comprises two plasmids, including a first plasmid comprising or consisting of a sequence with about 70%, about 75%, about 80%, about 85%, about 90%, about 95%, or about 99% sequence identity to SEQ ID NO: 3 and a second plasmid comprising an exogenous gene. In some cases, the composition comprises two plasmids, including a first plasmid comprising or consisting of a sequence with at least 70%, at least 75%, at least 80%, at least 85%, at least 90%, at least 95%, or at least 99% sequence identity to SEQ ID NO: 3 and a second plasmid comprising an exogenous gene. In some cases, the composition comprises two plasmids, including a first plasmid comprising or consisting of a sequence of SEQ ID NO: 3 and a second plasmid comprising an exogenous gene.

In some cases, the composition comprises two plasmids, including a first plasmid comprising or consisting of a sequence with about 70%, about 75%, about 80%, about 85%, about 90%, about 95%, or about 99% sequence identity to SEQ ID NO: 1 and a second plasmid comprising an exogenous gene. In some cases, the composition comprises two plasmids, including a first plasmid comprising or consisting of a sequence with at least 70%, at least 75%, at least 80%, at least 85%, at least 90%, at least 95%, or at least 99% sequence identity to SEQ ID NO: 1 and a second plasmid comprising an exogenous gene. In some cases, the composition comprises two plasmids, including a first plasmid comprising or consisting of a sequence of SEQ ID NO: 1 and a second plasmid comprising an exogenous gene.

In some cases, the composition comprises two plasmids, including a first plasmid comprises a sequence with about 70%, about 75%, about 80%, about 85%, about 90%, about 95%, or about 99% sequence identity to SEQ ID NO: 7 and a second plasmid comprises an exogenous gene and a capsid coding sequence. In some cases, the composition comprises two plasmids, including a first plasmid comprising a sequence with at least 70%, at least 75%, at least 80%, at least 85%, at least 90%, at least 95%, or at least 99% sequence identity to SEQ ID NO: 7 and a second plasmid comprising an exogenous gene and a capsid coding sequence. In some cases, the composition comprises two plasmids, including a first plasmid comprising a sequence of SEQ ID NO: 7 and a second plasmid comprising an exogenous gene and a capsid coding sequence. In some cases, the first plasmid further comprises a rep protein. In some cases, the second plasmid further comprises a rep protein. The rep protein can be any rep protein disclosed herein. In some cases, the rep protein is from AAV2.

In some cases, the composition comprises two plasmids, including a first plasmid comprising a sequence with about 70%, about 75%, about 80%, about 85%, about 90%, about 95%, or about 99% sequence identity to SEQ ID NO: 13 and a second plasmid comprising an exogenous gene and a capsid coding sequence. In some cases, the composition comprises two plasmids, including a first plasmid comprising a sequence with at least 70%, at least 75%, at least 80%, at least 85%, at least 90%, at least 95%, or at least 99% sequence identity to SEQ ID NO: 13 and a second plasmid comprising an exogenous gene and a capsid coding sequence. In some cases, the composition comprises two plasmids, including a first plasmid comprising a sequence of SEQ ID NO: 13 and a second plasmid comprising an exogenous gene and a capsid coding sequence. In some cases, the first plasmid further comprises at least one helper element. In some cases, the at least one helper element is E4, E2A, or VA. In some cases, the at least one helper element comprises or consists of E4, E2A, and VA. In some cases, the at least one helper element is from Ad5 or Ad2.

In some cases, the composition comprises two plasmids, including a first plasmid comprising a sequence with about 70%, about 75%, about 80%, about 85%, about 90%, about 95%, or about 99% sequence identity to SEQ ID NO: 13 and an exogenous gene and a second plasmid comprising a capsid coding sequence and at least one helper element. In some cases, the composition comprises two plasmids, including a first plasmid comprising a sequence with at least 70%, at least 75%, at least 80%, at least 85%, at least 90%, at least 95%, or at least 99% sequence identity to SEQ ID NO: 13 and an exogenous gene and a second plasmid comprising a capsid coding sequence and at least one helper element. In some cases, the composition comprises two plasmids, including a first plasmid comprising a sequence of SEQ ID NO: 13 and an exogenous gene and a second plasmid comprising a capsid coding sequence and at least one helper element. In some cases, the at least one helper element is E4, E2A, or VA. In some cases, the at least one helper element comprises or consists of E4, E2A, and VA. In some cases, the at least one helper element is from Ad5 or Ad2. In some cases, the at least one helper element is from Ad5.

In some cases, the composition comprises two plasmids, including a first plasmid comprising or consisting of a sequence with about 70%, about 75%, about 80%, about 85%, about 90%, about 95%, or about 99% sequence identity to SEQ ID NO: 2 and a second plasmid comprising an exogenous gene and a capsid coding sequence. In some cases, the composition comprises two plasmids, including a first plasmid comprising or consisting of a sequence with at least 70%, at least 75%, at least 80%, at least 85%, at least 90%, at least 95%, or at least 99% sequence identity to SEQ ID NO: 2 and a second plasmid comprising an exogenous gene and a capsid coding sequence. In some cases, the composition comprises two plasmids, including a first plasmid comprising or consisting of a sequence of SEQ ID NO: 2 and a second plasmid comprising an exogenous gene and a capsid coding sequence.

In some cases, the composition comprises two plasmids, including a first plasmid comprising a sequence with about 70%, about 75%, about 80%, about 85%, about 90%, about 95%, or about 99% sequence identity to SEQ ID NO: 14 or 18 and a second plasmid comprising an exogenous gene and a rep protein coding sequence. In some cases, the composition comprises two plasmids, including a first plasmid comprising a sequence with at least 70%, at least 75%, at least 80%, at least 85%, at least 90%, at least 95%, or at least 99% sequence identity to SEQ ID NO: 14 or 18 and a second plasmid comprising an exogenous gene and a rep protein coding sequence. In some cases, the composition comprises two plasmids, including a first plasmid comprising a sequence of SEQ ID NO: 14 or 18 and a second plasmid comprising an exogenous gene and a rep protein coding sequence. In some cases, the first plasmid comprises at least one helper element. In some cases, the at least one helper element is E4, E2A, or VA. In some cases, the at least one helper element comprises or consists of E4, E2A, and VA. In some cases, the at least one helper element is from Ad5 or Ad2. In some cases, the at least one helper element is from Ad5.

In some cases, the composition comprises two plasmids, including a first plasmid comprising a sequence with about 70%, about 75%, about 80%, about 85%, about 90%, about 95%, or about 99% sequence identity to SEQ ID NO: 14 or 18 and a second plasmid comprising a rep protein coding sequence and at least one helper element. In some cases, the composition comprises two plasmids, including a first plasmid comprising a sequence with at least 70%, at least 75%, at least 80%, at least 85%, at least 90%, at least 95%, or at least 99% sequence identity to SEQ ID NO: 14 or 18 and a second plasmid comprising a rep protein coding sequence and at least one helper element. In some cases, the composition comprises two plasmids, including a first plasmid comprising a sequence of SEQ ID NO: 14 or 18 and a second plasmid comprising a rep protein coding sequence and at least one helper element. In some cases, the first plasmid further comprises an exogenous gene. In some cases, the at least one helper element is E4, E2A, or VA. In some cases, the at least one helper element comprises or consists of E4, E2A, and VA. In some cases, the at least one helper element is from Ad5 or Ad2. In some cases, the at least one helper element is from Ad5.

In some cases, the composition comprises two plasmids, including a first plasmid comprising a rep coding sequence and a helper coding sequence, and a second plasmid comprising or consisting of a sequence with about 70%, about 75%, about 80%, about 85%, about 90%, about 95%, or about 99% sequence identity to SEQ ID NO: 15 or 17. In some cases, the composition comprises two plasmids, including a first plasmid comprising a rep coding sequence and a helper coding sequence, and a second plasmid comprising or consisting of a sequence with at least 70%, at least 75%, at least 80%, at least 85%, at least 90%, at least 95%, or at least 99% sequence identity to SEQ ID NO: 15 or 17. In some cases, the composition comprises two plasmids, including a first plasmid comprising a rep coding sequence and a helper coding sequence, and a second plasmid comprising or consisting of a sequence of SEQ ID NO: 15 or 17.

In some cases, the composition comprises two plasmids, including a first plasmid comprising or consisting of a sequence with about 70%, about 75%, about 80%, about 85%, about 90%, about 95%, or about 99% sequence identity to SEQ ID NO: 4 and a second plasmid comprising at least one helper element. In some cases, the composition comprises two plasmids, including a first plasmid comprising or consisting of a sequence with at least 70%, at least 75%, at least 80%, at least 85%, at least 90%, at least 95%, or at least 99% sequence identity to SEQ ID NO: 4 and a second plasmid comprising at least one helper element. In some cases, the composition comprises two plasmids, including a first plasmid comprising or consisting of a sequence of SEQ ID NO: 4 and a second plasmid comprising at least one helper element. In some cases, the second plasmid comprises a sequence with about 70%, about 75%, about 80%, about 85%, about 90%, about 95%, or about 99% sequence identity to SEQ ID NO: 7. In some cases, the second plasmid comprises a sequence with at least 70%, at least 75%, at least 80%, at least 85%, at least 90%, at least 95%, or at least 99% sequence identity to SEQ ID NO: 7.

In some cases, the composition comprises two plasmids, including a first plasmid comprising or consisting of a sequence with about 70%, about 75%, about 80%, about 85%, about 90%, about 95%, or about 99% sequence identity to SEQ ID NO: 5 and a second plasmid comprising at least one helper element. In some cases, the composition comprises two plasmids, including a first plasmid comprising or consisting of a sequence with at least 70%, at least 75%, at least 80%, at least 85%, at least 90%, at least 95%, or at least 99% sequence identity to SEQ ID NO: 5 and a second plasmid comprising at least one helper element. In some cases, the composition comprises two plasmids, including a first plasmid comprising or consisting of a sequence of SEQ ID NO: 5 and a second plasmid comprising at least one helper element. In some cases, the second plasmid comprises a sequence with about 70%, about 75%, about 80%, about 85%, about 90%, about 95%, or about 99% sequence identity to SEQ ID NO: 7. In some cases, the second plasmid comprises a sequence with at least 70%, at least 75%, at least 80%, at least 85%, at least 90%, at least 95%, or at least 99% sequence identity to SEQ ID NO: 7.

In some cases, the composition comprises two plasmids. The first plasmid can comprise a) a first sequence comprising a sequence encoding an AAV rep protein and a sequence encoding an AAV capsid, wherein the isolated nucleic acid molecule lacks a promoter upstream of the sequence encoding the AAV rep protein; and b) a second sequence comprising one or more helper element. In some cases, the AAV rep protein is from AAV2. The AAV capsid can be from AAV1, AAV2, AAV2 variants, AAV3 (including AAV 3A and 3B), AAV4, AAV5, AAV6, AAV7, AAV8, AAV9, AAV9 variants, AAV10, AAV11, AAV12, AAV13, AAV-Rh10, AAV-Rh74, AAV-2i8, AAV-DJ, LK03, NP59, or KP1, or any other AAV. In some cases, the AAV capsid is from AAV2, AAV6, AAV7, AAV8, or AAV9. In some cases, the first sequence comprises a promoter downstream of the sequence encoding the capsid. The promoter can be operably linked to the 3′ end of the sequence encoding the capsid. In some cases, the first sequence comprises a poly(A) sequence at the 3′ end of the sequence encoding the capsid. In some cases, the promoter is operably linked to the 3′ end of the poly(A) sequence. In some cases, the promoter is a p5 promoter. The one or more helper element can be any element having the helper function for rAAV packaging. In some cases, the one or more helper element is VA gene, E2A(DBP) gene, or E4 gene. In some cases, the one or more helper element is from Ad2 or Ad5. In some cases, the rAAV vectors can be constructed to provide, as operatively linked components, a first sequence comprising an AAV rep protein coding sequence, an AAV capsid coding sequence, a poly(A) sequence, a promoter; and a second sequence comprising VA gene, E2A(DBP) gene, and E4 gene. In some cases, the promoter is a p5 promoter.

In some cases, the first plasmid comprises a nucleic acid sequence with about 70%, about 75%, about 80%, about 85%, about 90%, about 95%, about 96%, about 97%, about 98%, about 99%, about 99.5%, or about 99.9% sequence identity to SEQ ID NO: 7. In some cases, the first plasmid comprises a nucleic acid sequence with at least 70%, at least 75%, at least 80%, at least 85%, at least 90%, at least 95%, at least 96%, at least 97%, at least 98%, at least 99%, at least 99.5%, or at least 99.9% sequence identity to SEQ ID NO: 7. In some cases, the first plasmid comprises a sequence of SEQ ID NO: 7. In some cases, the first plasmid comprises a nucleic acid sequence with about 70%, about 75%, about 80%, about 85%, about 90%, about 95%, about 96%, about 97%, about 98%, about 99%, about 99.5%, or about 99.9% sequence identity to SEQ ID NO: 12. In some cases, the first plasmid comprises a nucleic acid sequence with at least 70%, at least 75%, at least 80%, at least 85%, at least 90%, at least 95%, at least 96%, at least 97%, at least 98%, at least 99%, at least 99.5%, or at least 99.9% sequence identity to SEQ ID NO: 12. In some cases, the first plasmid comprises a sequence of SEQ ID NO: 12. In some cases, the first plasmid comprises or consists of a sequence with about 70%, about 75%, about 80%, about 85%, about 90%, about 95%, about 96%, about 97%, about 98%, about 99%, about 99.5%, or about 99.9% sequence identity to SEQ ID NO: 1. In some cases, the first plasmid comprises or consists of a sequence with at least 70%, at least 75%, at least 80%, at least 85%, at least 90%, at least 95%, at least 96%, at least 97%, at least 98%, at least 99%, at least 99.5%, or at least 99.9% sequence identity to SEQ ID NO: 1. In some cases, the first plasmid comprises or consists of a sequence of SEQ ID NO: 1.

The second plasmid can comprise an exogenous gene (GOI). The GOI can be flanked by two ITRs. In some cases, the exogenous gene flanked by ITRs comprise one or more genes encoding one or more polypeptides, including one or more polypeptides for therapeutic use. In some cases, the one or more polypeptides for therapeutic use include a protein, an antibody, an intrabody, a contractile protein, an enzyme, a hormonal protein, a structural protein, a storage protein, a small molecule, a protein channel, or a transport protein. Non-limiting examples of enzymes include hydrolase, isomerases, nucleases, ligases, transferases, and oxidoreductases. In some cases, the one or more exogenous genes flanked by ITRs encodes a human growth hormone (hGH) gene. In some cases, the hGH gene comprises or consists of a sequence of SEQ ID NO: 6. In some cases, the hGH gene comprises or consists of a sequence with about 70%, about 75%, about 80%, about 85%, about 90%, about 95%, or about 99% of sequence identity to SEQ ID NO: 6. In some cases, the hGH gene comprises or consists of a sequence with at least 70%, at least 75%, at least 80%, at least 85%, at least 90%, at least 95%, or at least 99% of sequence identity to SEQ ID NO: 6. In some cases, the one or more exogenous genes encode one or more nucleic acid sequences, including one or more modulatory nucleic acid sequences. In some cases, the one or more modulatory nucleic acid sequences can be of therapeutic use, including anti-sense oligonucleotides, siRNA and miRNA. In some cases, the second plasmid further comprise transcriptional and posttranscriptional regulatory elements. In some cases, the transcriptional and posttranscriptional regulatory elements can modulate (enhance or attenuate) expression of genes delivered by viral vectors. In some cases, the transcriptional and posttranscriptional regulatory elements include a 5′UTR, a promoter (e.g., a CMV promoter), a 3′ UTR, and a poly(A) coding sequence. In some cases, the transcriptional and posttranscriptional regulatory elements further comprise a stuff DNA (Stuffer), and a Woodchuck Hepatitis Virus posttranscriptional regulatory element (WPRE).

In some cases, the composition comprises two plasmids. The first plasmid can comprise a) a first sequence comprising a sequence encoding an AAV rep protein and b) a second sequence comprises one or more helper element; wherein the first sequence comprises a non-AUG start codon upstream of the sequence encoding the AAV rep protein. In some cases, the non-AUG translation start codon is ACG. The AAV rep protein can be from AAV1, AAV2, AAV2 variants, AAV3 (including AAV 3A and 3B), AAV4, AAV5, AAV6, AAV7, AAV8, AAV9, AAV9 variants, AAV10, AAV11, AAV12, AAV13, AAV-Rh10, AAV-Rh74, AAV-2i8, AAV-DJ, LK03, NP59, or KP1, or any other AAV. In some cases, the AAV rep protein is from AAV2. In some cases, the non-AUC start codon is upstream of the sequence encoding an AAV rep protein. The non-AUC start codon can be at the 5′ end of the sequence encoding an AAV rep protein. In some cases, the first sequence comprises a promoter upstream of the sequence encoding the rep protein. The promoter can be operably linked to the 5′ end of the sequence encoding the rep protein. In some cases, the promoter is a p5 promoter. In some cases, the first sequence comprises a poly(A) sequence. The poly(A) sequence can be operably linked to the 3′ end of the rep protein coding sequence. The one or more helper element can be any element having the helper function for rAAV packaging. In some cases, the one or more helper element is VA gene, E2A(DBP) gene, or E4 gene. In some cases, the one or more helper element is from Ad2 or Ad5. The first plasmid can be constructed to provide, as operatively linked components, a first sequence comprising a promoter, a non-AUG start codon, a rep protein coding sequence, a poly(A) sequence; and a second sequence comprising VA gene, E2A(DBP) gene, and E4 gene. In some cases, the promoter is a p5 promoter. In some cases, the non-AUG start codon is ACG.

In some cases, the first plasmid comprises a nucleic acid sequence with about 70%, about 75%, about 80%, about 85%, about 90%, about 95%, about 96%, about 97%, about 98%, about 99%, about 99.5%, or about 99.9% sequence identity to SEQ ID NO: 7. In some cases, the first plasmid comprises a nucleic acid sequence with at least 70%, at least 75%, at least 80%, at least 85%, at least 90%, at least 95%, at least 96%, at least 97%, at least 98%, at least 99%, at least 99.5%, or at least 99.9% sequence identity to SEQ ID NO: 7. In some cases, the first plasmid comprises a sequence of SEQ ID NO: 7. In some cases, the first plasmid comprises a nucleic acid sequence with about 70%, about 75%, about 80%, about 85%, about 90%, about 95%, about 96%, about 97%, about 98%, about 99%, about 99.5%, or about 99.9% sequence identity to SEQ ID NO: 13. In some cases, the first plasmid comprises a nucleic acid sequence with at least 70%, at least 75%, at least 80%, at least 85%, at least 90%, at least 95%, at least 96%, at least 97%, at least 98%, at least 99%, at least 99.5%, or at least 99.9% sequence identity to SEQ ID NO: 13. In some cases, the first plasmid comprises a sequence of SEQ ID NO: 13. In some cases, the first plasmid comprises or consists of a sequence with about 70%, about 75%, about 80%, about 85%, about 90%, about 95%, about 96%, about 97%, about 98%, about 99%, about 99.5%, or about 99.9% sequence identity to SEQ ID NO: 2. In some cases, the first plasmid comprises or consists of a sequence with at least 70%, at least 75%, at least 80%, at least 85%, at least 90%, at least 95%, at least 96%, at least 97%, at least 98%, at least 99%, at least 99.5%, or at least 99.9% sequence identity to SEQ ID NO: 2. In some cases, the first plasmid comprises or consists of a sequence of SEQ ID NO: 2.

The second plasmid can comprise a) a first sequence comprising a sequence encoding an AAV capsid; and b) a second sequence comprising an exogenous gene flanked by inverted terminal repeat (ITR) sequences; wherein the isolated nucleic acid molecule does not comprise a sequence encoding an AAV rep protein. In some cases, the first sequence comprises a promoter upstream of the sequence encoding the AAV capsid. In some cases, the promoter is Ef1α promoter. The promoter can be operably linked to the capsid coding sequence. The AAV capsid can be from AAV1, AAV2, AAV2 variants, AAV3 (including AAV 3A and 3B), AAV4, AAV5, AAV6, AAV7, AAV8, AAV9, AAV9 variants, AAV10, AAV11, AAV12, AAV13, AAV-Rh10, AAV-Rh74, AAV-2i8, AAV-DJ, LK03, NP59, or KP1, or any other AAV. In some cases, the AAV capsid is from AAV2, AAV6, AAV7, AAV8, or AAV9. In some cases, the second sequence comprises a promoter, a post-transcriptional regulatory element, or a poly(A) sequence. In some cases, the promoter is a CMV promoter. The promoter can be operably linked to the exogenous gene. In some cases, the post-transcriptional regulatory element is WPRE. The post-transcriptional regulatory element can be operably linked to the exogenous gene. The second plasmid can be constructed to provide, as operatively linked components, a first sequence comprising promoter, a capsid coding sequence, and a poly(A) sequence; and a second sequence comprising a 5′ ITR, a promoter, a GOI coding sequence, a WPRE, a poly(A) sequence, and a 3′ ITR. In some cases, the promoter in the first sequence is a Ef1α promoter. In some cases, the promoter in the second sequence is a CMV promoter.

In some cases, the second plasmid comprises a nucleic acid sequence with about 70%, about 75%, about 80%, about 85%, about 90%, about 95%, about 96%, about 97%, about 98%, about 99%, about 99.5%, or about 99.9% sequence identity to SEQ ID NO: 6. In some cases, the second plasmid comprises a nucleic acid sequence with at least 70%, at least 75%, at least 80%, at least 85%, at least 90%, at least 95%, at least 96%, at least 97%, at least 98%, at least 99%, at least 99.5%, or at least 99.9% sequence identity to SEQ ID NO: 6. In some cases, the second plasmid comprises a sequence of SEQ ID NO: 6 In some cases, the second plasmid comprises a nucleic acid sequence with about 70%, about 75%, about 80%, about 85%, about 90%, about 95%, about 96%, about 97%, about 98%, about 99%, about 99.5%, or about 99.9% sequence identity to SEQ ID NO: 14 or 18. In some cases, the second plasmid comprises a nucleic acid sequence with at least 70%, at least 75%, at least 80%, at least 85%, at least 90%, at least 95%, at least 96%, at least 97%, at least 98%, at least 99%, at least 99.5%, or at least 99.9% sequence identity to SEQ ID NO: 14 or 18. In some cases, the second plasmid comprises a sequence of SEQ ID NO: 14 or 18. In some cases, the second plasmid comprises or consists of a sequence with about 70%, about 75%, about 80%, about 85%, about 90%, about 95%, about 96%, about 97%, about 98%, about 99%, about 99.5%, or about 99.9% sequence identity to SEQ ID NO:15 or 17. In some cases, the second plasmid comprises or consists of a sequence with at least 70%, at least 75%, at least 80%, at least 85%, at least 90%, at least 95%, at least 96%, at least 97%, at least 98%, at least 99%, at least 99.5%, or at least 99.9% sequence identity to SEQ ID NO: 15 or 17. In some cases, the second plasmid comprises or consists of a sequence of SEQ ID NO: 15 or 17.

Further disclosed herein is a composition comprising two plasmids. The first plasmid comprises a) a first sequence comprising a sequence encoding an AAV rep protein and a sequence encoding an AAV capsid; and b) a second sequence comprises one or more helper element; wherein the first sequence comprises a non-AUG start codon upstream of the sequence encoding the AAV rep protein. In some cases, the non-AUG translation start codon is ACG. In some cases, the AAV rep protein is from AAV2. The AAV capsid can be from AAV1, AAV2, AAV2 variants, AAV3 (including AAV 3A and 3B), AAV4, AAV5, AAV6, AAV7, AAV8, AAV9, AAV9 variants, AAV10, AAV11, AAV12, AAV13, AAV-Rh10, AAV-Rh74, AAV-2i8, AAV-DJ, LK03, NP59, or KP1, or any other AAV. In some cases, the AAV capsid is from AAV2, AAV6, AAV7, AAV8, or AAV9. In some cases, the non-AUC start codon is upstream of the sequence encoding an AAV rep protein. The non-AUC start codon can be at the 5′ end of the sequence encoding an AAV rep protein. In some cases, the first sequence comprises a promoter upstream of the sequence encoding the rep protein. The promoter can be operably linked to the 5′ end of the sequence encoding the rep protein. In some cases, the promoter is a p5 promoter. In some cases, the first sequence comprises a poly(A) sequence. The poly(A) sequence can be operably linked to the 3′ end of the capsid coding sequence. The one or more helper element can be any element having the helper function for rAAV packaging. In some cases, the one or more helper element is VA gene, E2A(DBP) gene, or E4 gene. In some cases, the one or more helper element is from Ad2 or Ad5. The first plasmid can be constructed to provide, as operatively linked components, a first sequence comprising a promoter, a non-AUG start codon, a rep protein coding sequence, a capsid coding sequence, a poly(A) sequence; and a second sequence comprising VA gene, E2A(DBP) gene, and E4 gene. In some cases, the promoter is a p5 promoter. In some cases, the non-AUG start codon is ACG.

In some cases, the first plasmid comprises a nucleic acid sequence with about 70%, about 75%, about 80%, about 85%, about 90%, about 95%, about 96%, about 97%, about 98%, about 99%, about 99.5%, or about 99.9% sequence identity to SEQ ID NO: 7. In some cases, the first plasmid comprises a nucleic acid sequence with at least 70%, at least 75%, at least 80%, at least 85%, at least 90%, at least 95%, at least 96%, at least 97%, at least 98%, at least 99%, at least 99.5%, or at least 99.9% sequence identity to SEQ ID NO: 7. In some cases, the first plasmid comprises a sequence of SEQ ID NO: 7. In some cases, the first plasmid comprises a nucleic acid sequence with about 70%, about 75%, about 80%, about 85%, about 90%, about 95%, about 96%, about 97%, about 98%, about 99%, about 99.5%, or about 99.9% sequence identity to SEQ ID NO: 11. In some cases, the first plasmid comprises a nucleic acid sequence with at least 70%, at least 75%, at least 80%, at least 85%, at least 90%, at least 95%, at least 96%, at least 97%, at least 98%, at least 99%, at least 99.5%, or at least 99.9% sequence identity to SEQ ID NO: 11. In some cases, the first plasmid comprises a sequence of SEQ ID NO: 11. In some cases, the first plasmid comprises or consists of a sequence with about 70%, about 75%, about 80%, about 85%, about 90%, about 95%, about 96%, about 97%, about 98%, about 99%, about 99.5%, or about 99.9% sequence identity to SEQ ID NO: 3. In some cases, the first plasmid comprises or consists of a sequence with at least 70%, at least 75%, at least 80%, at least 85%, at least 90%, at least 95%, at least 96%, at least 97%, at least 98%, at least 99%, at least 99.5%, or at least 99.9% sequence identity to SEQ ID NO: 3. In some cases, the first plasmid comprises or consists of a sequence of SEQ ID NO: 3.

The second plasmid can comprise an exogenous gene (GOI). The GOI can be flanked by two ITRs. In some cases, the exogenous gene flanked by ITRs comprise one or more genes encoding one or more polypeptides, including one or more polypeptides for therapeutic use. In some cases, the one or more polypeptides for therapeutic use include a protein, an antibody, an intrabody, a contractile protein, an enzyme, a hormonal protein, a structural protein, a storage protein, a small molecule, a protein channel, or a transport protein. Non-limiting examples of enzymes include hydrolase, isomerases, nucleases, ligases, transferases, and oxidoreductases. In some cases, the one or more exogenous genes flanked by ITRs encodes a human growth hormone (hGH) gene. In some cases, the hGH gene comprises or consists of a sequence of SEQ ID NO: 6. In some cases, the hGH gene comprises or consists of a sequence with about 70%, about 75%, about 80%, about 85%, about 90%, about 95%, or about 99% of sequence identity to SEQ ID NO: 6. In some cases, the hGH gene comprises or consists of a sequence with at least 70%, at least 75%, at least 80%, at least 85%, at least 90%, at least 95%, or at least 99% of sequence identity to SEQ ID NO: 6. In some cases, the one or more exogenous genes encode one or more nucleic acid sequences, including one or more modulatory nucleic acid sequences. In some cases, the one or more modulatory nucleic acid sequences can be of therapeutic use, including anti-sense oligonucleotides, siRNA and miRNA. In some cases, the second plasmid further comprise transcriptional and posttranscriptional regulatory elements. In some cases, the transcriptional and posttranscriptional regulatory elements can modulate (enhance or attenuate) expression of genes delivered by viral vectors. In some cases, the transcriptional and posttranscriptional regulatory elements include a 5′UTR, a promoter (e.g., a CMV promoter), a 3′ UTR, and a poly(A) coding sequence. In some cases, the transcriptional and posttranscriptional regulatory elements further comprise a stuff DNA (Stuffer), and a Woodchuck Hepatitis Virus posttranscriptional regulatory element (WPRE).

Disclosed herein is a composition comprising two plasmids. The first plasmid can comprise a first sequence comprising a sequence encoding an AAV rep protein and a sequence encoding an AAV capsid; and b) a second sequence comprising an exogenous gene flanked by inverted terminal repeat (ITR) sequences; wherein the first sequence comprises a non-AUG start codon. In some cases, the non-AUG start codon is ACG. In some cases, the non-AUC start codon is upstream of the sequence encoding an AAV rep protein. In some cases, the non-AUC start codon is at 5′ end of the rep protein coding sequence. In some cases, the first sequence comprises a promoter upstream of the sequence encoding the rep protein. In some cases, the promoter is p5 promoter. In some cases, the first sequence comprises a poly(A) sequence. The poly(A) sequence can be located at the 3′ end of the capsid coding sequence. The AAV capsid can be from AAV1, AAV2, AAV2 variants, AAV3 (including AAV 3A and 3B), AAV4, AAV5, AAV6, AAV7, AAV8, AAV9, AAV9 variants, AAV10, AAV11, AAV12, AAV13, AAV-Rh10, AAV-Rh74, AAV-2i8, AAV-DJ, LK03, NP59, or KP1, or any other AAV. In some cases, the AAV rep protein is from AAV2. In some cases, the AAV capsid is from AAV2, AAV6, AAV7, AVV8, or AAV9. In some cases, the second sequence comprises a promoter, a post-transcriptional regulatory element, or a poly(A). In some cases, the promoter is a CMV promoter. In some cases, the post-transcriptional regulatory element is WPRE. The first plasmid can be constructed to provide, as operatively linked components, a first sequence comprising a promoter, a non-AUG start codon, a rep protein coding sequence, a capsid coding sequence, and a poly(A) sequence; and a second sequence comprising a 5′ ITR, a promoter, a GOI coding sequence, a WPRE, a poly(A) sequence, and a 3′ ITR. In some cases, the promoter in the first sequence is a p5 promoter. In some cases, the promoter in the second sequence is a CMV promoter. In some cases, the non-AUG start codon is ACG.

In some cases, the first plasmid comprises a nucleic acid sequence with about 70%, about 75%, about 80%, about 85%, about 90%, about 95%, about 96%, about 97%, about 98%, about 99%, about 99.5%, or about 99.9% sequence identity to SEQ ID NO: 11. In some cases, the first plasmid comprises a nucleic acid sequence with at least 70%, at least 75%, at least 80%, at least 85%, at least 90%, at least 95%, at least 96%, at least 97%, at least 98%, at least 99%, at least 99.5%, or at least 99.9% sequence identity to SEQ ID NO: 11. In some cases, the first plasmid comprises a sequence of SEQ ID NO: 11.

The GOI can be flanked by two ITRs. In some cases, the exogenous gene flanked by ITRs comprise one or more genes encoding one or more polypeptides, including one or more polypeptides for therapeutic use. In some cases, the one or more polypeptides for therapeutic use include a protein, an antibody, an intrabody, a contractile protein, an enzyme, a hormonal protein, a structural protein, a storage protein, a small molecule, a protein channel, or a transport protein. Non-limiting examples of enzymes include hydrolase, isomerases, nucleases, ligases, transferases, and oxidoreductases. In some cases, the one or more exogenous genes flanked by ITRs encodes a human growth hormone (hGH) gene. In some cases, the one or more exogenous genes encode one or more nucleic acid sequences, including one or more modulatory nucleic acid sequences. In some cases, the one or more modulatory nucleic acid sequences can be of therapeutic use, including anti-sense oligonucleotides, siRNA and miRNA. In some cases, the second plasmid further comprise transcriptional and posttranscriptional regulatory elements. In some cases, the transcriptional and posttranscriptional regulatory elements can modulate (enhance or attenuate) expression of genes delivered by viral vectors. In some cases, the transcriptional and posttranscriptional regulatory elements include a 5′UTR, a promoter (e.g., a CMV promoter), a 3′ UTR, and a poly(A) coding sequence. In some cases, the transcriptional and posttranscriptional regulatory elements further comprise a stuff DNA (Stuffer), and a Woodchuck Hepatitis Virus posttranscriptional regulatory element (WPRE).

In some cases, the first plasmid comprises or consists of a sequence with about 70%, about 75%, about 80%, about 85%, about 90%, about 95%, about 96%, about 97%, about 98%, about 99%, about 99.5%, or about 99.9% sequence identity to SEQ ID NO: 4. In some cases, the first plasmid comprises or consists of a sequence with at least 70%, at least 75%, at least 80%, at least 85%, at least 90%, at least 95%, at least 96%, at least 97%, at least 98%, at least 99%, at least 99.5%, or at least 99.9% sequence identity to SEQ ID NO: 4. In some cases, the first plasmid comprises or consists of a sequence of SEQ ID NO: 4.

The second plasmid can comprise at least one helper element. The helper element can be any element having the helper function for rAAV packaging. In some cases, the helper element is VA gene, E2A(DBP) gene, or E4 gene. In some cases, the helper element is from Ad2 or Ad5. In some cases, the second plasmid comprises a nucleic acid sequence with about 70%, about 75%, about 80%, about 85%, about 90%, about 95%, about 96%, about 97%, about 98%, about 99%, about 99.5%, or about 99.9% sequence identity to SEQ ID NO: 7. In some cases, the second plasmid comprises a nucleic acid sequence with at least 70%, at least 75%, at least 80%, at least 85%, at least 90%, at least 95%, at least 96%, at least 97%, at least 98%, at least 99%, at least 99.5%, or at least 99.9% sequence identity to SEQ ID NO: 7. In some cases, the second plasmid comprises a sequence of SEQ ID NO: 7.

Disclosed herein is a composition comprising two plasmids. The first plasmid can comprise a) a first sequence comprising a sequence encoding an adeno-associated virus (AAV) rep protein and a sequence encoding an AAV capsid, wherein the isolated nucleic acid molecule lacks a promoter upstream of the sequence encoding the AAV rep protein; and b) a second sequence comprising an exogenous gene flanked by inverted terminal repeat (ITR) sequences. In some cases, the first sequence comprises a promoter downstream of the sequence encoding the capsid. In some cases, the first sequence comprises a poly(A) sequence is operably linked to the 3′ end of the sequence encoding the capsid. In some cases, the promoter downstream of the sequence encoding the capsid is operably linked to the 3′ end of the poly(A) sequence. In some cases, the promoter downstream of the sequence encoding the capsid is a p5 promoter. The capsid and the rep protein can be respectively from AAV1, AAV2, AAV2 variants, AAV3 (including AAV 3A and 3B), AAV4, AAV5, AAV6, AAV7, AAV8, AAV9, AAV9 variants, AAV10, AAV11, AAV12, AAV13, AAV-Rh 10, AAV-Rh74, AAV-2i8, AAV-DJ, LK03, NP59, or KP1, or any other AAV. In some cases, the AAV rep protein is from AAV2. In some cases, the AAV capsid is from AAV2, AAV6, AAV7, AAV8, or AAV9. The first plasmid can be constructed to provide, as operatively linked components, a first sequence comprising a rep protein coding sequence, a capsid coding sequence, a poly(A) sequence, and a promoter, wherein the first sequence lacks a promoter at the 5′ end of the rep protein coding sequence; and a second sequence comprising a 5′ ITR, a promoter, a GOI coding sequence, a WPRE, a poly(A) sequence, and a 3′ ITR. In some cases, the promoter in the first sequence is a p5 promoter. In some cases, the promoter in the second sequence is a CMV promoter.

In some cases, the first plasmid comprises a nucleic acid sequence with about 70%, about 75%, about 80%, about 85%, about 90%, about 95%, about 96%, about 97%, about 98%, about 99%, about 99.5%, or about 99.9% sequence identity to SEQ ID NO: 12. In some cases, the first plasmid comprises a nucleic acid sequence with at least 70%, at least 75%, at least 80%, at least 85%, at least 90%, at least 95%, at least 96%, at least 97%, at least 98%, at least 99%, at least 99.5%, or at least 99.9% sequence identity to SEQ ID NO: 12. In some cases, the first plasmid comprises a sequence of SEQ ID NO: 12.

The GOI can be flanked by two ITRs. In some cases, the exogenous gene flanked by ITRs comprise one or more genes encoding one or more polypeptides, including one or more polypeptides for therapeutic use. In some cases, the one or more polypeptides for therapeutic use include a protein, an antibody, an intrabody, a contractile protein, an enzyme, a hormonal protein, a structural protein, a storage protein, a small molecule, a protein channel, or a transport protein. Non-limiting examples of enzymes include hydrolase, isomerases, nucleases, ligases, transferases, and oxidoreductases. In some cases, the one or more exogenous genes flanked by ITRs encodes a human growth hormone (hGH) gene. In some cases, the hGH gene comprises or consists of a sequence of SEQ ID NO: 6. In some cases, the hGH gene comprises or consists of a sequence with about 70%, about 75%, about 80%, about 85%, about 90%, about 95%, or about 99% of sequence identity to SEQ ID NO: 6. In some cases, the hGH gene comprises or consists of a sequence with at least 70%, at least 75%, at least 80%, at least 85%, at least 90%, at least 95%, or at least 99% of sequence identity to SEQ ID NO: 6. In some cases, the one or more exogenous genes encode one or more nucleic acid sequences, including one or more modulatory nucleic acid sequences. In some cases, the one or more modulatory nucleic acid sequences can be of therapeutic use, including anti-sense oligonucleotides, siRNA and miRNA. In some cases, the second plasmid further comprise transcriptional and posttranscriptional regulatory elements. In some cases, the transcriptional and posttranscriptional regulatory elements can modulate (enhance or attenuate) expression of genes delivered by viral vectors. In some cases, the transcriptional and posttranscriptional regulatory elements include a 5′UTR, a promoter (e.g., a CMV promoter), a 3′ UTR, and a poly(A) coding sequence. In some cases, the transcriptional and posttranscriptional regulatory elements further comprise a stuff DNA (Stuffer), and a Woodchuck Hepatitis Virus posttranscriptional regulatory element (WPRE).

In some cases, the first plasmid comprises or consists of a sequence with about 70%, about 75%, about 80%, about 85%, about 90%, about 95%, about 96%, about 97%, about 98%, about 99%, about 99.5%, or about 99.9% sequence identity to SEQ ID NO: 5. In some cases, the first plasmid comprises or consists of a sequence with at least 70%, at least 75%, at least 80%, at least 85%, at least 90%, at least 95%, at least 96%, at least 97%, at least 98%, at least 99%, at least 99.5%, or at least 99.9% sequence identity to SEQ ID NO: 5. In some cases, the first plasmid comprises or consists of a sequence of SEQ ID NO: 5.

The second plasmid can comprise at least one helper element. The helper element can be any element having the helper function for rAAV packaging. In some cases, the helper element is VA gene, E2A(DBP) gene, or E4 gene. In some cases, the helper element is from Ad2 or Ad5. In some cases, the second plasmid comprises a nucleic acid sequence with about 70%, about 75%, about 80%, about 85%, about 90%, about 95%, about 96%, about 97%, about 98%, about 99%, about 99.5%, or about 99.9% sequence identity to SEQ ID NO: 7. In some cases, the second plasmid comprises a nucleic acid sequence with at least 70%, at least 75%, at least 80%, at least 85%, at least 90%, at least 95%, at least 96%, at least 97%, at least 98%, at least 99%, at least 99.5%, or at least 99.9% sequence identity to SEQ ID NO: 7. In some cases, the second plasmid comprises a sequence of SEQ ID NO: 7.

The compositions disclosed herein provide several advantages for producing rAAV particles. Without being limited to any specific theory, the disclosed composition can provide better full/empty ratio as compared to a three-plasmid system by using one plasmid comprising both rAAV proteins (i.e., rep and capsid) and the ITR-flanked exogenous gene where each copy of the exogenous gene expressed would have at least one capsid and thus to avoid empty particles. A three-plasmid system has three plasmids, one comprising the GOI, the other comprising the coding sequences for rep and capsid, and the third one is the helper plasmid to provide helper functions. One drawback with three plasmid systems is a cell could receive only the rep and capsid genes or the rep, capsid, and helper genes. These cells would still produce assembly AAV capsids but would not be able to package the GOI into the capsids. During rAAV manufacturing process, empty particles that lack the transgene are produced and therefore unable to provide the intended therapeutic benefits. These empty particles can be considered a product-related impurity that can raise immunogenicity and efficacy concerns. (Amanda K. Werle, et al., Molecular Therapy—Methods & Clinical Development, 2021 (23), 254-262). Thus, there can be a benefit to control the empty particles and increase the full/empty ratio for rAAV production.

The compositions disclosed herein can also reduce homologous recombination caused by AAV rep or capsid genes. Without being limited to any specific theory, the composition disclosed herein can reduce homologous recombination by separating the coding sequence of rep protein and the coding sequence of capsid into two different vectors and thus increase the full/empty ratio.

In some cases, the compositions disclosed herein can provide rAAV particles, prior to any purification steps, with a full/empty ratio of about 5%, about 10%, about 15%, about 20%, about 25%, about 30%, about 35%, about 40%, about 45%, about 50%. In some cases, the compositions disclosed herein can provide rAAV particles, prior to any purification steps, with a full/empty ratio at least 5%, at least 10%, at least 15%, at least 20%, at least 25%, at least 25%, at least 30%, at least 35%, at least 40%, at least 45%, at least 50%. In some cases, the compositions disclosed herein can provide rAAV particles, prior to any purification steps, with a full/empty ratio from about 5% to about 50%, from about 5% to about 45%, from about 5% to about 40%, from about 5% to about 35%, from about 5% to about 30%, from about 5% to about 25%, from about 5% to about 20%, from about 5% to about 15%, from about 5% to about 10%, from about 10% to about 50%, from about 10% to about 45%, from about 10% to about 40%, from about 10% to about 35%, from about 10% to about 30%, from about 10% to about 25%, from about 10% to about 20%, from 10% to about 15%, from about 15% to about 50%, from about 15% to about 45%, from about 15% to about 40%, from about 15% to about 35%, from about 15% to about 30%, from about 15% to about 25%, from about 15% to about 20%, from about 20% to about 50%, from about 20% to about 45%, from about 20% to about 40%, from about 20% to about 35%, from about 20% to about 30%, from about 20% to about 25%, from about 25% to about 50%, from about 25% to about 45%, from about 25% to about 40%, from about 25% to about 35%, from about 25% to about 30%, from about 30% to about 50%, from about 30% to about 45%, from about 30% to about 40%, from about 30% to about 35%, from about 35% to about 50%, from about 35% to about 45%, from about 35% to about 40%, from about 40% to about 50%, from 40% to about 45%, or from about 45% to about 50%. Table 1 provides the components of the plasmids disclosed herein.

TABLE 1
Plasmids and Components

| | | Helper | |
|---|---|---|---|
| Plasmid # | GOI Component | Component | RepCap Component |
| Plasmid # 11 | N/A | VA-E2A-E4 | p5-ACG-Rep2-Cap2-pA |
| Plasmid # 12 | N/A | VA-E2A-E4 | p5-ACG-Rep2-pA |
| Plasmid # 13 | N/A | VA-E2A-E4 | Rep2-Cap2-pA-p5 |
| Plasmid # 14 | ITR-CMV-hGH- | N/A | Ef1α-Cap2-pA |
| | WPRE-pA-ITR | | |
| Plasmid # 15 | ITR-CMV-hGH- | N/A | p5-ACG-Rep2-Cap2-pA |
| | WPRE-pA-ITR | | |
| Plasmid # 16 | ITR-CMV-hGH- | N/A | Rep2-Cap2-pA-p5 |
| | WPRE-pA-ITR | | |
| Plasmid #17 | ITR-CMV-hGH- | N/A | p40-Cap2-pA |
| | WPRE-pA-ITR | | |
| Plasmid #05 | N/A | N/A | Rep2-Cap2-pA-p5 |
| Plasmid #03 | N/A | N/A | p5-ACG-Rep2-Cap2-pA |
| Plasmid #02 | ITR-CMV-hGH- | N/A | N/A |
| | WPRE-pA-ITR | | |

### Methods of Production

The AAV expression systems provided herein can avoid using helper viruses and can include helper plasmids comprising genes from helper viruses. Exemplary host cells include HEK293 cells, HEK293T cells, human fibrosarcoma (HT-1080) cells, differentiated hepatocyte-derived carcinoma (Huh-7) cells, PER.C6 cells, COS cells, murine myeloma (NS0) cells, HeLa cells, Baby Hamster Kidney (BHK) cells, KB cells, and other mammalian cell lines. An rAAV particle can also be produced by using insect cells that are susceptible to baculovirus infection, such as High Five, Sf9, Se301, SeIZD2109, SeUCR1, Sf9, Sf900+, Sf21, BTI-TN-5B1-4, MG-1, Tn368, HzAm1, BM-N, Ha2302, Hz2E5 and Ao38. Mammalian cells that can be used can include HEK293, HeLa, CHO, NS0, SP2/0, PER.C6, Vero, RD, BHK, HT 1080, A549, Cos-7, ARPE-19 and MRC-5 cells. In some cases, the host cell comprises an E1 gene from the Ad5 genome. In some cases, the host cell is a mammalian cell. In some cases, the mammalian cell is HEK293 cell or its derivatives such as 293T cells.

Baculovirus can be a double-stranded circular DNA virus, belonging to Baculoviridae virus family, and can have a genome size of 90 kb-230 kb.

The present disclosure further provides a recombinant adeno-associated virus (rAAV) particle that can be prepared by introducing or transfecting the composition of the present disclosure into any host cell. In some cases, the introduction or transfection of the composition disclosed herein includes electroporation, calcium phosphate precipitation, liposome-mediated transfection. In some cases, the composition disclosed herein is transfected into 293 cells.

In some cases, the composition of the present disclosure is delivered into an insect cell. In some cases, the method includes electroporation, calcium phosphate precipitation, liposome-mediated transfection, and/or infection. In some cases, the composition is transfected into the insect cell. In some cases, the composition is stably transfected into the insect cell.

In some cases, the rAAV virus particles can be isolated and purified from the cells. For example, the rAAV can be purified using centrifugation, HPLC, hydrophobic interaction chromatography (HIC), anion exchange chromatography, cation exchange chromatography, size exclusion chromatography, ultrafiltration, gel electrophoresis, affinity chromatography, other purification techniques, or any combinations thereof.

In some cases, the rAAV particles produced by using the nucleic acid molecules disclosed herein or the composition disclosed herein, prior to any purification steps, comprise a full/empty ratio of about 5%, about 10%, about 15%, about 20%, about 25%, about 30%, about 35%, about 40%, about 45%, about 50%. In some cases, the compositions disclosed herein can provide rAAV particles, prior to any purification steps, with a full/empty ratio at least 5%, at least 10%, at least 15%, at least 20%, at least 25%, at least 25%, at least 30%, at least 35%, at least 40%, at least 45%, at least 50%. In some cases, the compositions disclosed herein can provide rAAV particles, prior to any purification steps, with a full/empty ratio from about 5% to about 50%, from about 5% to about 45%, from about 5% to about 40%, from about 5% to about 35%, from about 5% to about 30%, from about 5% to about 25%, from about 5% to about 20%, from about 5% to about 15%, from about 5% to about 10%, from about 10% to about 50%, from about 10% to about 45%, from about 10% to about 40%, from about 10% to about 35%, from about 10% to about 30%, from about 10% to about 25%, from about 10% to about 20%, from 10% to about 15%, from about 15% to about 50%, from about 15% to about 45%, from about 15% to about 40%, from about 15% to about 35%, from about 15% to about 30%, from about 15% to about 25%, from about 15% to about 20%, from about 20% to about 50%, from about 20% to about 45%, from about 20% to about 40%, from about 20% to about 35%, from about 20% to about 30%, from about 20% to about 25%, from about 25% to about 50%, from about 25% to about 45%, from about 25% to about 40%, from about 25% to about 35%, from about 25% to about 30%, from about 30% to about 50%, from about 30% to about 45%, from about 30% to about 40%, from about 30% to about 35%, from about 35% to about 50%, from about 35% to about 45%, from about 35% to about 40%, from about 40% to about 50%, from 40% to about 45%, or from about 45% to about 50%.

In some cases, the production yield of the rAAV particles produced by using the nucleic acid molecules disclosed herein or the composition disclosed herein, is higher than that of the rAAV particles produced by using a three-plasmid system. In some cases, the production yield of the rAAV particles produced by using the nucleic acid molecules disclosed herein or the composition disclosed herein, is at least 1.1-fold, 1.2-fold, 1.3-fold, 1.4-fold, 1.5-fold, 1.6-fold, 1.7-fold, 1.8-fold, 1.9-fold, 2.0-fold, 2.1-fold, 2.2-fold, 2.3-fold, 2.4-fold, 2.5-fold, 2.6-fold, 2.7-fold, 2.8-fold, 2.9-fold, 3.0-fold, 3.1-fold, 3.2-fold, 3.3-fold, 3.4-fold, 3.5-fold, 3.6-fold, 3.7-fold, 3.8-fold, 3.9-fold, 4.0-fold, 4.1-fold, 4.2-fold, 4.3-fold, 4.4-fold, 4.5-fold, 4.6-fold, 4.7-fold, 4.8-fold, 4.9-fold, 5.0-fold, 5.1-fold, 5.2-fold, 5.3-fold, 5.4-fold, 5.5-fold, 5.6-fold, 5.7-fold, 5.8-fold, 5.9-fold, 6.0-fold, 6.1-fold, 6.2-fold, 6.3-fold, 6.4-fold, 6.5-fold, 6.6-fold, 6.7-fold, 6.8-fold, 6.9-fold, 7.0-fold, 7.1-fold, 7.2-fold, 7.3-fold, 7.4-fold, 7.5-fold, 7.6-fold, 7.7-fold, 7.8-fold, 7.9-fold, 8.0-fold, 8.1-fold, 8.2-fold, 8.3-fold, 8.4-fold, 8.5-fold, 8.6-fold, 8.7-fold, 8.8-fold, 8.9-fold, 9.0-fold, 9.1-fold, 9.2-fold, 9.3-fold, 9.4-fold, 9.5-fold, 9.6-fold, 9.7-fold, 9.8-fold, 9.9-fold, or 10.0-fold of that of the rAAV particles produced by using a three-plasmid system.

Recombinant Adeno-Associated Virus (rAAV)

The present disclosure also provide recombinant AAVs (rAAVs) produced by using the nucleic acid molecules or compositions disclosed herein. The produced rAAV can comprise an exogenous gene. The exogenous gene can be any gene flanked by inverted terminal repeat (ITR) sequences. The exogenous gene can have a size from about 1 kilobases to about 10 kilobases, from about 1 kilobases to about 9 kilobases, from about 1 kilobases to about 8 kilobases, from about 2 kilobases to about 7 kilobases, from about 3 kilobases to about 6 kilobases, from about 4 kilobases to about 5 kilobases, from about 1 kilobases to about 5 kilobases, from about 2 kilobases to about 4 kilobases, or from about 2 kilobases to about 5 kilobases. In some cases, the exogenous gene has a size of about 1 kilobases, about 2 kilobases, about 3 kilobases, about 4 kilobases, about 5 kilobases, about 6 kilobases, about 7 kilobases, about 8 kilobases, about 9 kilobases, or about 10 kilobases. In some cases, the exogenous gene has a size larger than 1 kilobases, 2 kilobases, 3 kilobases, 4 kilobases, 5 kilobases, 6 kilobases, 7 kilobases, or 8 kilobases. In some cases, the exogenous gene flanked by ITRs comprise one or more genes encoding one or more polypeptides, including one or more polypeptides for therapeutic use. In some cases, the one or more polypeptides for therapeutic use include a protein, an antibody, an intrabody, a contractile protein, an enzyme, a hormonal protein, a structural protein, a storage protein, a small molecule, a protein channel, or a transport protein. Non-limiting examples of enzymes include hydrolase, isomerases, nucleases, ligases, transferases, and oxidoreductases. In some cases, the exogenous genes comprise a gene encoding a peptide or protein that would result in amelioration of symptoms associated with a disease or disorder.

Exemplary exogenous genes can include human growth hormone (hGH) gene, a RPE65 gene, a LPL gene, a CNGA3 gene, a CNGB3 gene, a mitochondrial DNA, a XLRS gene, a FVIII gene, a FIX gene, an AAT gene, a SMN gene, a MicroDMD gene, an α-Sarcoglycan gene, a Follistatin gene, a dysferlin gene, an NF-κB gene, an IFN-β gene, an ARSA gene, a NGF gene, a Neurturin gene, an AADC gene, a TPP1 gene, a SGSH gene, a SUMF1 gene, and/or an OTC gene. The rAAV can comprise any rep protein disclosed herein. The rAAV can comprise any capsid disclosed herein. In some cases, the rAAV comprises a capsid and a rep protein from the same AAV serotype. In some cases, the rAAV comprises a capsid and a rep protein from different AAV serotype.

### Pharmaceutical Composition

In one aspect, provided herein is a pharmaceutical composition comprising the described rAAV particle or the described composition. In some cases, the pharmaceutical composition comprises the rAAV particles of the present disclosure and a pharmaceutically acceptable carrier or excipient.

The type of carrier used in the pharmaceutical formulation can depend on the method of administration of the therapeutic compound. Many methods of preparing pharmaceutical compositions for multiple routes of administration are well known in the art.

In some cases, the pharmaceutical composition is prepared by dissolving the rAAV virus particles of the present disclosure in a suitable solvent. Suitable solvents include water, saline solutions (e.g., NaCl), buffer solutions (e.g., phosphate-buffered saline (PBS)), or other solvents. In some cases, the viral particle pharmaceutical composition can include a surfactant (e.g., Poloxamer, pluronic acid F68). In some cases, the solvent is sterile. In some cases, the viral particle pharmaceutical composition comprises sodium chloride, sodium phosphate and poloxamer. In some cases, the pharmaceutical composition does not comprise any preservatives.

In some cases, the pharmaceutical composition is a suspension. In some cases, the pharmaceutical composition is a solution.

A pharmaceutical composition described herein can comprise any suitable amount of rAAV particles. In some cases, the pharmaceutical composition comprises 1×10\hat{ }9 to 1×10\hat{ }15 vector genomes (vg) per mL. In some cases, the pharmaceutical composition comprises 1×10\hat{ }10 to 1×10\hat{ }14 vg per mL. In some cases, the pharmaceutical composition comprises 5×10\hat{ }10 to 5×10\hat{ }13 vg per mL. In some cases, the pharmaceutical composition comprises 1×10\hat{ }11 to 1×10\hat{ }13 vg per mL. In some cases, the pharmaceutical composition comprises 0.1 to 500 mL in volume. In some cases, the pharmaceutical composition comprises 0.2 to 50 mL in volume. In some cases, the pharmaceutical composition comprises 0.1 to 10 mL in volume.

The pharmaceutical composition disclosed herein can also be combined with other therapeutic agents.

### Kits

Provided herein are kits comprising one or more of the nucleic acid molecules or compositions disclosed herein. The kits can comprise instructions.

In another aspect, the present disclosure provides kits for treating a disease or a condition in a subject in need thereof, comprising a nucleic acid molecule disclosed herein, a composition disclosed herein, an AAV viral particle disclosed herein, or a pharmaceutical composition disclosed herein, and an instruction manual. In some cases, the instructions indicate the administration of the polynucleotide, the AAV viral particle, or the pharmaceutical composition for the treatment of a disease or a condition in a subject in need thereof.

In some cases, the kit further comprises a container. In some cases, the container is configured to deliver the nucleic acid molecule disclosed herein, the composition disclosed herein, the AAV viral particle disclosed herein, or the pharmaceutical composition. In some cases, the container comprises a vial, a dropper, bottles, tubes, and syringes. In some of cases, the container is a syringe used for administering the nucleic acid molecule disclosed herein, the composition disclosed herein, the AAV viral particle disclosed herein, or the pharmaceutical composition.

In some cases, the kit comprises (i) a first vector comprising a sequence encoding a rep protein and a capsid and an exogenous gene (e.g., Plasmid #15 or #6), and (ii) a second vector comprising one or more helper elements. In some cases, the kit comprises (i) a first vector comprising a sequence encoding a rep protein and a capsid and one or more helper elements (e.g., Plasmid #11 or #3), and (ii) a second vector comprising an exogenous gene. In some cases, the kit comprises (i) a first vector comprising a sequence encoding a capsid and an exogenous gene (e.g., Plasmid #14), and (ii) a second vector comprising a sequence encoding a rep protein and one or more helper elements (e.g., Plasmid #12). In some cases, the kit comprises (i) a first vector comprising a sequence encoding a rep protein and an exogenous gene, and (ii) a second vector comprising a sequence encoding a capsid and one or more helper elements.

### Method of Treatment

The present disclosure also provides a method for treating a disease or a condition in a subject in need thereof, which comprises administering a therapeutically effective amount of the nucleic acids, the compositions, the rAAV, or the pharmaceutical composition of the present disclosure to a subject in need thereof.

The rAAV particles disclosed herein can be used to treat any gene-related disease. Exemplary diseases include, but not limited to, hGH deficiency, LPL deficiency, LCA, CHM, ACHM, LHON, XLRS, hemophilia A, hemophilia B, AAT deficiency, SMA, DMD, LGMD2D, BMDSIBM, dysferlin deficiency, RA, metachromatic leukodystrophy, Alzheimer's disease, Parkinson's disease, AADC deficiency, Batten disease, Sanfilippo A syndrome, blood ammonia accumulation, and/or hepatitis.

In some cases, the rAAV particles is provided in a therapeutically effective amount that achieves the desired biological effect at a medically acceptable level of toxicity. The dosage can vary according to the route of administration and the severity of the disease. The dosage can also be adjusted according to the weight, age, sex, degree of symptoms of each patient to be treated, or any combinations thereof. The precise dosage and route of administration can ultimately be determined by the treating doctor or veterinarian. The dosage can be routinely changed according to the age and weight of the patient and the severity of the condition to be treated.

In some cases, the frequency of administration can be applied at least once a day, including 2, 3, 4, or 5 times a day. In some cases, the treatment can last for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days.

In some cases, a therapeutically effective amount of the composition can be determined based on the total amount of the therapeutic agent (e.g., rAAV comprising a DNA sequence encoding a hGH protein) contained in the pharmaceutical compositions of the present disclosure. As disclosed above, a therapeutically effective amount is sufficient to achieve a meaningful benefit to the subject (e.g., treating, modulating, curing, preventing and/or ameliorating hemophilia A). For example, a therapeutically effective amount can be an amount sufficient to achieve a desired therapeutic and/or prophylactic effect. In some cases, the amount of a therapeutic agent (e.g., rAAV comprising a DNA sequence encoding a hGH protein) administered to a subject in need thereof depends upon the characteristics of the subject. Such characteristics include the condition, disease severity, general health, age, sex and body weight of the subject. In some cases, both objective and subjective assays can optionally be employed to identify optimal dosage ranges.

A therapeutically effective amount is commonly administered in a dosing regimen that may comprise multiple unit doses. For any particular therapeutic protein or therapeutic agent or therapeutic composition, a therapeutically effective amount (and/or an appropriate unit dose within an effective dosing regimen) can vary, for example, depending on route of administration, on combination with other pharmaceutical agents. In some cases, the specific therapeutically effective amount (and/or unit dose) for any particular patient can depend upon a variety of factors including the disorder being treated and the severity of the disorder; the activity of the specific pharmaceutical agent employed; the specific composition employed; the age, body weight, general health, sex and diet of the patient; the time of administration, route of administration, and/or rate of excretion or metabolism of the specific protein employed; the duration of the treatment; etc.

In some cases, the composition is administered by intravascular injection, intravenous injection, or intra-arterial injection.

### EXAMPLES

The following examples are not intended to limit the scope of what the inventors regard as various aspects of the present invention.

### Example 1. Construct Design of Recombinant AAV (rAAV) Vectors

The nucleic acid sequences encoding rep and cap proteins from AAV2 and the nucleic acid sequence encoding helper elements (E4, E2A and VA), along with their corresponding promoters, were synthesized to generate Plasmids #11 and #13 (FIGS. 2A & 2C). The nucleic acid sequence encoding hGH, along with its corresponding promoter, was synthesized to generate plasmid GOI (pGOI, Plasmid #02, FIG. 7).

The nucleic acid sequence encoding rep and cap proteins from AAV2 and the nucleic acid sequence encoding hGH, along with their corresponding promoters, were synthesized to generate Plasmids #15 and #16 (FIGS. 2E & 2F). The nucleic acid sequence comprising the helper elements (Help, including E4, E2A and VA), along with its corresponding promoter, was synthesized to generate plasmid Help (pHelp, Plasmid #20, FIG. 4A).

In Plasmids #11 and #15, an alternative start codon (ACG) was introduced upstream of the nucleic acid sequence encoding rep and cap proteins and downstream of p5. In Plasmids #13 and #16, promoter p5 in the sequence encoding rep and cap proteins was placed downstream of the poly(A) sequence instead of upstream of the sequence encoding rep.

The nucleic acid sequence encoding rep protein from AAV2 and the nucleic acid sequence comprising the helper elements (E4, E2A and VA), along with their corresponding promoters, were synthesized to generate Plasmid #12 (FIG. 2B).

The nucleic acid sequence encoding a cap protein from AAV2 and the nucleic acid sequence encoding hGH, along with their corresponding promoters, were synthesized to generate Plasmid #14 (FIG. 2D) and Plasmid #17 (FIG. 9).

In Plasmid #12, an alternative start codon (ACG) was introduced upstream of the nucleic acid sequence encoding rep protein and downstream of p5. In Plasmid #14, Ef1α promoter was used for transcription of cap protein. In Plasmid #17, p40 promoter was used for transcription of cap protein.

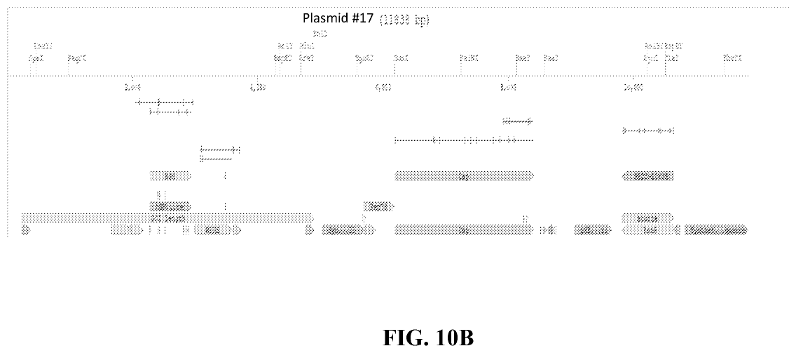

The plasmid maps for the constructs used herein (Plasmids #11-16 and Plasmid #17) are provided in FIGS. 3A-3F and FIGS. 10A-10B. The annotation table for the plasmid map of Plasmid #17 is provided below. The plasmid map of the helper elements (Plasmid #20) is shown in FIG. 4B.

Plasmid #17 Annotation Table

| Annotation Name | Start Position | End Position |
|---|---|---|
| 5′ ITR | 233 | 362 |
| Gene Of Interest | 363 | 4766 |
| 3′ ITR | 4767 | 4896 |
| P40 promoter | 5696 | 5875 |
| Capsid 5′ UTR | 5696 | 6182 |
| Capsid (VP1) Coding | 6199 | 8406 |
| Sequence | | |
| AAV2 PolyA Signal | 8407 | 8431 |

### Example 2. rAAV Particle Production by Two-Plasmid Systems

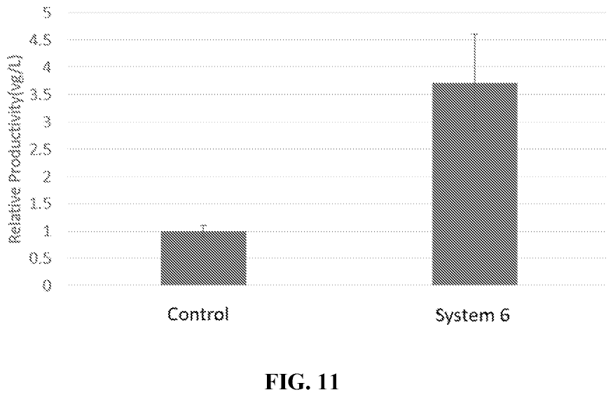

The AAV plasmids were transfected in HEK293 cells for viral packaging. The two-plasmid systems used for transfection are shown in Table 2. The titer of the AAV particles in the culture supernatant for each two-plasmid system was measured by a dPCR-based vector genome tittering assay. A three-plasmid system was used as a control. The control three-plasmid system had three plasmids each encoding the cap and rep, the GOI (hGH), and the helper element, respectively. The controls for this experiment utilized the previously described plasmids, Plasmid #02, Plasmid #05, and Plasmid #20. As shown in FIG. 5 and FIG. 11, all of the two-plasmid systems demonstrated rAAV particle production, with System 5 the highest.

TABLE 2
Two-Plasmid Systems

| Two-Plasmid | First | SEQ | Second | SEQ |
|---|---|---|---|---|
| System | Plasmid | ID NO. | Plasmid | ID NO. |
| System 1 | Plasmid #13 | 1 | pGOI (Plasmid | 6 |
| | | | #002) | |
| System 2 | Plasmid #12 | 2 | Plasmid #14 | 15 |
| System 3 | Plasmid #11 | 3 | pGOI (Plasmid | 6 |
| | | | #002) | |
| System 4 | Plasmid #15 | 4 | pHELP (Plasmid | 7 |
| | | | #020) | |
| System 5 | Plasmid #16 | 5 | pHELP (Plasmid | 7 |
| | | | #020) | |
| System 6 | Plasmid #12 | 2 | Plasmid #17 | 17 |

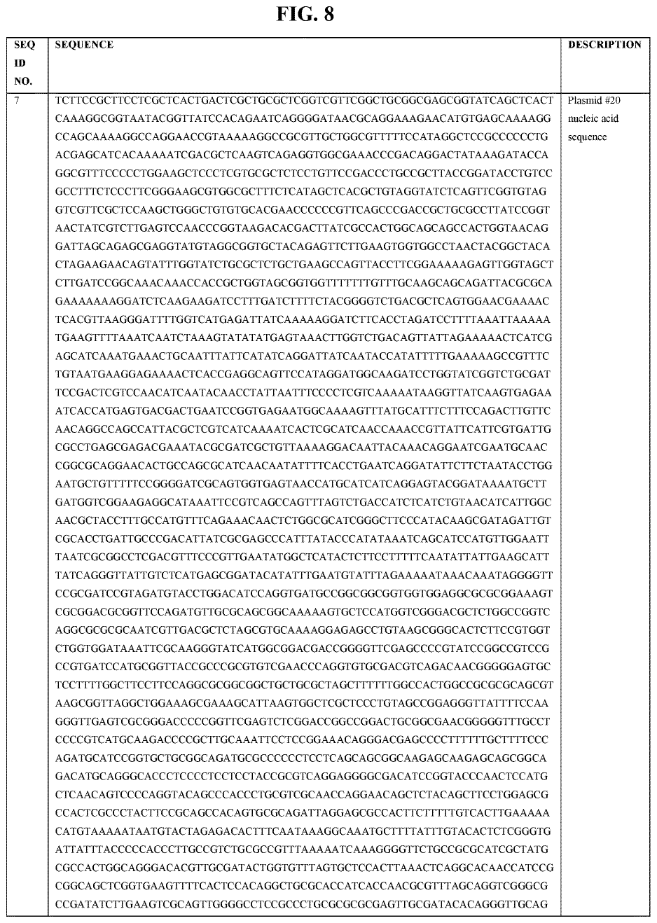

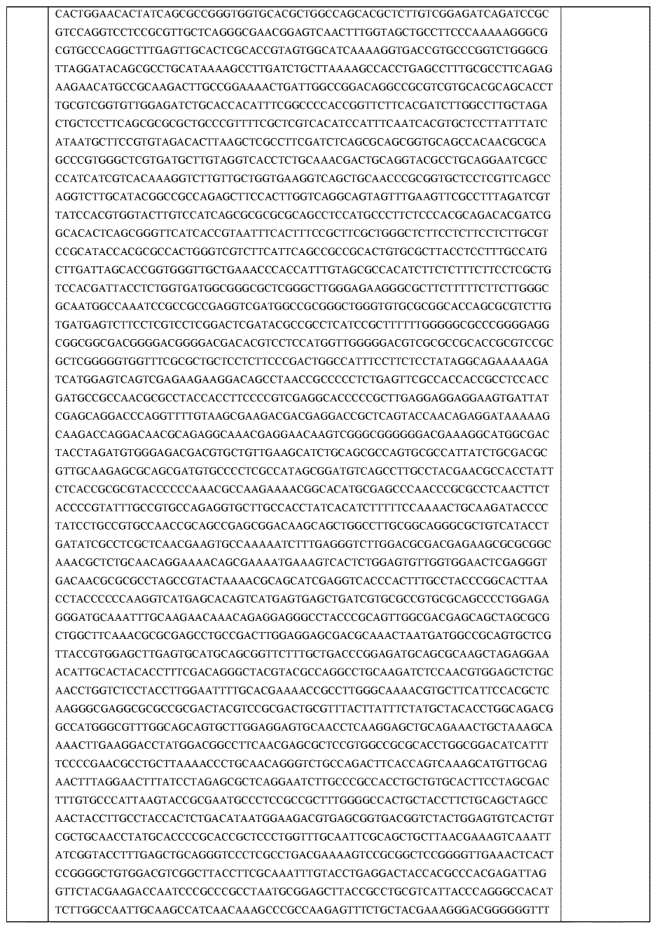

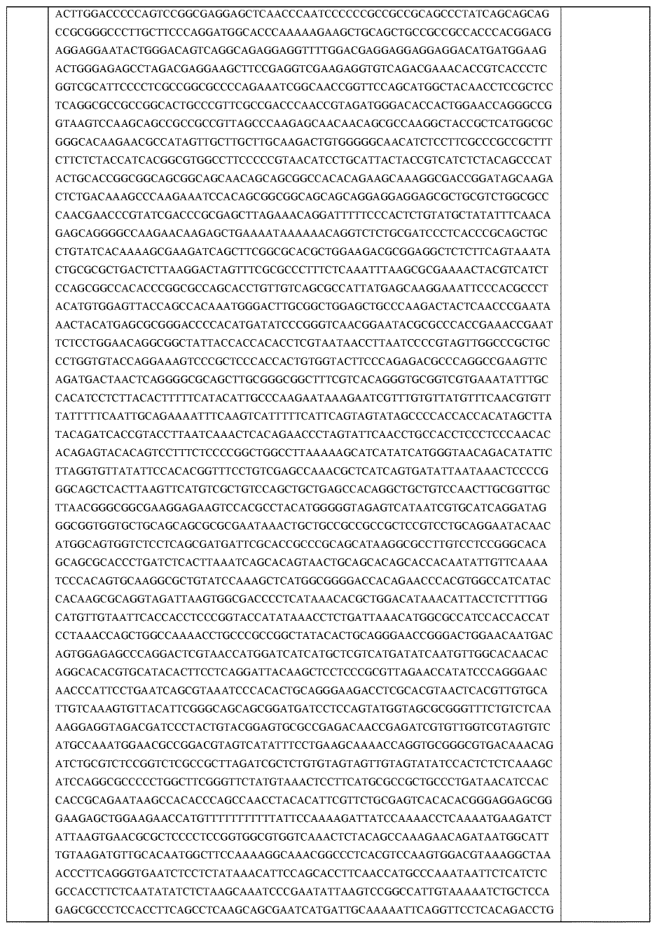

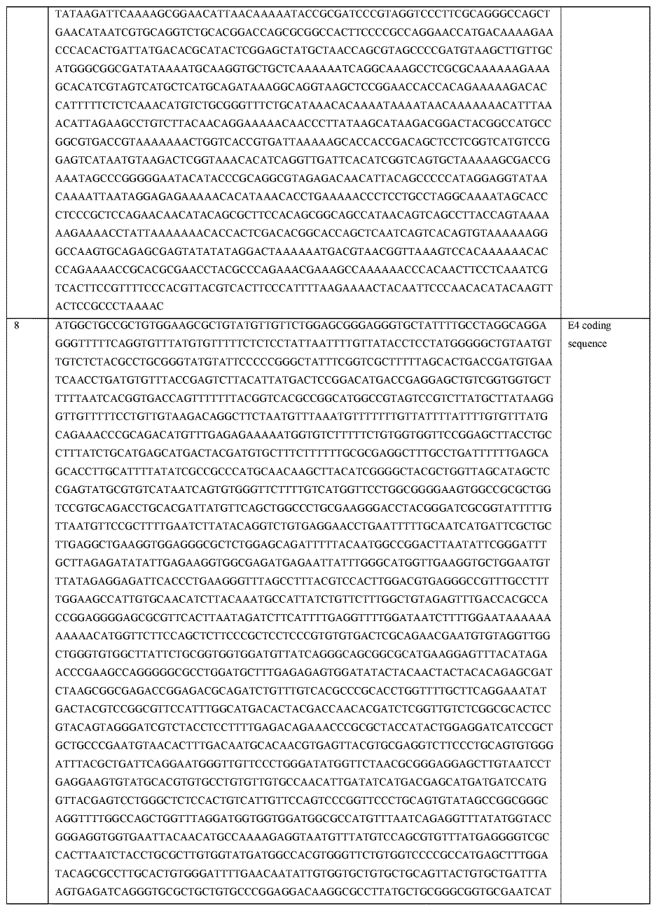

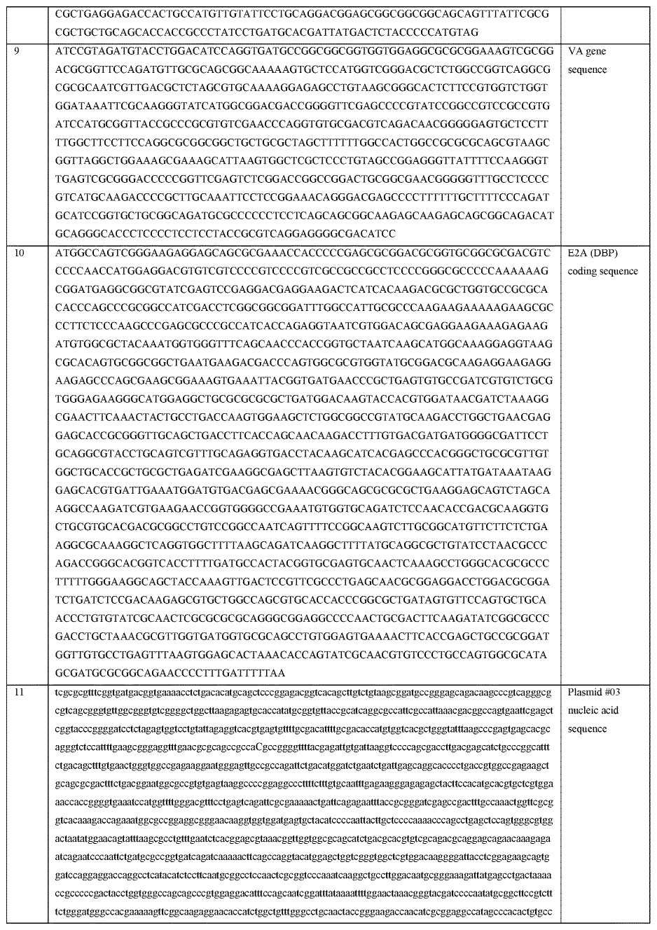

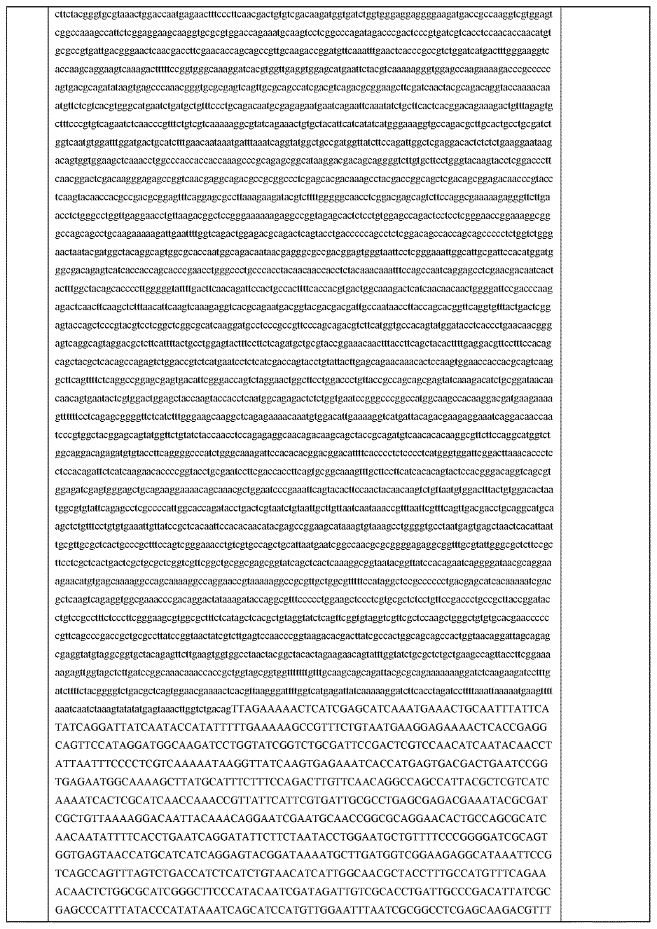

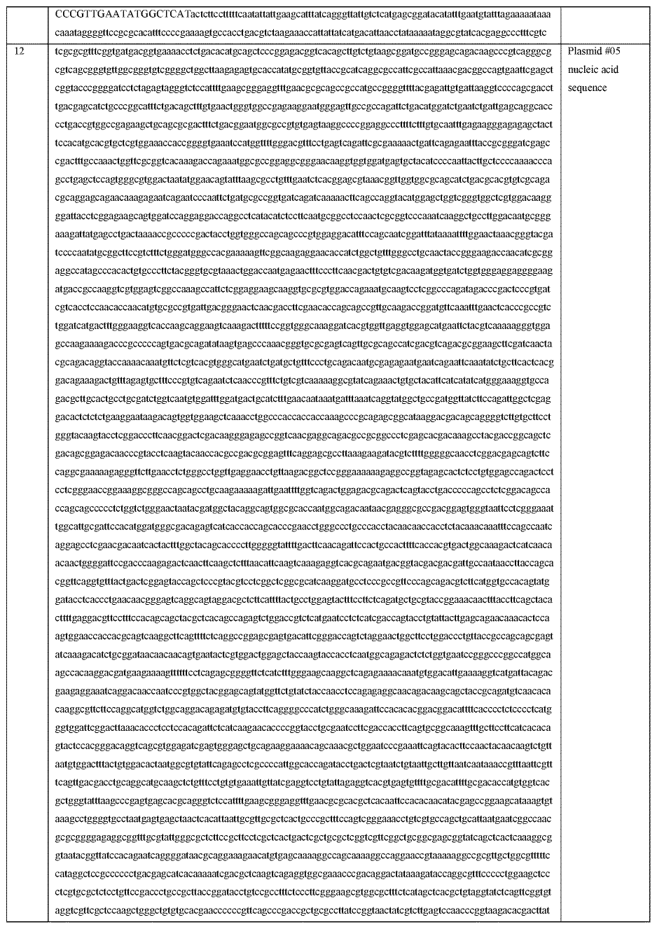

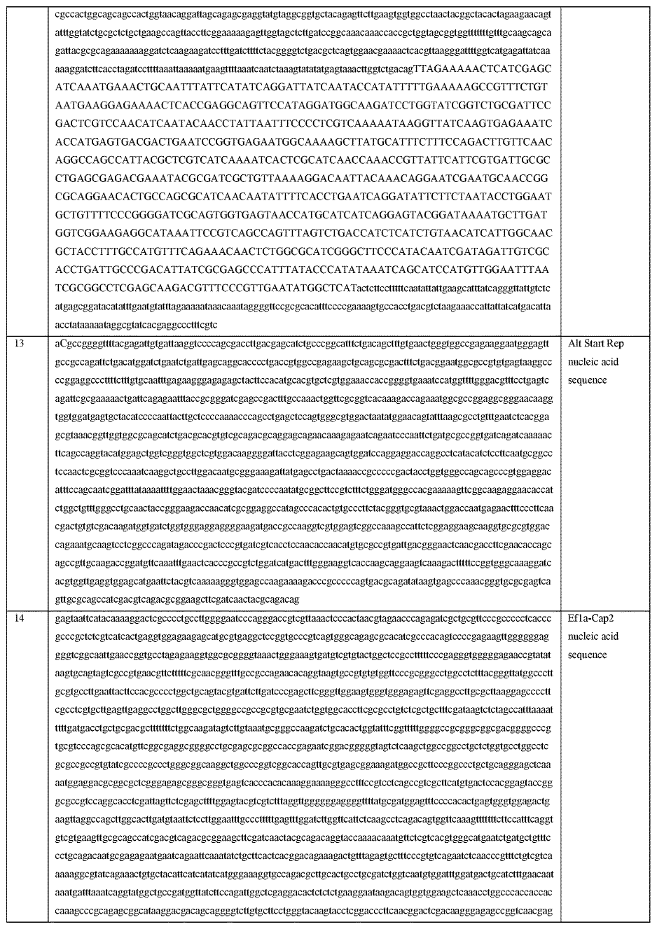

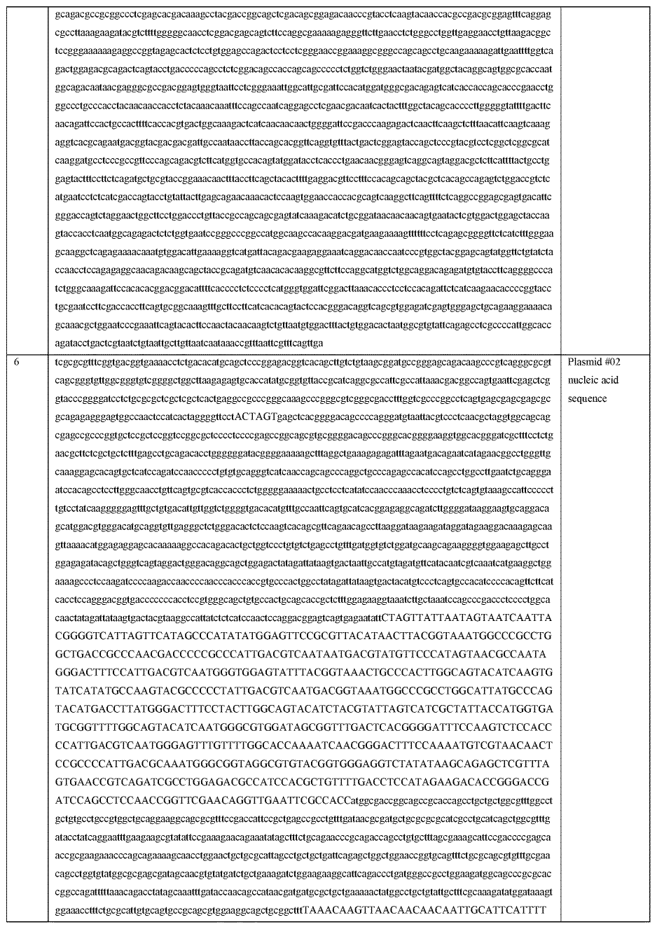

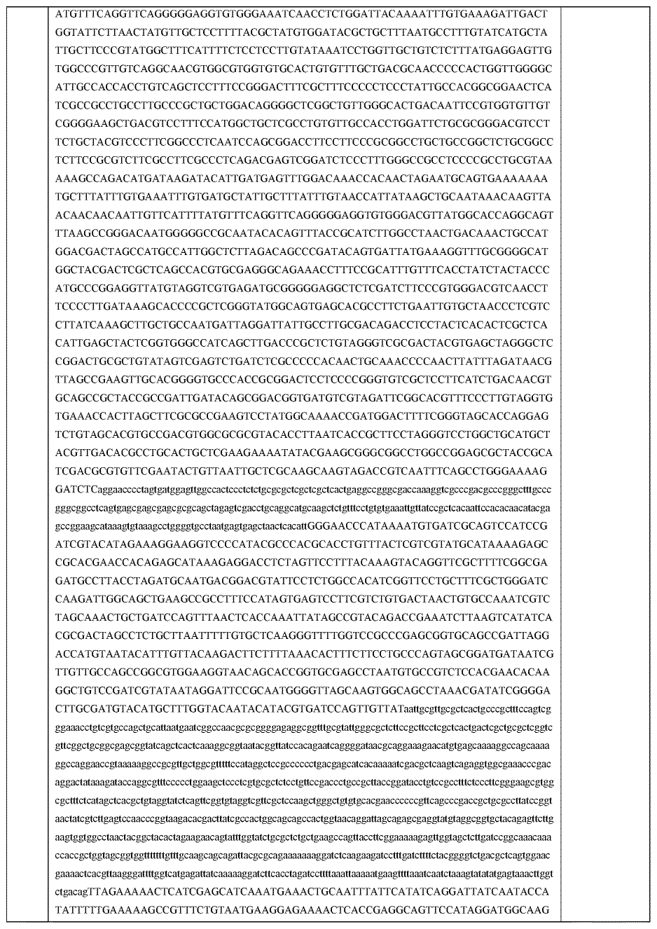

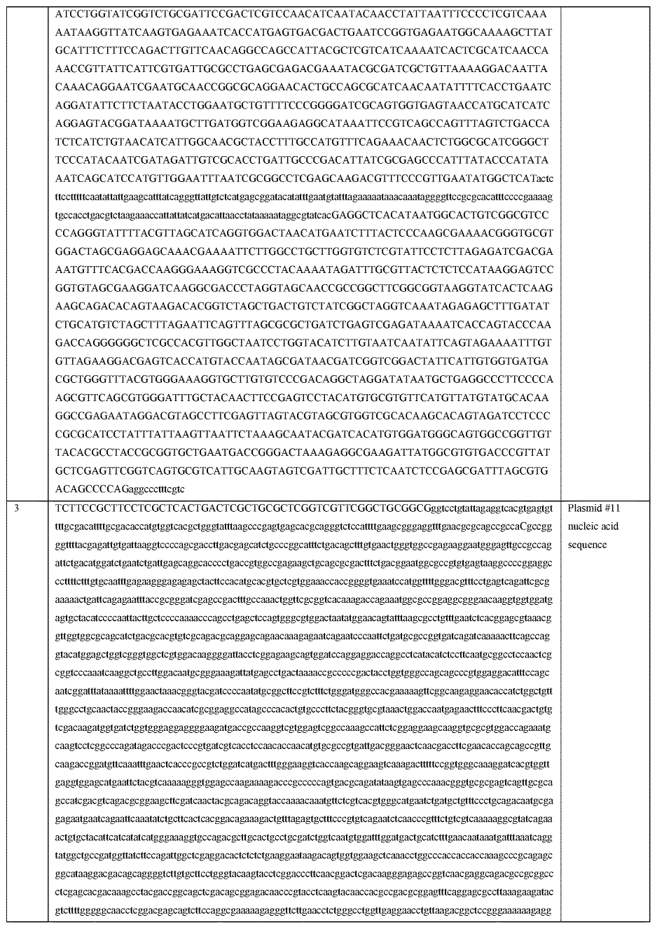

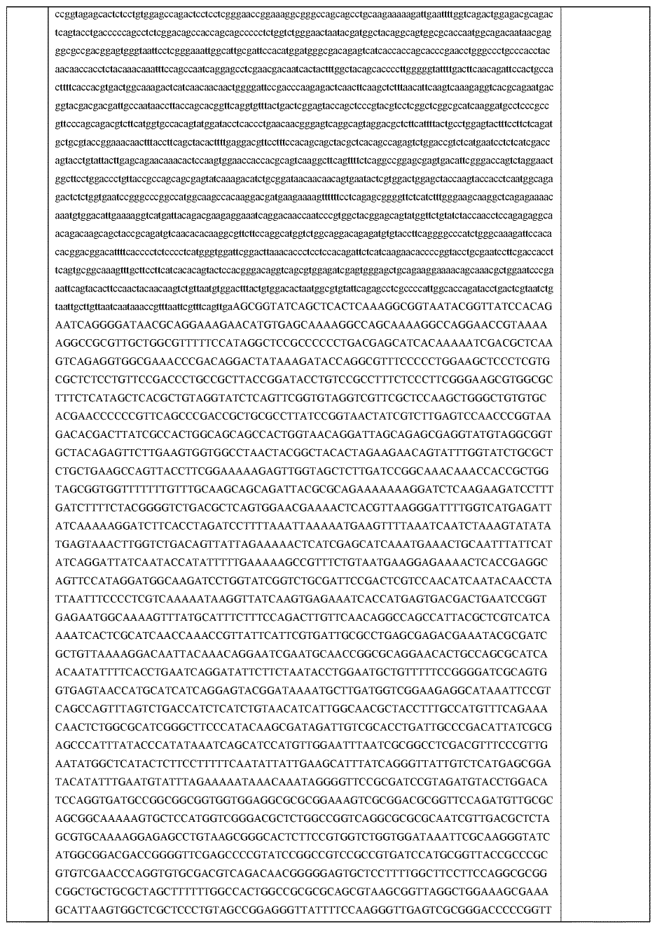

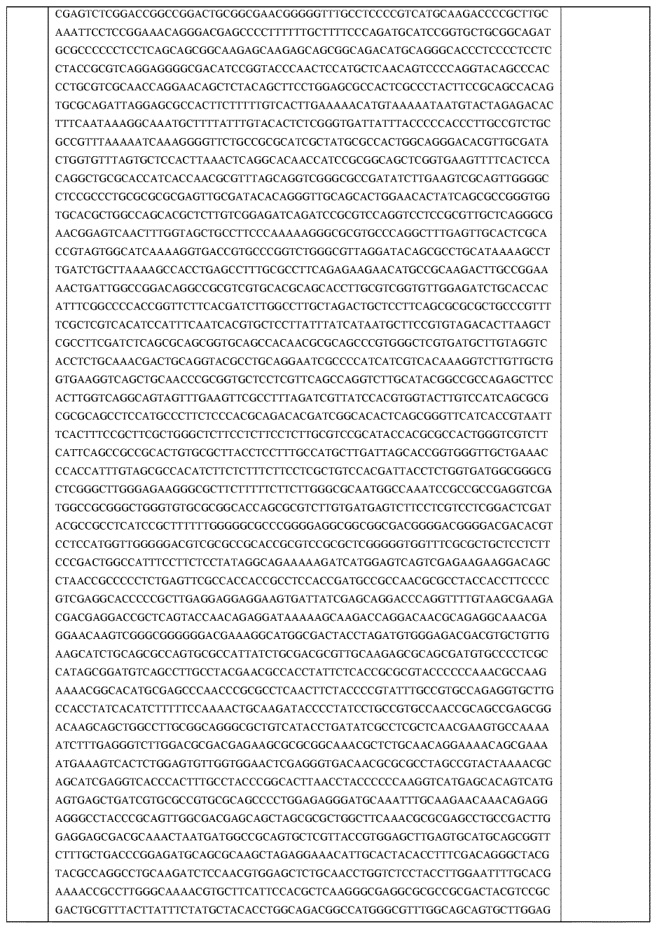

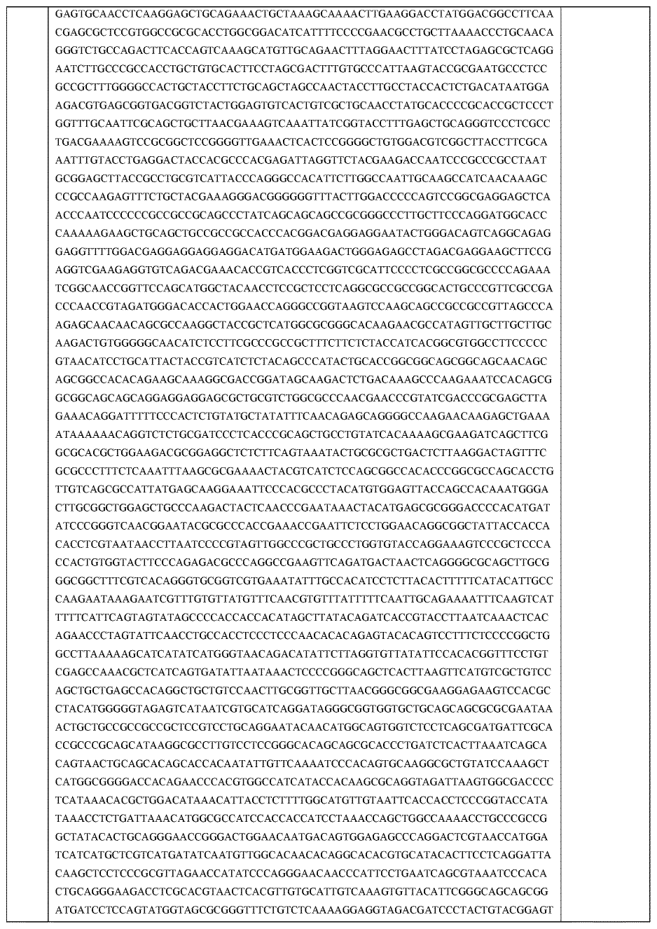

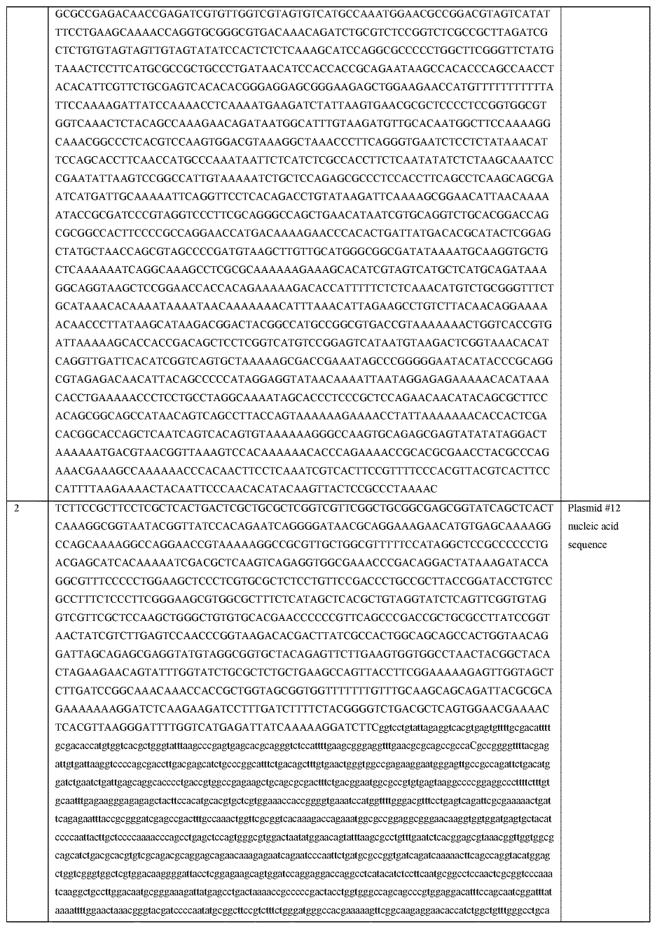

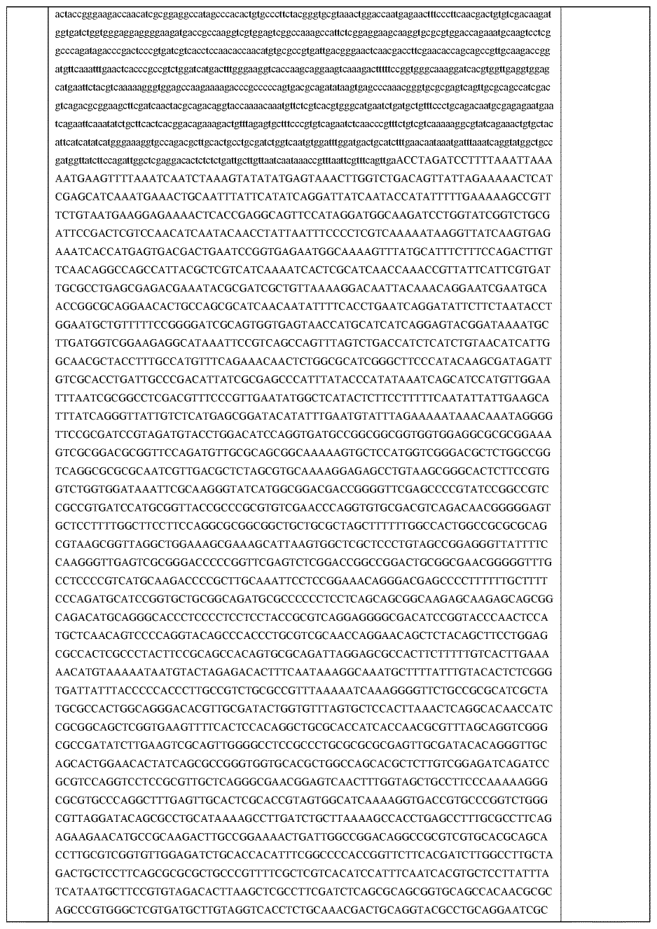

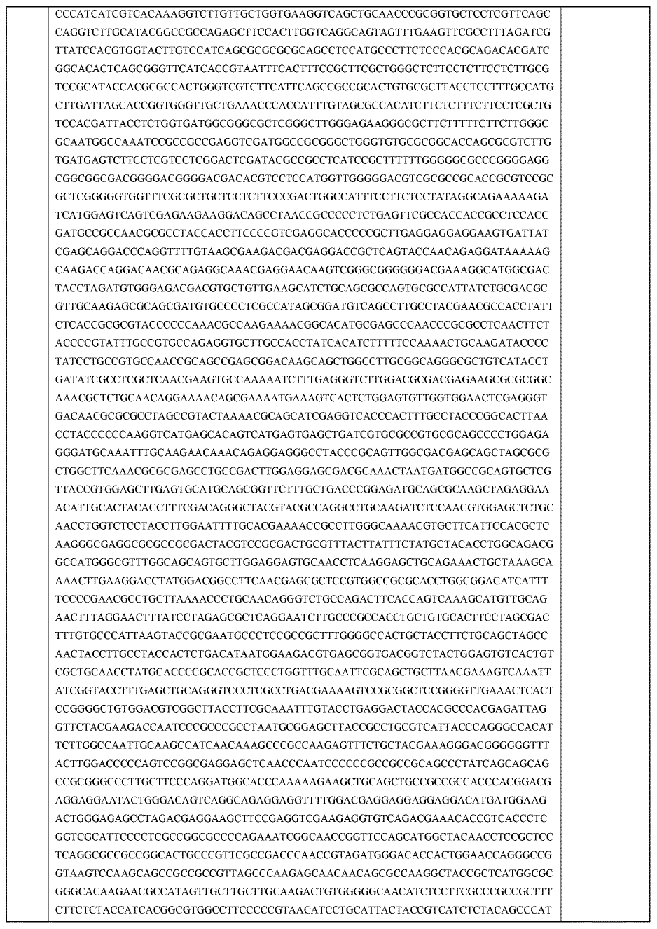

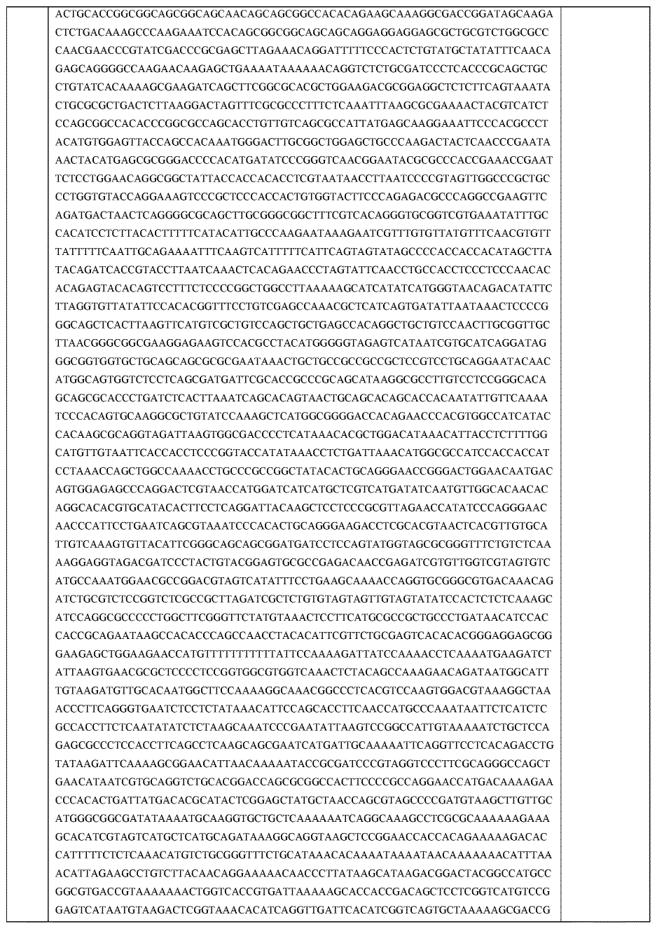

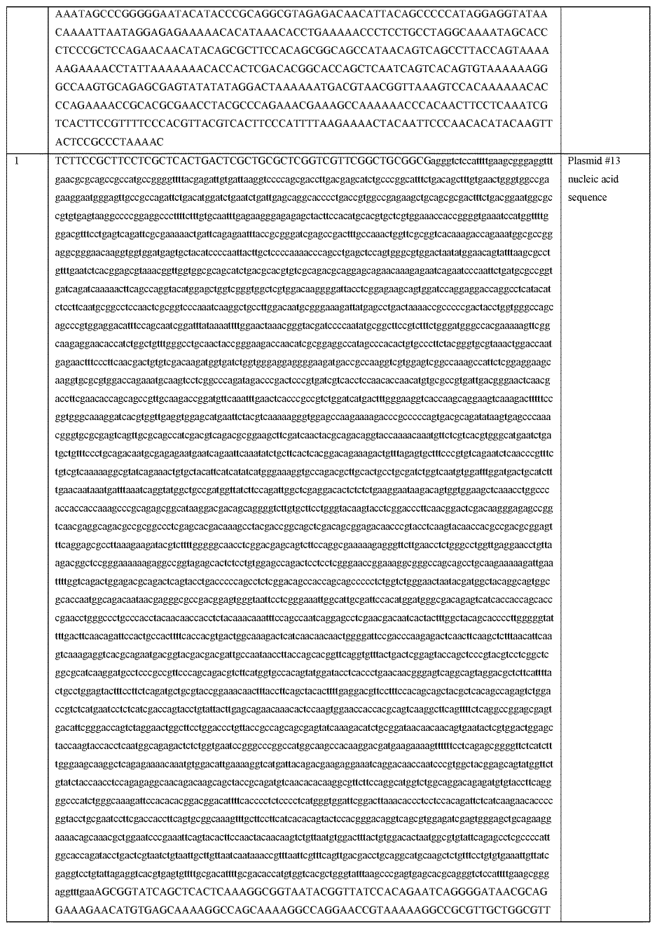

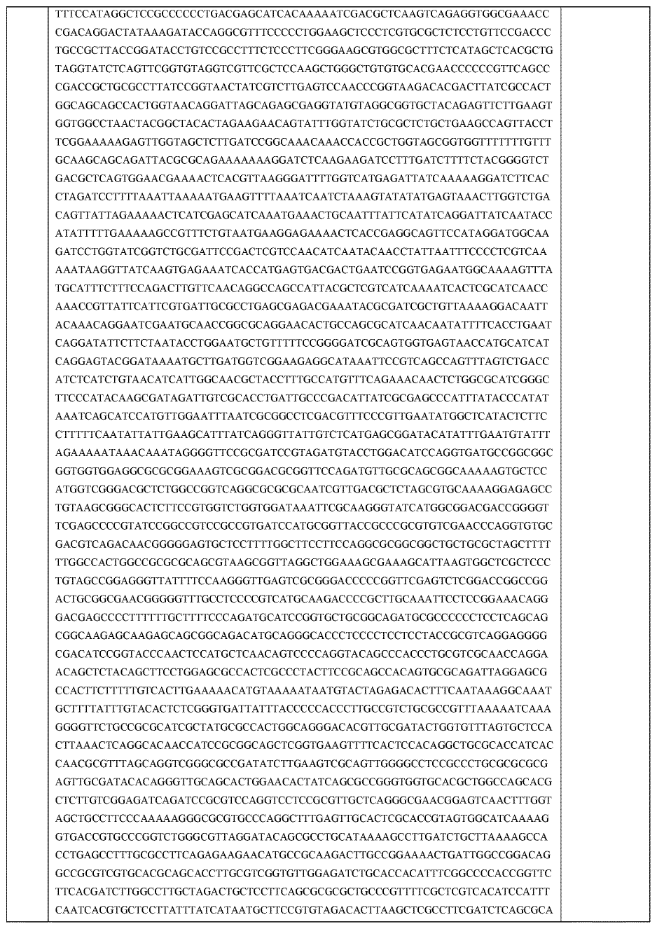

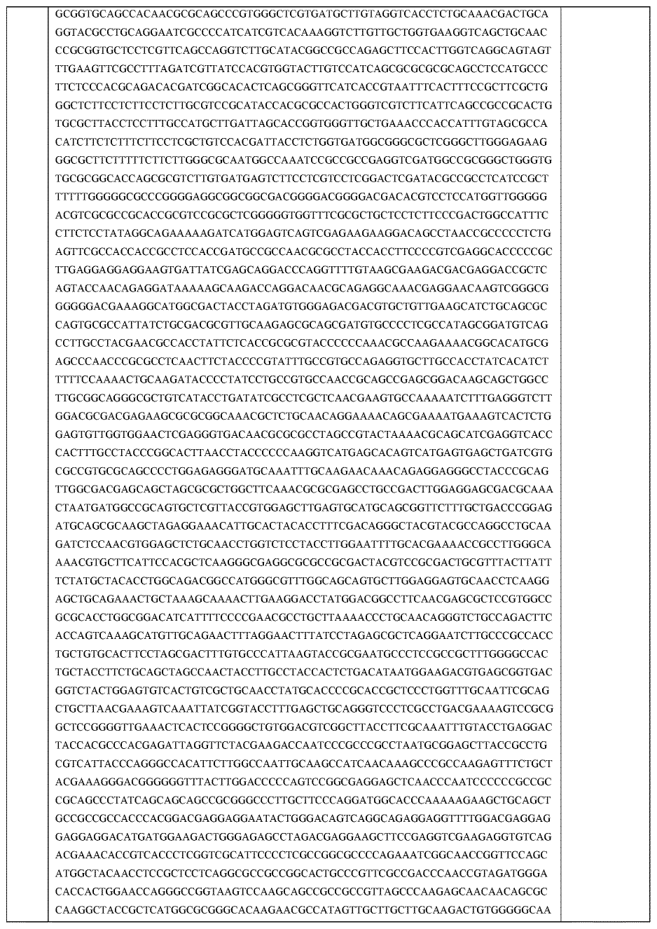

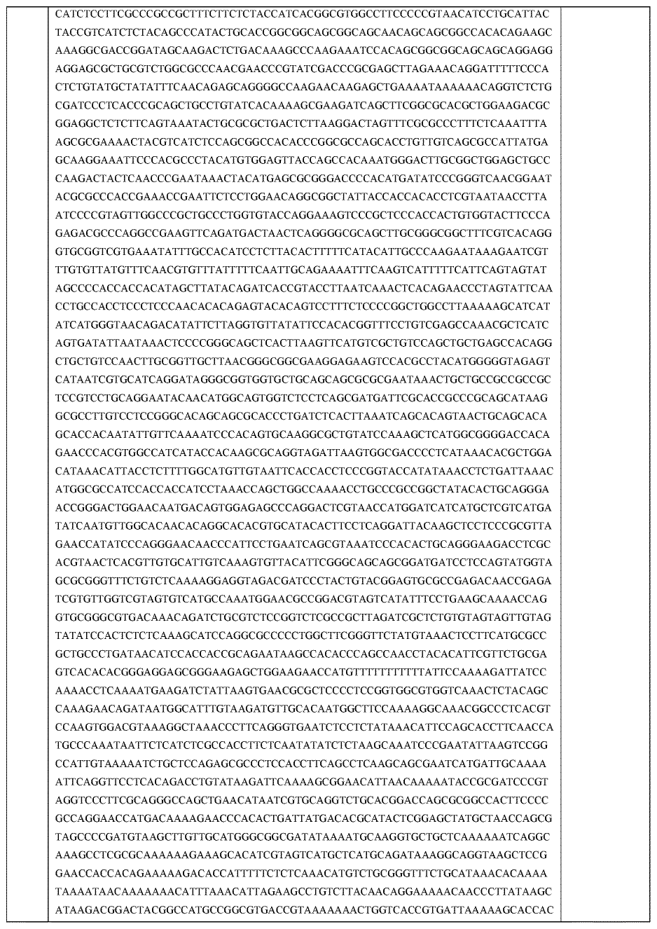

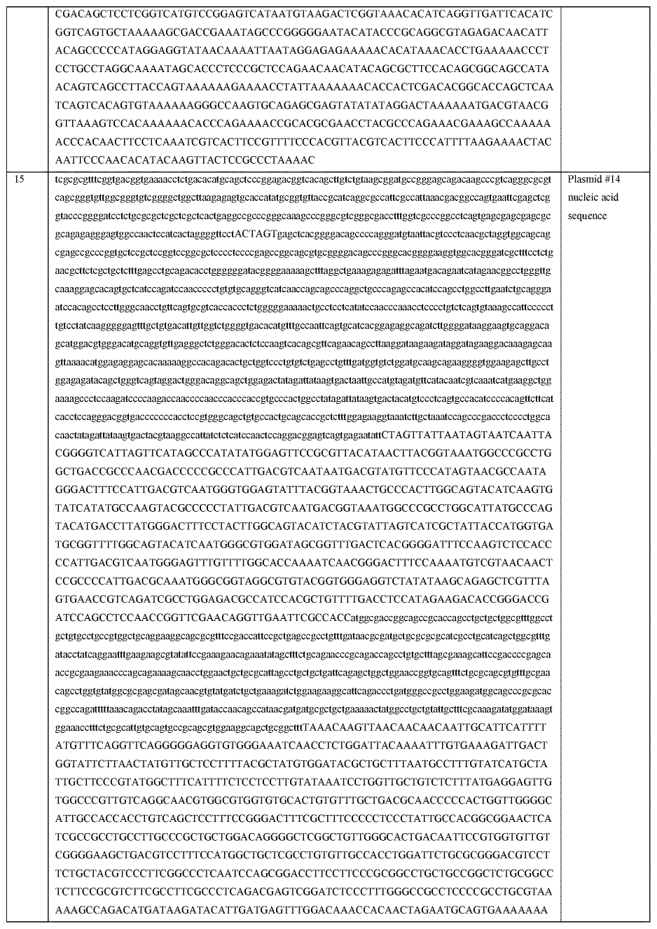

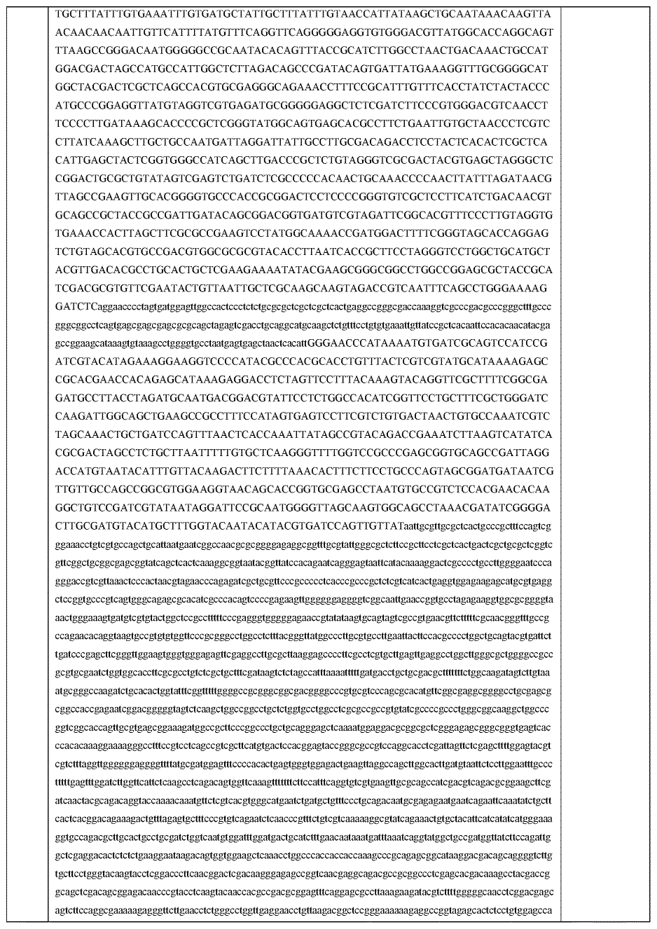

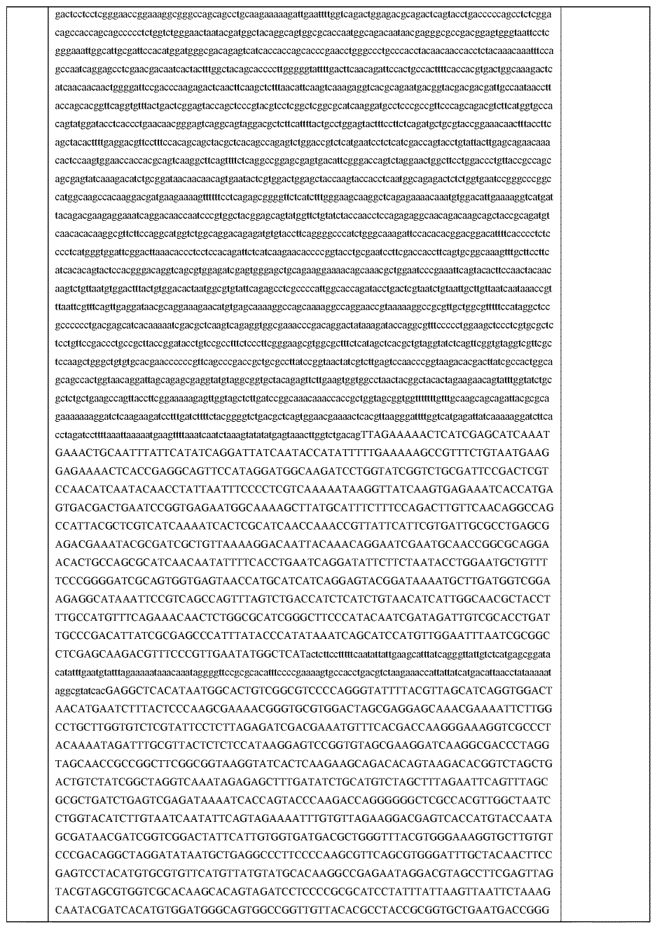

A list of sequences disclosed herein is provided in the table of FIG. 8.

Provided below are exemplary embodiments of the invention:
Embodiment 1: An isolated nucleic acid molecule comprising (a) a sequence encoding an adeno-associated virus (AAV) rep protein or a sequence encoding an AAV capsid, without a p5 promoter upstream of the sequence encoding the AAV rep protein or the sequence encoding an AAV capsid; and (b) an exogenous gene flanked by inverted terminal repeat (ITR) sequences.
Embodiment 2: The isolated nucleic acid molecule of embodiment 1, comprising the sequence encoding the AAV rep protein.
Embodiment 3: The isolated nucleic acid molecule of embodiment 2, wherein the AAV rep protein comprises REP2.
Embodiment 4: The isolated nucleic acid molecule of embodiment 1, comprising the sequence encoding the AAV capsid.
Embodiment 5: The isolated nucleic acid molecule of embodiment 4, wherein the AAV capsid comprises a capsid from AAV1, AAV2, AAV2 variant, AAV3A, AAV3B, AAV4, AAV5, AAV6, AAV7, AAV8, AAV9, AAV9 variant, AAV10, AAV11, AAV12, AAV13, AAV-Rh10, AAV-Rh74, AAV-2i8, AAV-DJ, LK03, NP59, or KP1.
Embodiment 6: The isolated nucleic acid molecule of any one of embodiments 1-5, wherein the exogeneous gene has a size from about 2 kilobases to about 7 kilobases.
Embodiment 7: The isolated nucleic acid molecule of embodiment 1, comprising the sequence encoding the AAV rep protein and the AAV capsid.
Embodiment 8: The isolated nucleic acid molecule of embodiment 7, further comprising a promoter 3′ of the sequence encoding the AAV rep protein and the sequence encoding the AAV capsid.
Embodiment 9: An isolated nucleic acid molecule comprising (a) a sequence encoding a rep AAV protein or a sequence encoding an AAV capsid, without a p5 promoter downstream of the sequence encoding the AAV rep protein or the sequence encoding an AAV capsid; and (b) an exogenous gene flanked by inverted terminal repeat (ITR) sequences.
Embodiment 10: The isolated nucleic acid molecule of embodiment 9, comprising the sequence encoding the AAV rep protein.
Embodiment 11: The isolated nucleic acid molecule of embodiment 10, wherein the AAV rep protein comprises REP2.
Embodiment 12: The isolated nucleic acid molecule of embodiment 9, comprising the sequence encoding the AAV capsid.
Embodiment 13: The isolated nucleic acid molecule of embodiment 12, wherein the AAV capsid comprises a capsid from AAV1, AAV2, AAV2 variant, AAV3A, AAV3B, AAV4, AAV5, AAV6, AAV7, AAV8, AAV9, AAV9 variant, AAV10, AAV11, AAV12, AAV13, AAV-Rh10, AAV-Rh74, AAV-2i8, AAV-DJ, LK03, NP59, or KP1.
Embodiment 14: The isolated nucleic acid molecule of any one of embodiments 9-13, wherein the exogeneous gene has a size from about 2 kilobases to about 7 kilobases.
Embodiment 15: The isolated nucleic acid molecule of embodiment 9, comprising the sequence encoding the AAV rep protein and the AAV capsid.
Embodiment 16: The isolated nucleic acid molecule of embodiment 15, further comprising a sequence encoding a non-AUG translation start codon 5′ of the sequence encoding the AAV rep protein and the sequence encoding the AAV capsid.
Embodiment 17: The isolated nucleic acid molecule of embodiment 16, wherein the non-AUG translation start is ACG.
Embodiment 18: An isolated nucleic acid molecule comprising (a) a sequence encoding an AAV capsid, wherein isolated nucleic acid molecule does not comprise a sequence encoding AAV rep; and (b) an exogenous gene flanked by inverted terminal repeat (ITR) sequences.
Embodiment 19: The isolated nucleic acid molecule of embodiment 18, wherein the AAV capsid comprises a capsid from AAV1, AAV2, AAV2 variant, AAV3A, AAV3B, AAV4, AAV5, AAV6, AAV7, AAV8, AAV9, AAV9 variant, AAV10, AAV11, AAV12, AAV13, AAV-Rh10, AAV-Rh74, AAV-2i8, AAV-DJ, LK03, NP59, or KP1.
Embodiment 20: The isolated nucleic acid molecule of any one of embodiments 18-19, wherein the exogeneous gene has a size from about 2 kilobases to about 7 kilobases.
Embodiment 21: The isolated nucleic acid molecule of any one of embodiments 18-20, further comprising a Ef1α promoter 5′ of the sequence coding the AAV capsid.
Embodiment 22: The isolated nucleic acid molecule of any one of embodiments 18-20, further comprising a p40 promoter 5′ of the sequence coding the AAV capsid.
Embodiment 23: An isolated nucleic acid molecule comprising (a) sequence encoding an AAV rep protein or a sequence encoding an AAV capsid, and a sequence encoding a non-AUG translation start codon 5′ of the sequence encoding an AAV rep protein or a sequence encoding an AAV capsid; and (b) one or more helper elements.
Embodiment 24: The isolated nucleic acid molecule of embodiment 23, comprising the sequence encoding the AAV rep protein.
Embodiment 25: The isolated nucleic acid molecule of embodiment 24, wherein the rep protein comprises rep78, rep68, rep52, and/or rep40.
Embodiment 26: The isolated nucleic acid molecule of embodiment 24, wherein the AAV rep protein comprises REP2.
Embodiment 27: The isolated nucleic acid molecule of embodiment 23, comprising the sequence encoding the AAV capsid.
Embodiment 28: The isolated nucleic acid molecule of embodiment 27, wherein the AAV capsid comprises a capsid from AAV1, AAV2, AAV2 variant, AAV3A, AAV3B, AAV4, AAV5, AAV6, AAV7, AAV8, AAV9, AAV9 variant, AAV10, AAV11, AAV12, AAV13, AAV-Rh10, AAV-Rh74, AAV-2i8, AAV-DJ, LK03, NP59, or KP1.
Embodiment 29: The isolated nucleic acid molecule of embodiment 23, wherein the non-AUG translation start is ACG.
Embodiment 30: An isolated nucleic acid molecule comprising (a) a sequence encoding an AAV rep protein or a sequence encoding an AAV capsid, wherein a promoter is downstream of the sequence encoding the AAV rep protein or sequence encoding the AAV capsid; and (b) one or more helper elements.
Embodiment 31: The isolated nucleic acid molecule of embodiment 30, comprising the sequence encoding the AAV rep protein.
Embodiment 32: The isolated nucleic acid molecule of embodiment 31, wherein the rep protein comprises rep78, rep68, rep52, and/or rep40.
Embodiment 33: The isolated nucleic acid molecule of embodiment 31, wherein the AAV rep protein comprises REP2.
Embodiment 34: The isolated nucleic acid molecule of embodiment 30, comprising the sequence encoding the AAV capsid.
Embodiment 35: The isolated nucleic acid molecule of embodiment 33, wherein the AAV capsid comprises a capsid from AAV1, AAV2, AAV2 variant, AAV3A, AAV3B, AAV4, AAV5, AAV6, AAV7, AAV8, AAV9, AAV9 variant, AAV10, AAV11, AAV12, AAV13, AAV-Rh10, AAV-Rh74, AAV-2i8, AAV-DJ, LK03, NP59, or KP1.
Embodiment 36: The isolated nucleic acid molecule of embodiment 30, wherein the promoter 3′ of the sequence encoding the AAV rep protein or sequence encoding the AAV capsid comprises a p5 promoter.
Embodiment 37: The isolated nucleic acid molecule of any one of embodiments 30-36, wherein the one or more helper elements comprises Ad5-VA, Ad5-E2A(DBP), or Ad5-E4.
Embodiment 38: The isolated nucleic acid molecule of embodiment 30, comprising the sequence encoding the AAV rep protein and the AAV capsid.
Embodiment 39: The isolated nucleic acid molecule of embodiment 38, further comprising a promoter 3′ of the sequence encoding the AAV rep protein and the sequence encoding the AAV capsid.
Embodiment 40: An isolated nucleic acid molecule comprising (a) a sequence encoding an AAV rep protein or a sequence encoding an AAV capsid, wherein a p5 promoter is not 5′ of the sequence encoding the AAV rep protein or sequence encoding the AAV capsid; and (b) one or more helper elements.
Embodiment 41: The isolated nucleic acid molecule of embodiment 40, comprising the sequence encoding the AAV rep protein.
Embodiment 42: The isolated nucleic acid molecule of embodiment 41, wherein the rep protein comprises rep78, rep68, rep52, and/or rep40.
Embodiment 43: The isolated nucleic acid molecule of embodiment 41, wherein the AAV rep protein comprises REP2.
Embodiment 44: The isolated nucleic acid molecule of embodiment 40, comprising the sequence encoding the AAV capsid.
Embodiment 45: The isolated nucleic acid molecule of embodiment 44, wherein the AAV capsid comprises a capsid from AAV1, AAV2, AAV2 variant, AAV3A, AAV3B, AAV4, AAV5, AAV6, AAV7, AAV8, AAV9, AAV9 variant, AAV10, AAV11, AAV12, AAV13, AAV-Rh10, AAV-Rh74, AAV-2i8, AAV-DJ, LK03, NP59, or KP1.
Embodiment 46: The isolated nucleic acid molecule of any one of embodiments 40-45, wherein the one or more helper elements comprises Ad5-VA, Ad5-E2A(DBP), or Ad5-E4.
Embodiment 47: The isolated nucleic acid molecule of embodiment 40, comprising the sequence encoding the AAV rep protein and the AAV capsid.
Embodiment 48: The isolated nucleic acid molecule of embodiment 40, further comprising a promoter 3′ of the sequence encoding the AAV rep protein and the sequence encoding the AAV capsid.
Embodiment 49: An isolated nucleic acid molecule comprising one or more helper elements from Adenovirus 5, wherein (i) the isolated nucleic acid molecule does not comprise an Ad5 Fiber sequence, or (ii) the isolated nucleic acid molecule is less than 16.5 kilobases.
Embodiment 50: The isolated nucleic acid molecule of embodiment 49, wherein the one or more helper elements comprises Ad5-VA, Ad5-E2A(DBP), or Ad5-E4.
Embodiment 51: The isolated nucleic acid molecule of embodiment 50, wherein the isolated nucleic acid molecule does not comprise an Ad5 Fiber sequence.
Embodiment 52: The isolated nucleic acid molecule of embodiment 49-51, wherein the isolated nucleic acid molecule is less than 16, 15.5, 15, 14.5, 14, 13.5, 13, 12.5, 12, 11.5, or 11 kilobases.
Embodiment 53: The isolated nucleic acid molecule of embodiment 49, wherein the isolated nucleic acid molecule does not comprise an Ad5 Fiber sequence, and the isolated nucleic acid molecule is less than 11.3 kilobases.
Embodiment 54: An isolated nucleic acid molecule comprising a sequence encoding an AAV rep protein, wherein the isolated nucleic acid molecule does not comprise a sequence encoding a capsid.
Embodiment 55: The isolated nucleic acid molecule of embodiment 54, wherein the AAV rep protein comprises REP2.
Embodiment 56: The isolated nucleic acid molecule of any one of embodiments 54 or 55, further comprising one or more helper elements.
Embodiment 57: The isolated nucleic molecule of embodiment 56, wherein the one or more helper elements comprises Ad5-VA, Ad5-E2A(DBP), or Ad5-E4.
Embodiment 58: The isolated nucleic molecule of any one of embodiments 54-57, further comprising a p5 promoter 5′ of the sequence encoding the AAV rep protein.
Embodiment 59: The isolated nucleic molecule of any one of embodiments 54-57, further comprising a sequence encoding a non-AUG translation start codon 5′ of the sequence encoding the AAV rep protein.
Embodiment 60: The isolated nucleic molecule of embodiment 59, wherein the non-AUG translation start codon is ACG.
Embodiment 61: A composition comprising (a) a first isolated nucleic acid molecule comprising i) a sequence encoding an AAV rep protein or a sequence encoding an AAV capsid, and ii) an exogenous gene flanked by inverted terminal repeat (ITR) sequences; and (b) a second isolated nucleic acid molecule comprising one or more helper elements, wherein the first nucleic acid molecule and the second nucleic acid molecule are separate nucleic acid molecules.
Embodiment 62: The composition of embodiment 61, wherein the first nucleic acid molecule comprises the sequence encoding the AAV rep protein.
Embodiment 63: The composition of embodiment 62, wherein the AAV rep protein comprises REP2.
Embodiment 64: The composition of embodiment 61, wherein the first nucleic acid molecule comprises the sequence encoding the AAV capsid.
Embodiment 65: The composition of embodiment 64, wherein the first nucleic acid molecule comprises a sequence with at least 70%, 75%, 80%, 85%, 90%, 95%, 96%, 97%, 98%, 99%, or 100% sequence identity of the sequence as set forth in SEQ ID NO: 14.
Embodiment 66: The composition of embodiment 64, wherein the first nucleic acid molecule comprises a sequence with at least 70%, 75%, 80%, 85%, 90%, 95%, 96%, 97%, 98%, 99%, or 100% sequence identity of the sequence as set forth in SEQ ID NO: 18.
Embodiment 67: The composition of embodiment 65 or 66, wherein the second nucleic acid molecule comprises a sequence with at least 70%, 75%, 80%, 85%, 90%, 95%, 96%, 97%, 98%, 99%, or 100% sequence identity of the sequence as set forth in SEQ ID NO: 2.
Embodiment 68: The composition of embodiment 64, wherein the AAV capsid comprises a capsid from AAV1, AAV2, AAV2 variant, AAV3A, AAV3B, AAV4, AAV5, AAV6, AAV7, AAV8, AAV9, AAV9 variant, AAV10, AAV11, AAV12, AAV13, AAV-Rh10, AAV-Rh74, AAV-2i8, AAV-DJ, LK03, NP59, or KP1.
Embodiment 69: The composition of any one of embodiments 61-65, wherein the exogeneous gene has a size from about 2 kilobases to about 7 kilobases.
Embodiment 70: The composition of embodiment 61, wherein the first nucleic acid molecule comprises the sequence encoding the AAV rep protein and the sequence encoding the AAV capsid.
Embodiment 71: The composition of any one of embodiments 61-70, where the one or more helper elements comprises Ad5-VA, Ad5-E2A(DBP), or Ad5-E4.
Embodiment 72: The composition of embodiment 61, wherein the first isolated nucleic acid molecule comprises a sequence with at least 70%, 75%, 80%, 85%, 90%, 95%, 96%, 97%, 98%, 99%, or 100% sequence identity of the sequence as set forth in SEQ ID NO: 4.
Embodiment 73: The composition of embodiment 61, wherein the first isolated nucleic acid molecule comprises a sequence with at least 70%, 75%, 80%, 85%, 90%, 95%, 96%, 97%, 98%, 99%, or 100% sequence identity of the sequence as set forth in SEQ ID NO: 4, 5, 15 or 17.
Embodiment 74: The composition of embodiment 61, wherein the second isolated nucleic acid molecule comprises a sequence with at least 70%, 75%, 80%, 85%, 90%, 95%, 96%, 97%, 98%, 99%, or 100% sequence identity of the sequence as set forth in SEQ ID NO: 7.
Embodiment 75: A composition comprising (a) a first nucleic acid molecule comprising i) a sequence encoding an AAV rep protein or ii) a sequence comprising one or more helper elements; and (b) a second nucleic acid molecule comprising i) sequence encoding an AAV capsid and ii) an exogenous gene flanked by inverted terminal repeat (ITR) sequences, wherein the first nucleic acid and the second nucleic acid are separate nucleic acid molecules.
Embodiment 76: The composition of embodiment 75, wherein the first nucleic acid molecule comprises the sequence encoding the AAV rep protein and the sequence comprising the one or more helper elements.
Embodiment 77: The composition of embodiment 76, wherein the one or more helper elements comprises Ad5-VA, Ad5-E2A(DBP), or Ad5-E4.
Embodiment 78: The composition of embodiment 76 or 77, wherein the AAV rep protein comprises REP2.
Embodiment 79: The composition of any one of embodiments 75-78, wherein the AAV capsid comprises a capsid from AAV1, AAV2, AAV2 variant, AAV3A, AAV3B, AAV4, AAV5, AAV6, AAV7, AAV8, AAV9, AAV9 variant, AAV10, AAV11, AAV12, AAV13, AAV-Rh10, AAV-Rh74, AAV-2i8, AAV-DJ, LK03, NP59, or KP1.
Embodiment 80: The composition of any one of embodiments 75-79, wherein the exogeneous gene has a size from about 2 kilobases to about 7 kilobases.
Embodiment 81: The composition of any one of embodiments 75-80, wherein the first nucleic acid molecule comprises a sequence with at least 70%, 75%, 80%, 85%, 90%, 95%, 96%, 97%, 98%, 99%, or 100% sequence identity of the sequence as set forth in SEQ ID NO: 2 or 3.
Embodiment 82: The composition of any one of embodiments 75-81, wherein the second nucleic acid molecule comprises a sequence with at least 70%, 75%, 80%, 85%, 90%, 95%, 96%, 97%, 98%, 99%, or 100% sequence identity of the sequence as set forth in SEQ ID NO: 4, 5, 15, or 17.
Embodiment 83: The composition of any one of embodiments 75-81, wherein the second nucleic acid molecule comprises a sequence with at least 70%, 75%, 80%, 85%, 90%, 95%, 96%, 97%, 98%, 99%, or 100% sequence identity of the sequence as set forth in SEQ ID NO: 14 or 18.
Embodiment 84: The composition of any one of embodiments 75-82, wherein the first nucleic acid molecule comprises a sequence with at least 70%, 75%, 80%, 85%, 90%, 95%, 96%, 97%, 98%, 99%, or 100% sequence identity of the sequence as set forth in SEQ ID NO: 2, and wherein the second nucleic acid molecule comprises a sequence with at least 70%, 75%, 80%, 85%, 90%, 95%, 96%, 97%, 98%, 99%, or 100% sequence identity of the sequence as set forth in SEQ ID NO: 15.
Embodiment 85: The composition of any one of embodiments 75-82, wherein the first nucleic acid molecule comprises a sequence with at least 70%, 75%, 80%, 85%, 90%, 95%, 96%, 97%, 98%, 99%, or 100% sequence identity of the sequence as set forth in SEQ ID NO: 2, and wherein the second nucleic acid molecule comprises a sequence with at least 70%, 75%, 80%, 85%, 90%, 95%, 96%, 97%, 98%, 99%, or 100% sequence identity of the sequence as set forth in SEQ ID NO: 17.
Embodiment 86: A composition comprising (a) a first nucleic acid molecule comprising i) a sequence encoding an AAV rep protein or a sequence encoding an AAV capsid, wherein a promoter is 3′ of the sequence encoding the AAV rep protein or sequence encoding the AAV capsid; and ii) one or more helper elements; and (b) a second nucleic acid molecule comprising an exogenous gene flanked by inverted terminal repeat (ITR) sequences.
Embodiment 87: The composition embodiment 86, comprising the sequence encoding the AAV rep protein.
Embodiment 88: The composition of embodiment 87, wherein the AAV rep protein comprises REP2.
Embodiment 89: The composition of embodiment 86, comprising the sequence encoding the AAV capsid.
Embodiment 90: The composition of embodiment 89, wherein the AAV capsid comprises a capsid from AAV1, AAV2, AAV2 variant, AAV3A, AAV3B, AAV4, AAV5, AAV6, AAV7, AAV8, AAV9, AAV9 variant, AAV10, AAV11, AAV12, AAV13, AAV-Rh10, AAV-Rh74, AAV-2i8, AAV-DJ, LK03, NP59, or KP1.
Embodiment 91: The composition of embodiment 86, wherein the promoter 3′ of the sequence encoding the AAV rep protein or sequence encoding the AAV capsid comprises a p5 promoter.
Embodiment 92: The composition of any one of embodiments 86-91, wherein the one or more helper elements comprises Ad5-VA, Ad5-E2A(DBP), or Ad5-E4.
Embodiment 93: The composition of embodiment 86, comprising the sequence encoding the AAV rep protein and the AAV capsid.
Embodiment 94: The composition of embodiment 93, further comprising a promoter 3′ of the sequence encoding the AAV rep protein and the sequence encoding the AAV capsid.
Embodiment 95: The composition of any one of embodiments 86-94, wherein the first nucleic acid molecule comprises a sequence with at least 70%, 75%, 80%, 85%, 90%, 95%, 96%, 97%, 98%, 99%, or 100% sequence identity of the sequence as set forth in SEQ ID NO: 1 or 3.
Embodiment 96: The composition of any one of embodiments 86-95, wherein the first nucleic acid molecule comprises a sequence with at least 70%, 75%, 80%, 85%, 90%, 95%, 96%, 97%, 98%, 99%, or 100% sequence identity of the sequence as set forth in SEQ ID NO: 6.
Embodiment 97: A composition comprising (a) a first isolated nucleic acid molecule comprising i) a sequence encoding an AAV rep protein or a sequence encoding an AAV capsid, wherein a p5 promoter is not 5′ of the sequence encoding the AAV rep protein or sequence encoding the AAV capsid and ii) one or more helper elements; and (b) a second nucleic acid molecule comprising an exogenous gene flanked by inverted terminal repeat (ITR) sequences.
Embodiment 98: The composition of embodiment 97, comprising the sequence encoding the AAV rep protein.
Embodiment 99: The composition of embodiment 98, wherein the AAV rep protein comprises REP2.
Embodiment 100: The composition of embodiment 97, comprising the sequence encoding the AAV capsid.
Embodiment 101: The composition of embodiment 100, wherein the AAV capsid comprises a capsid from AAV1, AAV2, AAV2 variant, AAV3A, AAV3B, AAV4, AAV5, AAV6, AAV7, AAV8, AAV9, AAV9 variant, AAV10, AAV11, AAV12, AAV13, AAV-Rh10, AAV-Rh74, AAV-2i8, AAV-DJ, LK03, NP59, or KP1.
Embodiment 102: The composition of any one of embodiments 97-101, wherein the one or more helper elements comprises Ad5-VA, Ad5-E2A(DBP), or Ad5-E4.
Embodiment 103: The composition of embodiment 97, comprising the sequence encoding the AAV rep protein and the AAV capsid.
Embodiment 104: The composition of embodiment 103, further comprising a promoter 3′ of the sequence encoding the AAV rep protein and the sequence encoding the AAV capsid.
Embodiment 105: A cell comprising the isolated nucleic acid molecule of any one of embodiments 1-60.
Embodiment 106: A cell comprising the composition of any one of embodiments 61-104.
Embodiment 107: The cell of embodiment 105 or 106, wherein the cell comprises a HEK293 cell.
Embodiment 108: The cell of embodiment 107, wherein the HEK293 cell comprises a suspension HEK293 cell.
Embodiment 109: A method comprising introducing the isolated nucleic acid molecule of any one of embodiments 1-60 into a cell, thereby generating a cell comprising the isolated nucleic acid molecule of any one of embodiments 1-60.
Embodiment 110: A method comprising introducing the composition of any one of embodiments 61-104 into a cell, thereby generating a cell comprising the composition of any one of embodiments 61-104.
Embodiment 111: The method of embodiment 109 or 110, wherein the introducing comprises transient transfection.
Embodiment 112: The method of any one of embodiments 109-111, wherein the cell comprises a HEK293 cell.
Embodiment 113: The method of embodiment 112, wherein the HEK293 cell comprises a suspension HEK293 cell.
Embodiment 114: A method of producing AAVs, the method comprising using the cell of any one of embodiments 105-108 to generate the AAVs.
Embodiment 115: The method of embodiment 114, wherein the generated AAVs comprise a full/empty ratio of at least 5%, 10%, 20%, 30%, 40%, or 50% prior to any purification steps.
Embodiment 116: The method of embodiment 114 or 115, wherein a production yield of the generated AAVs is at least 1.1-fold, 1.5-fold, 2.0-fold, 2.5-fold, 3.0-fold, 3.5-fold, 4.0-fold, 4.5-fold, or 5.0-fold of a production yield of AAVs generated by a three-plasmid system.
Embodiment 117: A composition of AAVs generated by the method of any one of embodiments 114-116.
Embodiment 118: A method of treating a condition in a subject in need thereof comprising administering the composition of AAVs of embodiment 117 to the subject, thereby treating the condition.
Embodiment 119: Use of the composition of AAVs of embodiment 117 for manufacturing a medicament for treating a condition in a subject in need thereof.
Embodiment 120: A kit comprising (a) a first nucleic acid molecule i) a sequence encoding an AAV rep protein or a sequence encoding an AAV capsid, and ii) an exogenous gene flanked by inverted terminal repeat (ITR) sequences; (b) a second nucleic acid molecule comprising a sequence comprising one or more helper elements, wherein the first nucleic acid molecule and the second nucleic acid molecule are separate nucleic acid molecules; and (c) instructions.
Embodiment 121: The kit of embodiment 120, wherein the first nucleic acid molecule comprises the sequence encoding the AAV rep protein.
Embodiment 122: The kit of embodiment 121, wherein the AAV rep protein comprises REP2.
Embodiment 123: The kit of embodiment 120, wherein the first nucleic acid molecule comprises the sequence encoding the AAV capsid.
Embodiment 124: The kit of embodiment 123, wherein the AAV capsid comprises a capsid from AAV1, AAV2, AAV2 variant, AAV3A, AAV3B, AAV4, AAV5, AAV6, AAV7, AAV8, AAV9, AAV9 variant, AAV10, AAV11, AAV12, AAV13, AAV-Rh10, AAV-Rh74, AAV-2i8, AAV-DJ, LK03, NP59, or KP1.
Embodiment 125: The kit of any one of embodiments 120-124, wherein the exogeneous gene has a size from about 2 kilobases to about 7 kilobases.
Embodiment 126: The kit of embodiment 120, wherein the first nucleic acid molecule comprises the sequence encoding the AAV rep protein and the sequence encoding the AAV capsid.
Embodiment 127: The kit of any one of embodiments 120-126, wherein the one or more helper elements comprises Ad5-VA, Ad5-E2A(DBP), or Ad5-E4.
Embodiment 128: The kit of any one of embodiments 120 and 123-125, wherein the first isolated nucleic acid molecule comprises a sequence with at least 70%, 75%, 80%, 85%, 90%, 95%, 96%, 97%, 98%, 99%, or 100% sequence identity of the sequence as set forth in SEQ ID NO: 4, 5, 15 or 17.
Embodiment 129: The kit of any one of embodiments 120-130, wherein the second isolated nucleic acid molecule comprises a sequence with at least 70%, 75%, 80%, 85%, 90%, 95%, 96%, 97%, 98%, 99%, or 100% sequence identity of the sequence as set forth in SEQ ID NO: 7.
Embodiment 130: A kit comprising (a) a first nucleic acid molecule comprising i) a sequence encoding an AAV rep protein or ii) a sequence comprising one or more helper elements; (b) a second nucleic acid molecule comprising i) a sequence encoding an AAV capsid and ii) an exogenous gene flanked by inverted terminal repeat (ITR) sequences, wherein the first nucleic acid and the second nucleic acid are separate nucleic acid molecules; and (c) instructions.
Embodiment 131: The kit of embodiment 130, wherein the first nucleic acid molecule comprises the sequence encoding the AAV rep protein and the sequence comprising the one or more helper elements.
Embodiment 132: The kit of embodiment 131, wherein the one or more helper elements comprises Ad5-VA, Ad5-E2A(DBP), or Ad5-E4.
Embodiment 133: The kit of embodiment 131 or 132, wherein the AAV rep protein comprises REP2.
Embodiment 134: The kit of any one of embodiments 130-133, wherein the AAV capsid comprises a capsid from AAV1, AAV2, AAV2 variant, AAV3A, AAV3B, AAV4, AAV5, AAV6, AAV7, AAV8, AAV9, AAV9 variant, AAV10, AAV11, AAV12, AAV13, AAV-Rh10, AAV-Rh74, AAV-2i8, AAV-DJ, LK03, NP59, or KP1.
Embodiment 135: The kit of any one of embodiments 130-134, wherein the exogeneous gene has a size from about 2 kilobases to about 7 kilobases.
Embodiment 136: The kit of any one of embodiments 130-135, wherein the first nucleic acid molecule comprises a sequence with at least 70%, 75%, 80%, 85%, 90%, 95%, 96%, 97%, 98%, 99%, or 100% sequence identity of the sequence as set forth in SEQ ID NO: 2 or 3.
Embodiment 137: The kit of any one of embodiments 130-136, wherein the second nucleic acid molecule comprises a sequence with at least 70%, 75%, 80%, 85%, 90%, 95%, 96%, 97%, 98%, 99%, or 100% sequence identity of the sequence as set forth in SEQ ID NO: 4, 5, 15, or 17.
Embodiment 138: The kit of any one of embodiments 130-137, wherein the first nucleic acid molecule comprises a sequence with at least 70%, 75%, 80%, 85%, 90%, 95%, 96%, 97%, 98%, 99%, or 100% sequence identity of the sequence as set forth in SEQ ID NO: 2, and wherein the second nucleic acid molecule comprises a sequence with at least 70%, 75%, 80%, 85%, 90%, 95%, 96%, 97%, 98%, 99%, or 100% sequence identity of the sequence as set forth in SEQ ID NO: 15 or 17.
Embodiment 139: A kit comprising (a) a first nucleic acid molecule comprising i) a sequence encoding an AAV rep protein or a sequence encoding an AAV capsid, wherein a promoter is 3′ of the sequence encoding the AAV rep protein or sequence encoding the AAV capsid; and ii) one or more helper elements; and (b) a second nucleic acid molecule comprising an exogenous gene flanked by inverted terminal repeat (ITR) sequences.
Embodiment 140: The kit embodiment 139, comprising the sequence encoding the AAV rep protein.
Embodiment 141: The kit of embodiment 140, wherein the AAV rep protein comprises REP2.
Embodiment 142: The kit of embodiment 139, comprising the sequence encoding the AAV capsid.
Embodiment 143: The kit of embodiment 142, wherein the AAV capsid comprises a capsid from AAV1, AAV2, AAV2 variant, AAV3A, AAV3B, AAV4, AAV5, AAV6, AAV7, AAV8, AAV9, AAV9 variant, AAV10, AAV11, AAV12, AAV13, AAV-Rh10, AAV-Rh74, AAV-2i8, AAV-DJ, LK03, NP59, or KP1.
Embodiment 144: The kit of embodiment 139, wherein the promoter 3′ of the sequence encoding the AAV rep protein or sequence encoding the AAV capsid comprises a p5 promoter.
Embodiment 145: The kit of any one of embodiments 139-144, wherein the one or more helper elements comprises Ad5-VA, Ad5-E2A(DBP), or Ad5-E4.
Embodiment 146: The kit of embodiment 139, comprising the sequence encoding the AAV rep protein and the AAV capsid.
Embodiment 147: The kit of embodiment 146, further comprising a promoter 3′ of the sequence encoding the AAV rep protein and the sequence encoding the AAV capsid.
Embodiment 148: The kit of any one of embodiments 139-147, wherein the first nucleic acid molecule comprises a sequence with at least 70%, 75%, 80%, 85%, 90%, 95%, 96%, 97%, 98%, 99%, or 100% sequence identity of the sequence as set forth in SEQ ID NO: 1 or 3.
Embodiment 149: The kit of any one of embodiments 139-148, wherein the first nucleic acid molecule comprises a sequence with at least 70%, 75%, 80%, 85%, 90%, 95%, 96%, 97%, 98%, 99%, or 100% sequence identity of the sequence as set forth in SEQ ID NO: 6.
Embodiment 150: A kit comprising: (a) a first isolated nucleic acid molecule comprising i) a sequence encoding an AAV rep protein or a sequence encoding an AAV capsid, wherein a p5 promoter is not 5′ of the sequence encoding the AAV rep protein or sequence encoding the AAV capsid and ii) one or more helper elements; and (b) a second nucleic acid molecule comprising an exogenous gene flanked by inverted terminal repeat (ITR) sequences.
Embodiment 151: The kit of embodiment 150, comprising the sequence encoding the AAV rep protein.
Embodiment 152: The kit of embodiment 151, wherein the AAV rep protein comprises REP2.
Embodiment 153: The kit of embodiment 150, comprising the sequence encoding the AAV capsid.
Embodiment 154: The kit of embodiment 153, wherein the AAV capsid comprises a capsid from AAV1, AAV2, AAV2 variant, AAV3A, AAV3B, AAV4, AAV5, AAV6, AAV7, AAV8, AAV9, AAV9 variant, AAV10, AAV11, AAV12, AAV13, AAV-Rh10, AAV-Rh74, AAV-2i8, AAV-DJ, LK03, NP59, or KP1.
Embodiment 155: The kit of any one of embodiments 150-154, wherein the one or more helper elements comprises Ad5-VA, Ad5-E2A(DBP), or Ad5-E4.
Embodiment 156: The kit of embodiment 150, comprising the sequence encoding the AAV rep protein and the AAV capsid.
Embodiment 157: The kit of embodiment 156, further comprising a promoter 3′ of the sequence encoding the AAV rep protein and the sequence encoding the AAV capsid.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. An isolated nucleic acid molecule comprising
(a) a sequence encoding an AAV capsid, wherein isolated nucleic acid molecule does not comprise a sequence encoding AAV rep; and
(b) an exogenous gene flanked by inverted terminal repeat (ITR) sequences.

2. The isolated nucleic acid molecule of claim 1, wherein the AAV capsid comprises a capsid from AAV1, AAV2, AAV2 variant, AAV3A, AAV3B, AAV4, AAV5, AAV6, AAV7, AAV8, AAV9, AAV9 variant, AAV10, AAV11, AAV12, AAV13, AAV-Rh10, AAV-Rh74, AAV-2i8, AAV-DJ, LK03, NP59, or KP1.

3. The isolated nucleic acid molecule of any one of claims 1-2, wherein the exogeneous gene has a size from about 2 kilobases to about 7 kilobases.

4. The isolated nucleic acid molecule of any one of claims 1-3, further comprising a Ef1α promoter 5′ of the sequence coding the AAV capsid.

5. The isolated nucleic acid molecule of any one of claims 1-4, further comprising a p40 promoter 5′ of the sequence coding the AAV capsid.

6. An isolated nucleic acid molecule comprising a sequence encoding an AAV rep protein, wherein the isolated nucleic acid molecule does not comprise a sequence encoding a capsid.

7. The isolated nucleic acid molecule of claim 6, wherein the AAV rep protein comprises REP2.

8. The isolated nucleic acid molecule of any one of claim 6 or 7, further comprising one or more helper elements.

9. The isolated nucleic molecule of claim 8, wherein the one or more helper elements comprises Ad5-VA, Ad5-E2A(DBP), or Ad5-E4.

10. The isolated nucleic molecule of any one of claims 6-9, further comprising a p5 promoter 5′ of the sequence encoding the AAV rep protein.

11. The isolated nucleic molecule of any one of claims 6-9, further comprising a sequence encoding a non-AUG translation start codon 5′ of the sequence encoding the AAV rep protein.

12. The isolated nucleic molecule of claim 11, wherein the non-AUG translation start codon is ACG.

13. A composition comprising
(a) a first nucleic acid molecule comprising i) a sequence encoding an AAV rep protein or ii) a sequence comprising one or more helper elements; and
(b) a second nucleic acid molecule comprising i) sequence encoding an AAV capsid and ii) an exogenous gene flanked by inverted terminal repeat (ITR) sequences, wherein the first nucleic acid and the second nucleic acid are separate nucleic acid molecules.

14. The composition of claim 13, wherein the first nucleic acid molecule comprises the sequence encoding the AAV rep protein and the sequence comprising the one or more helper elements.

15. The composition of claim 14, wherein the one or more helper elements comprises Ad5-VA, Ad5-E2A(DBP), or Ad5-E4.

16. The composition of any one of claims 13-15, wherein the AAV rep protein comprises REP2.

17. The composition of any one of claims 13-16, wherein the AAV capsid comprises a capsid from AAV1, AAV2, AAV2 variant, AAV3A, AAV3B, AAV4, AAV5, AAV6, AAV7, AAV8, AAV9, AAV9 variant, AAV10, AAV11, AAV12, AAV13, AAV-Rh10, AAV-Rh74, AAV-2i8, AAV-DJ, LK03, NP59, or KP1.

18. The composition of any one of claims 13-17, wherein the exogeneous gene has a size from about 2 kilobases to about 7 kilobases.

19. The composition of any one of claims 13-18, wherein the first nucleic acid molecule comprises a sequence with at least 70%, 75%, 80%, 85%, 90%, 95%, 96%, 97%, 98%, 99%, or 100% sequence identity of the sequence as set forth in SEQ ID NO: 2 or 3.

20. The composition of any one of claims 13-19, wherein the second nucleic acid molecule comprises a sequence with at least 70%, 75%, 80%, 85%, 90%, 95%, 96%, 97%, 98%, 99%, or 100% sequence identity of the sequence as set forth in SEQ ID NO: 4, 5, 15, or 17.

21. The composition of any one of claims 13-19, wherein the second nucleic acid molecule comprises a sequence with at least 70%, 75%, 80%, 85%, 90%, 95%, 96%, 97%, 98%, 99%, or 100% sequence identity of the sequence as set forth in SEQ ID NO: 14 or 18.

22. The composition of any one of claims 13-19, wherein the first nucleic acid molecule comprises a sequence with at least 70%, 75%, 80%, 85%, 90%, 95%, 96%, 97%, 98%, 99%, or 100% sequence identity of the sequence as set forth in SEQ ID NO: 2, and wherein the second nucleic acid molecule comprises a sequence with at least 70%, 75%, 80%, 85%, 90%, 95%, 96%, 97%, 98%, 99%, or 100% sequence identity of the sequence as set forth in SEQ ID NO: 15.

23. The composition of any one of claims 13-22, wherein the first nucleic acid molecule comprises a sequence with at least 70%, 75%, 80%, 85%, 90%, 95%, 96%, 97%, 98%, 99%, or 100% sequence identity of the sequence as set forth in SEQ ID NO: 2, and wherein the second nucleic acid molecule comprises a sequence with at least 70%, 75%, 80%, 85%, 90%, 95%, 96%, 97%, 98%, 99%, or 100% sequence identity of the sequence as set forth in SEQ ID NO: 17.

24. A composition comprising
(a) a first isolated nucleic acid molecule comprising i) a sequence encoding an AAV rep protein or a sequence encoding an AAV capsid, and ii) an exogenous gene flanked by inverted terminal repeat (ITR) sequences; and
(b) a second isolated nucleic acid molecule comprising (A) one or more helper elements, and (B) a sequence encoding an AAV capsid complementing the AAV rep protein in (a) or a sequence encoding an AAV rep protein complementing the AAV capsid in (a);
wherein the first nucleic acid molecule and the second nucleic acid molecule are separate nucleic acid molecules.

25. The composition of claim 24, wherein the first nucleic acid molecule comprises the sequence encoding the AAV rep protein.

26. The composition of claim 25, wherein the AAV rep protein comprises REP2.

27. The composition of claim 24, wherein the first nucleic acid molecule comprises the sequence encoding the AAV capsid.

28. The composition of claim 24, wherein the first nucleic acid molecule comprises a sequence with at least 70%, 75%, 80%, 85%, 90%, 95%, 96%, 97%, 98%, 99%, or 100% sequence identity of the sequence as set forth in SEQ ID NO: 14.

29. The composition of claim 24, wherein the first nucleic acid molecule comprises a sequence with at least 70%, 75%, 80%, 85%, 90%, 95%, 96%, 97%, 98%, 99%, or 100% sequence identity of the sequence as set forth in SEQ ID NO: 18.

30. The composition of claim 25 or 26, wherein the second nucleic acid molecule comprises a sequence with at least 70%, 75%, 80%, 85%, 90%, 95%, 96%, 97%, 98%, 99%, or 100% sequence identity of the sequence as set forth in SEQ ID NO: 2.

31. The composition of claim 24 or 27, wherein the AAV capsid comprises a capsid from AAV1, AAV2, AAV2 variant, AAV3A, AAV3B, AAV4, AAV5, AAV6, AAV7, AAV8, AAV9, AAV9 variant, AAV10, AAV11, AAV12, AAV13, AAV-Rh10, AAV-Rh74, AAV-2i8, AAV-DJ, LK03, NP59, or KP1.

32. The composition of any one of claims 24-31, wherein the exogeneous gene has a size from about 2 kilobases to about 7 kilobases.

33. The composition of claim 24, wherein the first nucleic acid molecule comprises the sequence encoding the AAV rep protein and the sequence encoding the AAV capsid.

34. The composition of any one of claims 24-33, wherein the one or more helper elements comprises Ad5-VA, Ad5-E2A(DBP), or Ad5-E4.

35. The composition of claim 24, wherein the first isolated nucleic acid molecule comprises a sequence with at least 70%, 75%, 80%, 85%, 90%, 95%, 96%, 97%, 98%, 99%, or 100% sequence identity of the sequence as set forth in SEQ ID NO: 4.

36. The composition of claim 24, wherein the first isolated nucleic acid molecule comprises a sequence with at least 70%, 75%, 80%, 85%, 90%, 95%, 96%, 97%, 98%, 99%, or 100% sequence identity of the sequence as set forth in SEQ ID NO: 4, 5, 15 or 17.

37. The composition of claim 24, wherein the second isolated nucleic acid molecule comprises a sequence with at least 70%, 75%, 80%, 85%, 90%, 95%, 96%, 97%, 98%, 99%, or 100% sequence identity of the sequence as set forth in SEQ ID NO: 7.

38. A cell comprising the isolated nucleic acid molecule or the composition of any of the previous claims.

39. The cell of claim 38, wherein the cell comprises a HEK293 cell.

40. The cell of claim 39, wherein the HEK293 cell comprises a suspension HEK293 cell.

41. A method comprising introducing the isolated nucleic acid molecule or the composition of any of the previous claims into a cell, thereby generating a cell comprising the isolated nucleic acid molecule or the composition.

42. The method of claim 41, wherein the introducing comprises transient transfection.

43. The method of any one of claims 41-42, wherein the cell comprises a HEK293 cell.

44. The method of claim 43, wherein the HEK293 cell comprises a suspension HEK293 cell.

45. A method of producing AAVs, the method comprising using the cell of any one of claims 39-40 to generate the AAVs.

46. The method of claim 45, wherein the generated AAVs comprise a full/empty ratio of at least 5%, 10%, 20%, 30%, 40%, or 50% prior to any purification steps.

47. The method of claim 45 or 46, wherein a production yield of the generated AAVs is at least 1.1-fold, 1.5-fold, 2.0-fold, 2.5-fold, 3.0-fold, 3.5-fold, 4.0-fold, 4.5-fold, or 5.0-fold of a production yield of AAVs generated by a three-plasmid system.

48. A composition of AAVs generated by the method of any one of claims 45-47.

49. A method of treating a condition in a subject in need thereof comprising administering the composition of AAVs of claim 48 to the subject, thereby treating the condition.

50. Use of the composition of AAVs of claim 48 in manufacture of a medicament for treating a condition in a subject in need thereof.

51. A kit comprising
the isolated nucleic acid molecule or the composition of any of the previous claims; and instructions.
